(19) United States
(12) Patent Application Publication
(10) Pub. No.: US 2026/0216328 A1
(43) Pub. Date: Jul. 30, 2026

(54) DISRUPTABLE LINKER COMPOSITIONS, SWITCHABLE BISPECIFIC T CELL NANOENGAGER (SWITCH-BITE) COMPOSITIONS COMPRISING THE SAME, AND METHODS OF USE THEREOF

(71) Applicant: The Trustees of the University of Pennsylvania, Philadelphia, PA (US)

(72) Inventors: Michael J. Mitchell, Philadelphia, PA (US); Ningqiang Gong, Philadelphia, PA (US)

(21) Appl. No.: 19/147,696

(22) PCT Filed: Jan. 12, 2024
(86) PCT No.: PCT/US2024/011475

§ 371 Date: Jul. 12, 2025

Related U.S. Application Data
(60) Provisional application No. 63/479,782, filed on Jan. 13, 2023.

(51) Int. Cl. A61K 39/44; A61K 39/00; A61K 47/54; A61K 47/60; A61K 47/61; A61K 47/68; A61P 35/00

(52) U.S. Cl. CPC A61K 39/44; A61K 39/0011; A61K 47/545; A61K 47/60; A61K 47/61; A61K 47/6849; A61K 47/6889; A61P 35/00

(57) ABSTRACT

The present disclosure provides switchable bispecific T cell engagers (switch-BiTEs or SiTEs). In certain embodiments, the switch-BiTEs comprise a disruptable linker. The present disclosure further relates to methods of treating cancer and methods of generating responses in vivo comprising use of the switch-BiTEs of the present disclosure.

### CROSS-REFERENCE TO RELATED APPLICATIONS

This application claims priority under 35 U.S.C. § 119(e) to U.S. Provisional Patent Application No. 63/479,782, filed Jan. 13, 2023, which is incorporated herein by reference in its entirety.

### STATEMENT REGARDING FEDERALLY SPONSORED RESEARCH OR DEVELOPMENT

This invention was made with government support under TR002776 awarded by the National Institutes of Health. The government has certain rights in the invention.

### BACKGROUND

T cell therapy with bispecific T cell engagers (BiTEs) or is an effective treatment for blood malignancies and is currently being developed to treat solid tumors. A key aspect of the antitumor effectiveness of BiTE-based therapy is its ability to engage endogenous T cells and tumor cells by recognizing cell surface antigens on both cells, and then activate the cytotoxic function of the T cells without the need for ex vivo manipulation. Blinatumomab (Blincyto®) is the first BITE to receive FDA approval, and links CD3-positive T cells to CD19-expressing B cell cancers, thereby inducing T-cell activation followed by serial T-cell-mediated lysis of tumor cells. Clinical studies have demonstrated that Blinatumomab can greatly improve B cell acute lymphoblastic leukemia (ALL) treatment. BiTE treatments for glioma, lung cancer, B-cell Lymphoma, prostate cancer, pancreatic cancer, and breast cancers are undergoing current clinical development. Unfortunately, BiTE-based therapies often result in severe adverse effects due to on target, off tumor toxicity, resulting from target antigen expression on both tumor and healthy tissue. This expression pattern is typical for the vast majority of tumor antigens targeted by BiTE and CAR-T therapies. Engineering BiTEs to control the adverse effects of BiTEs is an urgent need in the clinic.

Thus, there is a need in the art for switchable BiTE-based therapies that are able to have their activity modulated in order to reduce off-tumor toxicity and improve their therapeutic potential. The present invention addresses this need.

### BRIEF SUMMARY

In one aspect, the present disclosure provides a disruptable linker comprising a compound of formula (I):

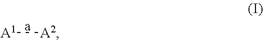

wherein:
A¹ is

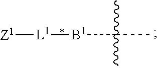

A² is

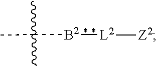

Z¹ and Z² each independently comprise a cross-linking moiety;
L¹ and L² each independently comprise a linker;
B¹ is selected from the group consisting of β-cyclodextrin (β-CD) and cucurbit[8]uril;
B² comprises a β-CD or curcurbit[8]uril binding moiety;
indicates the bond between L¹ and B¹;
* indicates the bond between L² and B²; and
bond a is an optional non-covalent bonding interaction.

In certain embodiments, the compound of formula (I) is a compound of formula (Ia):

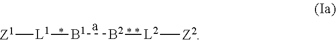

In another aspect, the present disclosure provides a switchable Bispecific T cell Engager (switch-BiTE). In certain embodiments, the switch-BiTE comprises a first binding domain specific for a surface antigen on a target cell. In certain embodiments, the switch-BiTE comprises a second binding domain specific for a surface antigen on an immune effector cell. In certain embodiments, the switch-BiTE comprises the disruptable linker of the present disclosure.

In another aspect, the present disclosure provides a method for generating the switchable bispecific T cell engager (switch-BiTE) of the present disclosure. In certain embodiments, the method comprises conjugating the first and second binding domains with A¹ and A² of the disruptable linker to provide a first engaging fragment and a second engaging fragment, wherein A¹ is conjugated to the first binding domain and A² is conjugated to the second binding domain. In certain embodiments, the method comprises conjugating the first and second binding domains with A¹ and A² of the disruptable linker to provide a first engaging fragment and a second engaging fragment, wherein A¹ is conjugated to the second binding domain and A² is conjugated to the first binding domain. In certain embodiments, the method comprises contacting the first engaging fragment and the second engaging fragment.

In another aspect, the present disclosure provides a method of treating cancer in a subject in need thereof, comprising administering to the subject an effective amount of the switch-BiTE of the present disclosure, thereby treating the cancer.

In another aspect, the present disclosure provides a method for generating an immune response against a target cell in a subject in need thereof. In certain embodiments, the method comprises administering to the subject an amount of the switch-BiTE of the present disclosure sufficient to prime an immune response. In certain embodiments, the method comprises administering to the subject an effective amount of a small molecule capable of disrupting the switch-BiTE.

In another aspect, the present disclosure provides a method for generating an anti-tumor immune response in a subject in need thereof. In certain embodiments, the method comprises administering to the subject an amount of the switch-BiTE of the present disclosure sufficient to prime an anti-tumor immune response. In certain embodiments, the method comprises administering to the subject an effective amount of a small molecule capable of disrupting the switch-BiTE.

### BRIEF DESCRIPTION OF THE FIGURES

The drawings illustrate generally, by way of example, but not by way of limitation, various embodiments of the present application.

FIG. 1: switchable bispecific T cell nanoengager (SiTE) for cancer immunotherapy. SITE was prepared using a supramolecular chemistry-based method to assemble the Fab fragments of antibodies targeting both T cells and tumour cells (1). Once infused into a patient (2), SiTE can then engage both T cells and tumour cells to induce effective tumour cell killing. However, BiTE therapies, including SiTE, may also engage healthy tissue that expresses the target tumour antigen, resulting in on-target, off-tumour toxicity. In addition, intensive tumour cell lysis and over-activation of innate immune cells like monocytes and macrophages can also induce cytokine release syndrome (CRS) and neurotoxicity. However, unlike traditional BiTEs. SiTE can be disassembled using the small molecule amantadine (AMD) (3), halting off-tumour toxicity, CRS and neurotoxicity upon detection (4,5). While infusion of AMD disengages T cells and tumour cells, which may induce tumour relapse, it was found that high doses of SiTE greatly inhibited tumour cell growth and prevented tumour cell re-challenge, as this high dose induced effective in situ vaccination (6-8). PEG, polyethylene glycol; β-CD, β-cyclodextrin; AMD, amantadine; HER2, human epidermal growth factor receptor 2.

FIGS. 2A-2G: synthesis and characterization of SiTE. FIG. 2A: schematic of the synthesis of SiTEs. Phenyl-PEG-MAL conjugated to anti-CD3 Fab and β-CD-PEG-MAL conjugated to anti-HER2 Fab (random rather than site-specific conjugation was used for all experiments described herein) were mixed in PBS and self-assembled into SiTEs via supramolecular interactions between the phenyl group of anti-CD3 Fab-PEG-phenyl and the f-CD of anti-HER2 Fab-PEG-β-CD. The small molecule amantadine (AMD), which has a high affinity for β-CD, can then be used to disturb the interactions between the phenyl group and f-CD to break up the SiTEs. FIG. 2B: representative TEM image of SiTEs (scale bar=100 nm). FIG. 2C: representative DLS histogram of the SiTEs. FIG. 2D: size exclusion chromatography of free CD3 Fab/HER2 Fab mixture and SiTEs. FIG. 2E: AMD-induced disassembly of SiTE was investigated using a two-step separation and quantification process, SiTE or SiTE mixed with increasing amounts of AMD (AMD1, 10 ng; AMD2, 500 ng: AMD3, 1000 ng) confirming the dose-dependent disassembly of SiTE in the presence of AMD. FIG. 2F: disassembly of SiTE by different amounts of AMD was also confirmed using a fluorescence resonance energy transfer experiment. SiTE assembled using Cell tracer 450-modified CD3 Fab and FITC labeled HER2 Fab were mixed with different amounts of AMD and the emission spectra were measured. FIG. 2G: circular dichroism spectra of SiTEs mixed with different amounts of AMD validated dose-dependent disassembly.

FIGS. 3A-3D: conjugation of β-CD to NH₂-PEG-MAL. FIG. 3A: beta cyclodextrins (D-CDs) were first reacted with TsCl and then conjugated to NH₂-PEG-Maleimide. FIGS. 3B-3C: matrix-assisted laser desorption/ionization-time of flight mass spectrometry (MALDI-TOF-MS) data of β-CDs and β-CDs-OTs, confirming a successful reaction. FIG. 3D: MALDI-TOF-MS data of pegylated β-cyclodextrin (β-CD-PEG-MAL) confirming successful conjugation.

FIGS. 4A-4C: preparation of phenyl-PEG-Maleimide. FIG. 4A: 3-phenylpropanoyl chloride was mixed with NH₂-PEG-Maleimide in dichloromethane for 24 h. Free 3-phenylpropanoyl chloride was removed by dialysis. FIGS. 4B-4C: matrix-assisted laser desorption/ionization-time of flight mass spectrometry (MALDI-TOF-MS) data of NH₂-PEG-MAL and phenyl-PEG-MAL, respectively.

FIGS. 5A-5B: preparation and modification of Fab fragments for CD3 and HER-2 antibodies. FIG. 5A: gel electrophoresis confirming successful cutting of CD3 and HER2 antibodies into Fab fragments. FIG. 5B: gel electrophoresis confirming the conjugation of phenyl-PEG-MAL or β-CD-PEG-MAL with Fab fragments for CD3 and HER2, respectively. A complete antibody typically shows a molecular weight of approximately 150 kDa. After excising the Fc region, the resultant F(ab′)2 fragment shows a molecular weight of roughly 100 kDa. Subsequently, following the reduction process, the molecular weight of a single Fab fragment stands at approximately 50 kDa. Modification of phenyl-PEG-MAL or β-CD-PEG-MAL to the Fabs lead to a little increased molecular weight of the Fabs.

FIGS. 6A-6B: characterization of MAL-PEG-Phenyl-modified CD3 and MAL-PEG-CD-modified HER2 Fabs. CD3 Fab and HER2 Fabs were modified with MAL-PEG-Phenyl and MAL-PEG-CD at different Fab to PEG molar ratios (1:1, 1:2, 1:3, and 1:4). FIG. 6A: size exclusion chromatography (SEC) traces of unmodified CD3 Fab, CD3 Fab-PEG-phenyl when Fab was reacted with MAL-PEG-phenyl at ratios of 1:1, 1:2, 1:3, and 1:4. FIG. 6B: SEC traces of unmodified HER2 Fab, HER2 Fab-PEG-CD when Fab was reacted with MAL-PEG-CD at ratios of 1:1, 1:2, 1:3, and 1:4.

FIGS. 7A-7C: characterization of various SiTEs. FIG. 7A: TEM images of SiTE-1, 2, 3, and 4. FIG. 7B: DLS histogram of SiTE-1, 2, 3, and 4. FIG. 7C: quantification of the composition of SiTE-1, 2, 3, and 4 using a two-step separation and quantification method.

FIGS. 8A-8G: AMD-responsivity and stability of the SiTEs. Quantification of the disassembly of various SiTEs using a two-step separation and quantification method. 10 μg of various SiTEs were treated with an excess (500 μg) of AMD, phenylalanine or tyrosine for 2 h and then were loaded on centrifugal filters to collect components with molecular weights below 50 kDa, 50-100 kDa, 100-300 kDa, 300-1000 kDa and above 1000 kDa. The percentages of various components were quantified using BCA assay. FIGS. 8A-8C: average size (measured with DLS) of SiTE-1, 2, and 3 in PBS at 4° C., respectively. FIGS. 8D-8F: stability of SiTE-3 in complete cell culture medium, mouse serum, and human serum, respectively. FIG. 8G: SiTE-3 disassembly kinetics after AMD treatment. A nanoparticle tracking system was used to quantify the number of SiTE-3 particles.

FIGS. 9A-9D: killing assays. E0771-HER2 or E0771 (HER2⁻) cells were incubated with mouse primary T cells and SiTEs for 24 h before flow and cell viability assays were conducted. FIG. 9A: flow dot plot of the E0771-HER2 and T cell mixture after treatment with different SiTEs. FIG. 9B: cell viability of the E0771-HER2 cells after treatment with different SiTEs. FIG. 9C: confocal images of E0771 and T cells in the presence of SiTE-3 (left) and E0771-HER2 and T cell mixture treated with free anti-CD3/HER2 Fab mixture (right). FIG. 9D: E0771-HER2 and T cell mixture pre-treated with PBS or free anti-CD3/HER2 Fab mixture and then treated with 20 ng/mL SiTE-3 for 24 h before cancer cell viability analysis.

FIGS. 10A-10G: characterization of the affinity and avidity of the SiTE. FIG. 10A: the dissociation constants (Kds) of the free CD3 Fab or SiTE against CD3-expression Jurket T cells were determined. FIG. 10B: the dissociation constants (Kds) of HER2 Fab or SiTE against HER2-expressing E0771-HER2 cancer cells were determined. FIGS. 10C-10G: the enhanced binding avidity of SiTE toward CD3 and HER2 antigen is further confirmed using quartz crystal microbalance (QCM). c, binding of CD3 Fab or SiTE to CD3 protein-coated surface. The surface was first rinsed with PBS for 5 min. Then CD3 protein was added to absorb to the surface (15 min), after washing with PBS for 10 min CD3 Fab or SiTE were used, and after about 40 min the surface was washed with PBS to remove non-specific binding. FIG. 10D: binding of HER2 Fab or SiTE to HER2 protein-coated surface. The surface was rinsed with PBS for 5 min. Then HER2 protein was added to absorb to the surface (15 min), after wash with PBS for 10 min, HER2 Fab or SiTE were used, after about 40 min, the surface was washed with PBS to remove non-specific binding. FIGS. 10E-10F show the absorption of free CD3 Fab and free HER2 to the surface, respectively. FIG. 10G shows the binding of free SiTE to the surface.

FIGS. 11A-11F: small molecule AMD controls SiTE activity in vitro. FIG. 11A: schematic showing the mechanism of AMD-controllable SiTE. SiTE can effectively engage T cells and target cells to induce target cell lysis, but this engagement can be broken using the small molecule AMD. FIG. 11B: confocal images of a mixture of E0771-HER2 cancer cells (red) and primary mouse T cells (blue). The cell mixtures were treated with different concentrations of SITE (upper panel) or with 20 ng/mL SiTE mixed with different amounts of AMD (lower panel). FIG. 11C: E0771-HER2-Luc cells were mixed with mouse primary T cells and incubated with a mixture of anti-CD3 Fab and anti-HER2 Fab, different concentrations of SiTE, or a mixture of SiTE and AMD for 24 h, and cancer cell viability was measured. FIG. 11D: IFN-γ levels in the culture medium in c were measured. FIG. 11E: E0771 or E0771-HER2 cells were mixed with mouse primary T cells and treated with 20 ng/mL SiTE in the presence of different concentrations of AMD for 24 h, and tumour cell viability was measured. FIG. 11F: flow dot plots of the E0771-HER2 and T cell mixture after treatment with different formulations (left) and the subsequent quantification (right) (n=3). The data in FIGS. 11C-11F were shown as the mean±s.d. (n=3-5) from independent experiments. Statistical differences were analyzed by two-tailed unpaired Student's t-test.

FIGS. 12A-12K: SiTE induced T cell-T cell engagements but did not elicit a toxic level of cytokine release. FIG. 12A: E0771-HER2 cells (green) and primary T cells (red) were co-cultured and then treated with PBS, SiTE or a mixture of CD3 and HER2 antibody for 2 h. After that, the cells were observed under confocal. T cell to T cell contacts (FIG. 12B) and T cell to tumour cell contacts (c) were quantified manually. In order to investigate the contribution of potential toxicity of SiTE-induced T cell-T cell contacts, primary human CD3 T cells were co-cultured with Raji-Luc-GFP cells and were treated with Blinatumomab, SiTE, or a mixture of CD3 and HER2 antibody. (FIGS. 12D-12F) Cell culture medium was collected and the IL-2 (FIG. 12D). TNF-α (FIG. 12E) and IL-6 (FIG. 12F) levels in the medium were determined using ELISA kits. SiTE or a mixture of CD3 and HER2 antibody was i.v. injected to healthy mice at 0 h. After 24 h, the T cells in the blood were collected and the crosslinking level was determined using flow cytometry (FIG. 12G). The TNF-α (FIG. 12H) and IL-6 (FIG. 12I) levels in the blood, mouse body weight (FIG. 12J) and temperature (FIG. 12K) were also monitored (FIGS. 12H-12I). Data in FIG. 12B and FIG. 12C was shown as mean±SD (n=10), statistic difference was analyzed by two-tailed unpaired Student's t-test. Data in FIGS. 12D-12F are shown as mean±SD (n=4), analyzed by two-tailed unpaired Student's t-test. Data in FIGS. 12H-12I are shown as mean z SD (n=3), analyzed by two-tailed unpaired Student's t-test. P values are indicated.

FIGS. 13A-13D: SiTE engaging T cells and cancer cells and induce toxicity to cancer cells. FIG. 13A: E0771-HER2-Luc cells were incubated with mouse primary T cells and SiTE (20 ng/mL) before cancer cell viability was measured at different timepoints. FIG. 13B: E0771-HER2 cells were incubated with mouse primary T cells and an anti-CD3 Fab and anti-HER2 Fab mixture, different concentrations of SiTE, or a mixture of SiTE and AMD for 24 h. The level of the T cell activation marker, granzyme B, in the culture medium was measured. FIGS. 13C-13D: E0771 or E0771-HER2 cells were mixed with mouse primary T cells and were treated with SiTE (20 ng/mL) or a mixture of SiTE (20 ng/mL) and different concentrations of AMD for 24 h. The granzyme B and IFN-γ levels in the culture medium were measured.

FIGS. 14A-14C: AMD controls the activity of the anti-CD3/CD19 SiTE. FIG. 14A: confocal images of Raji-Luc-GFP cells (a human CD19⁺ cancer cell line) and human primary T cells after treatment with different concentrations of anti-CD3/CD19 SiTE (FIG. 14A, upper panel). AMD-induced separation of cancer cells and T cells was also observed under confocal (FIG. 14A, lower panel). FIG. 14B: cell viability of Raji-Luc-GFP cells after the cell mixtures were treated with indicated groups for 24 h. FIG. 14C: Raji-Luc-GFP cells were mixed with T cells and incubated with 20 ng/mL SiTE or 20 ng/mL SiTE mixed with different concentrations of AMD for 24 h before cell viability was measured.

FIG. 15: hemolytic analysis of mouse red blood cells after incubation with SiTE. Hemolysis percentage was determined by quantifying the release of hemoglobin into the buffer and then plotted as a function of the SiTE concentration. Water and PBS were used as positive and negative controls, respectively.

FIGS. 16A-16B: blood circulation of the SiTE. SiTE was labeled with Cy7 and i.v. injected into mice. 10 μL of blood was collected at the indicated time points, and the concentrations of SiTE in the blood were measured using an IVIS system. FIG. 16A: IVIS image of mouse blood collected at different time points. FIG. 16B: quantification of the percentage of SiTE in circulation at different time points (n=3).

FIGS. 17A-17F: half-life and in vivo biodistribution of Blinatumomab. FIGS. 17A-17B: Blinatumomab was labeled with Cy7 and was i.v. injected to healthy untreated mice. 10 μL of blood at 0, 1, 2, 4, 6, 12, 24, 36 and 48 h was collected and imaged using IVIS. The fluorescence signal at different time points were quantified (FIG. 17B). FIG. 17C: the biodistribution of Blinatumomab in healthy mice was also investigated 24 h after Blinatumomab-Cy7 injection. FIG. 17D: In order to mimic a clinical patient treatment, a mouse model was constructed with CD19 expression in the liver and a subcutaneous B-cell lymphoma model. Blinatumomab-Cy7 was i.v. injected at 0 h. The mice were euthanized at 24 h and the distribution of Blinatumomab-Cy7 in major organs and tumour was evaluated. The Blinatumomab-Cy7 mainly accumulates in the liver, spleen and tumour. The distribution of SiTE that targets human CD3 and human CD19 antigen was evaluated in both healthy untreated mice (FIG. 17E) and in a mouse model with CD19 expression in the liver and has a subcutaneous B-cell lymphoma tumour (FIG. 17F), similar results were obtained.

FIGS. 18A-18F: SiTE localizes in the tumour and spleen. E0771-HER2 tumour-bearing mice were i.v. injected with a Cy7-labeled SiTE (Cy7 labeled on CD3 Fab), a Cy7-labeled Fab nanoparticle made of 50% HER2 Fab isotype and 50% CD3 Fab (Cy7 labeled on CD3 Fab), or a Cy7-labeled Fab nanoparticle made of 50% isotype CD3 Fab and 50% HER2 Fab (Cy7 labeled on CD3 Fab isotype) at 0 h. After 24 h, the mice were euthanized and the Cy7 signal in different organs and tumour was detected. FIG. 18A: IVIS images of major mouse organs and tumour in SiTE-Cy7 group. FIG. 18B: IVIS images of major mouse organs and tumour in Fab nanoparticle made of 50% isotype HER2 Fab and 50% CD3 Fab group. FIG. 18C: IVIS images of major mouse organs and tumour in Fab nanoparticle made of 50% isotype CD3 Fab and 50% HER2 Fab group. FIG. 18D: Tumour-bearing mice were treated with PBS, a protein nanoparticle made of HER2 isotype and CD3 Fab-Cy7, or SiTE-Cy7 at 0 h. After 24 h, mice were euthanized and the Cy7 signal in the tumour tissue was analyzed using immunofluorescence imaging. FIGS. 18E-18F: E0771-HER2 tumour spheroid (cells were labeled with CSFE dye) were treated with Cy5-labelled HER2 antibody (FIG. 18E) or Cy5-labelled SiTE (FIG. 18F) for 2 h and the penetration of these materials in tumour spheroids were investigated using confocal z-stack function.

FIGS. 19A-19J: AMD mediates the in vivo disassembly of SiTE. FIGS. 19A-19C: SiTE was labeled with Cy7 dye (Cy7 was labeled on CD3 Fab) and was i.v. injected to tumour-bearing mice at 0 h. Then. PBS. AMD dispersed in PBS or AMD dispersed in 5% polyoxyethylene castor oil was injected at 18 h post-SiTE-Cy7 injection. 6 h later, mice were euthanized and the Cy7 signal in different organs and tumour was measured using IVIS. FIG. 19D: Half-life of AMD in mouse blood when the AMD is dispersed in PBS or in 5% polyoxyethylene castor oil. When the AMD is dispersed in PBS, the half-life is about 9 hours, which is enhanced to 24 h when it is dispersed in 5% polyoxyethylene castor oil. FIG. 19E: quantification of the fluorescence in tumour tissues in a-c. Data was shown as mean±SD (n=3), statistical differences were analyzed using two-tailed unpaired Student's t-test. FIG. 19F: AMD concentrations in the liver and tumour over time was investigated. FIG. 19G: the distribution of AMD in mice tumour and major organs after 24 h of AMD infusion. In order to investigate the clearance of the disassembled SiTE and the AMD, another animal experiment was performed. SiTE-Cy7 was i.v. infused into tumour bearing mice at 0 h. 4 h later, AMD was infused. After 24 h, mice were euthanized and the Cy7 signal distribution in major organs and mice body was observed. Cy7 signal was detected in the spleen, kidney and bladder. Demonstrate potential clearance from the kidney and urine. A kinetics study was next performed to investigate the Cy7 signal (FIG. 19I) and AMD level in the bladder (FIG. 19J).

FIGS. 20A-20G: AMD controls SiTE activity in vivo. FIG. 20A: 106 E0771-HER2 cells were s.c. injected into the right flank of C57/BL6 mice. When tumour sizes reached 50 mm³ (Day 7), mice were given an i.v. injection of 100 μL of their respective treatments: PBS, SiTE, SiTE+100 μg AMD, SiTE+5 μg AMD, or 100 μg AMD (in 5% polyoxyethylene castor oil). One group received SiTE on Day 7 followed by 100 μg AMD at Day 13. All mice were euthanized at day 23. FIG. 20B: images of mice from all treatment groups on day 23. FIG. 20C: tumour growth curves for different groups. Tumour tissue was isolated on Day 23 and observed for immune cell infiltration. FIG. 20D: repeat of the tumour growth inhibition experiment. FIG. 20E: mouse body weight change during the tumour inhibition experiment. FIG. 20F: percentages of CD45+, CD45+CD3+, CD45+CD3+CD4+/CD8+ cells in the tumour tissues. FIG. 20G: immunofluorescence images of the tumour tissues in different groups with cell nuclei labeled with DAPI (blue) and T cells labeled with anti-CD3 antibody (green). Scale bar: 100 μm. The statistical significance of tumour volume in c and e was analyzed by two-tailed unpaired Student's t-test. **P=0.0013 ****P<0.0001. The statistical significance displayed in f was analyzed by two-tailed unpaired Student's t-test.

FIGS. 21A-21D: quantifications of immune cell populations in tumour tissue after SiTE treatment. FIG. 21A: flow gate strategy for immune cell infiltration analysis. FIGS. 21B-21D: representative flow data showing CD45⁺ cell (FIG. 21B), CD45⁺CD3⁺ cell (FIG. 21C), CD45⁺CD3⁺CD4⁺ cell (FIG. 21D), and CD45⁺CD3⁺CD8⁺ cell infiltration in the tumour tissues with indicated treatments.

FIG. 22: images of H&E-stained sections of E0771-HER2 tumour tissues. H&E-stained sections of E0771-HER2 tumour tissues after the mice were treated with different formulations. Scale bar: 100 μm.

FIGS. 23A-23I: T cell infiltration levels in the tumour tissues before and after SiTE or AMD treatment. The mice were s.c injected with 106 E0771-HER2 cells at day 0, SiTE were injected at day 7, 9, and day 11 and AMD was injected at day 13 FIG. 23A: tumour tissues at day 6, day 12 and day 14 were collected, digested, and filtered, and the immune cell infiltration in the tumour tissue was analyzed using flow (flow gate strategy is shown in FIG. 23B). FIGS. 23C-23E: flow dot plots of CD45+, CD45+CD3+, CD45+CD3+CD4+ or CD45+CD3+CD8+ cells, respectively. FIGS. 23F-23I: quantifications of FIGS. 23C-23E, respectively. The statistical significance of tumour volume in FIGS. 23F-23I was analyzed by two-tailed unpaired Student's t-test. FIG. 23F,***P=0.0007, **P=0.0027. FIG. 23G, ***P=0.0005, *P=0.0324. FIG. 23H, ***P=0.0006, *P=0.0351. FIG. 23I, ***P=0.0005, *P=0.0089.

FIGS. 24A-24J: T cell infiltration in E0771 tumours after SiTE treatment. HER2 non-expression E0771 cells were s.c. injected to mice right flank. The mice were treated with PBS, SiTE or a mixture of SiTE and AMD at day 7, 9 and 11. Mice images at day 17 (FIG. 24A) tumour growth curve (FIG. 24B) and mice body weight (FIG. 24C) were shown. The mice were euthanized at day 17 and T cell infiltrations in the tumour tissue were measured using flow. FIGS. 24D-24H: flow dot plots of CD45⁺, CD45⁺CD3⁺, CD45⁺CD3⁺CD4⁺ or CD45⁺CD3⁺CD8⁺ cells, respectively. FIGS. 24E, 24G, and 24I-24J are quantifications of FIGS. 24D, 24F, and 24H, respectively.

FIGS. 25A-25D: construction of mouse model with liver expression of HER2 antigen using a Piggybac transposon system. FIGS. 25A-25B: structure of the plasmids. The two plasmids pCMV-hyPBase and the pPB7 TBG.human HER2.P2A.IRFP720 were delivered using hydrodynamic injection or using a MC-3 lipid nanoparticle. FIG. 25C: IVIS image of the organs of mice receiving PBS, hydrodynamic injection of the two plasmids, or plasmids encapsulated in a MC-3 lipid nanoparticle. Images were taken three weeks after treatment. FIG. 25D: expression of HER2 antigen in mouse liver in different treatment groups. Scale bar: 100 μm.

FIGS. 26A-26J: AMD reduces the on-target, off-tumour toxicity of SiTE in vivo. FIG. 26A: mouse model with expression of human HER2 antigen in the liver was constructed using a piggyback transposon system delivered via lipid nanoparticles (LNPs) encapsulating two plasmids, pCMV-hyPBase and pPB7 TBG.human HER2.P2A.IRFP720, at day −30. At day 0, 10⁶ E0771-HER2 tumour cells were injected s.c. into the right flank at day 0. After tumour size reached 50 mm³ (day 7), PBS, SiTE, or AMD were i.v. injected three times every two days. When an approximate 15% decrease in body weight indicative of toxicity was observed on day 13, half of the mice that had received SiTE were given an i.v. injection of AMD. FIGS. 26B-26D: mouse tumour growth curves, measurements of mouse body weight, and survival curves, respectively. n=8 mice. P value in FIG. 26D was determined using Log-rank (Mantel-Cox) test. The red stars in FIGS. 26B-26C indicate that mice were euthanized. FIGS. 26E-26K: measurements of markers for inflammation and toxicity including AST, ALT, TNF-α, IFN-γ, and IL-2 levels in mouse blood at different time points. n=3 mice. FIG. 26I: all mice were euthanized by day 19, and mouse livers were harvested. H&E staining was conducted to detect liver damage in different groups. Scale bar: 100 m. FIG. 26M: immunofluorescence imaging of CD3 T cells in liver tissue. Blue: DAPI: Red, CD3⁺ T cells. The data in FIGS. 26B and 26E-26H were shown as the mean±s.d. (n=5). P values were indicated, blue, SiTE vs SiTE+AMD at day 15; pink, Blinatumomab vs SiTE+AMD at day 15, ****P<0.0001, analyzed by two-tailed unpaired Student's t-test.

FIGS. 27A-27C: off-tumour toxicity induced by SiTE can be controlled using AMD. C57BL/6 mice with HER2 expression in the liver were injected with PBS, SiTE, or AMD at day 7, 9 and 11. AMD (in 5% polyoxyethylene castor oil) was administrated at day 13. T cell infiltration in the liver was measured using flow (FIG. 27A). FIG. 27B: liver T cells in different treatment groups at day 13. FIG. 27C: T cell levels in the liver before (day 13) and after (day 15) AMD infusion. P values were indicated in FIGS. 27B-27C, analyzed by two-tailed unpaired Student's t-test. n=3. FIG. 27B, ****P<0.0001, ***P=0.0003; FIG. 27C, ***P=0.0002.

FIG. 28: images of H&E-stained sections of major organs. H&E-stained sections of major organs in E0771-HER2 tumour-bearing mice after treatment with different formulations. Scale bar: 100 μm.

FIGS. 29A-29D: individual E0771-HER2 tumour size curves in each treatment group. CR, complete regression (n=8).

FIGS. 30A-30L: high doses of SiTE induces a strong tumour-specific T cell immune response. FIG. 30A: E0771-HER2 tumor mouse model expressing HER2 in the liver was constructed. 7 days later, mice were treated i.v. injection of PBS, low dose of SiTE (1 mg/kg), high dose of SiTE (5 mg/kg), or AMD at day 7, day 9 and day 11. At Day 13, AMD was given to the groups receiving SiTE. Some mice were given the AMD before day 13 upon the observation of >20% body weight loss. FIG. 30B: tumour growth curves are shown as an average of the 8 mice in each treatment group for days 0-17 and each mouse is graphed separately for days 0-60. In order to assess the mechanism by which the high dose SiTE group showed an improved antitumour effect, an additional animal experiment was performed using the same model and treatment groups, with tumour isolation on Day 17. FIGS. 30C-30F: immune cell infiltration in tumour tissue was analyzed using flow cytometry. CD25⁺FoxP3⁺ regulatory T cell (Treg) numbers and PD-1 expression on T cell surface in each group. FIGS. 30G-30H: the central memory (CD44⁺CD62L⁺) and effector memory (CD44⁺CD62L) cell populations in the mouse spleen. In order to investigate if an immune memory effect was induced. E0771-HER2 tumour-bearing mice were treated with high doses of SiTE and the tumour-free mice in this group were then rechallenged with E0771-HER2 or E0771 cells. Mice pre-treated with either PBS or AMD and challenged with E0771-HER2 were used as controls. Group R1: Healthy mice were treated with PBS at days −43, −41, and −39. The mice were challenged with E0771-HER2 cells at day 0; Group R2: E0771-HER2 tumour-bearing mice were treated with a high dose of SiTE at days −43, −41, and −39, AMD was i.v. injected at day −37. The tumour-free mice were rechallenged with E0771-HER2 cells at day 0; Group R3, E0771-HER2 tumour-bearing mice were pre-treated with high dose of SiTE at days −43, −41, and −39. AMD was i.v. injected at day −37. The mice were rechallenged with E0771-HER2 cells at day 0. Group R4, healthy mice were treated with AMD at day −37 and the mice were challenged with E0771-HER2 cells at day 0. FIGS. 30I-30J: CD8⁺ T cell infiltration in the re-challenged tumour tissue was detected by immunofluorescence. Blue, DAPI; Red, CD8⁺ T cells. FIGS. 30K-30L: CD4⁺ T cell infiltration in the tumour tissue detected by immunofluorescence. Blue. DAPI; Red, CD4⁺ T cells. The data in FIGS. 30D, 30F, 30H, 30J, and 30I were shown as the mean±s.d. (n=3) from three independent experiments. P values were indicated in these figures, analyzed by two-tailed unpaired Student's t-test.

FIGS. 31A-31C: high dose of SiTE elicited antigen-specific immune response to tumours. C57BL/6 mice with liver expression of HER2 were injected with PBS, SiTE low dose, SiTE high dose, or AMD at day 7, 9 and 11. AMD was administrated to SiTE-treated groups once severe toxicity was observed. Mice were euthanized at day 17, and the tumour tissues were collected and analyzed on flow. FIG. 31A: mouse weight during the treatment. FIG. 31B: flow gate strategy. FIG. 31C: CD44+CD62L+ central memory cells in the tumour tissue.

FIGS. 32A-32F: High doses of SiTE enhance DC maturation and antibody production in vivo. FIGS. 32A-32C: E0771-HER2 cells were s.c. injected to mice at day 0. E0771-HER2 tumour-bearing mice were treated with low doses (1 mg/kg) or high doses (5 mg/kg) of SiTE at days 7, 9, and 11. AMD was infused at day 13. Mice were euthanized at day 13 and the dendritic cell maturation levels in the tumour draining lymph nodes were evaluated. PBS or AMD-only were used as two control groups. FIGS. 32D-32F: in order to investigate the humoral immune response induced by SiTE, E0771-HER2 tumour-bearing mice were treated with low doses (1 mg/kg) or high doses (5 mg/kg) of SiTE at days 7, 9, and 11. AMD was infused at day 13. At day 27, the HER2-specific total IgG, IgG2c, and IgG1 levels in mouse blood were determined. P values were indicated in FIGS. 32A-32C, analyzed by two-tailed unpaired Student's t-test. n=3.

FIGS. 33A-33H: tumour cell rechallenging experiment. FIG. 33A: Tumour-free mice from the high dose group were rechallenged with E0771-HER2 or E0771 cells and compared to mice pre-treated with either PBS or AMD challenged with E0771-HER2 as controls. Group R1: Healthy mice were treated with PBS at days −43, −41, and −39. The mice were i.v. injected with E0771-HER2 cells at day 0; Group R2: E0771-HER2 tumour-bearing mice were treated with a high dose of SiTE at days −43, −41, and −39, AMD was i.v. injected at day −37. The tumour-free mice were rechallenged with E0771-HER2 cells at day 0; Group R3, E0771-HER2 tumour-bearing mice were pre-treated with high dose of SiTE at days −43, −41, and −39. AMD was i.v. injected at day −37. The mice were rechallenged with E0771-HER2 cells at day 0. Group R4, healthy mice were treated with AMD at day −37 and the mice were i.v. injected with E0771-HER2 cells at day 0. FIGS. 33B-33E: are individual E0771-HER2 or E0771 tumour size curves in the different treatment groups. CR, complete regression (n=8). The mice were euthanized, and the immune cell infiltration in the tumour tissue was analyzed using flow. FIGS. 33F-33H are Treg percentages CD44⁺CD62L⁺ central memory T cell percentages and CD44⁺CD62L⁻ effector memory T cells in different treatment groups. Data were shown as mean±SD. n=4.

FIGS. 34A-34C: flow gate strategies. FIG. 34A: flow gate strategy used in, flow gate strategy used in FIG. 28. FIG. 34B: flow gate strategy used in FIG. 30E. FIG. 34C: flow gate strategy used in FIG. 30G.

FIGS. 35A-35F: SiTE-induced in situ vaccine effect is dependent on tumour cell lysis. FIG. 35A: E0771 or E0771-HER2 cells were inactivated using formaldehyde and were injected subcutaneously to C57BL/6 mice at day −50. SiTE was administrated at days −43, −41, and −39. At day −37, mice were i.v. injected with 10 mg/kg AMD. Then mice were rechallenged with living E0771 or E0771-HER2 cells. Tumour growth was monitored (FIGS. 35B-35F). Healthy mice treated with PBS at day −50, SiTE at days −43, −41, −39, AMD at day −37, and rechallenged with living E0771 at day 0 was used as a control group.

FIGS. 36A-36C: high doses of SiTE generate tumour-specific T cell immune responses. FIG. 36A: E0771-HER2 tumour-bearing mice were treated with 3 times of high dose SiTE. 7 days-post the last dose, T cells were collected from the mice and were co-cultured with E0771-HER2 cells (express luciferase) for 24 h. E0771-HER2 cell viability was determined. FIGS. 36B-36C: E0771-HER2 target cells (labeled with low level of CSFE) and reference cells (B16 cell line, labeled with high level of CSFE) were i.v. infused into the high dose SiTE-treated mice mentioned above. 24 later, target cell killing was determined using flow cytometry (FIG. 36B). FIG. 36C: quantification of FIG. 36B. Data in FIG. 36A and FIG. 36C are shown as mean±SD, n=4. P values in FIG. 36A and FIG. 36C was determined using two-tailed unpaired student's t-test.

FIGS. 37A-37C: proteomics experiment demonstrate high dose of SiTE treatment induce damage associated molecular pattern (DAMP) and tumour antigen release. E0771-HER2 cells were incubated with mouse T cells in serum-free medium and were treated with a low dose (5 ng/mL) or a high dose (20 ng/mL) of SiTE. After 24 h, the supernatant was collected and the proteins in the medium were analyzed. FIG. 37A: relative abundance of various DAMPs released to cell culture medium in the high dose, low dose SiTE or PBS-treated group. FIGS. 37B-37C: tumour antigen HER2 and tumour neoantigen hmmr and srrml abundance in the cell culture medium in different groups.

FIGS. 38A-38E: SiTE and Blinatumomab induce on-target, off tumour toxicity and cytokine release syndrome in vivo. FIG. 38A: humanized immune system mouse model was constructed by treating the mice with an i.p. injection of Busulfan at day −50 and then an i.v. injection of 10⁵ human CD34⁺ fetal liver cells at day −49. Human CD19 antigen was expressed in the livers of the mice using a piggybac transposon system delivered via lipid nanoparticles (LNPs) encapsulating two plasmids, pCMV-hyPBase pPB CMV-hCD19:T2A:EGFP, at day −35. At day −14, 10⁶ Raji-Luc-GFP tumour cells were i.v. injected. Three doses of Blinatumomab bio-similar antibody (5 mg/kg) or SiTE (5 mg/kg) was i.v. injected to the mice at day 1, day 3, and day 5. AMD was injected to the SiTE treated mice at day 7. FIGS. 38B-38E: mice liver enzymes such as ALT (FIG. 38B) and AST (FIG. 38C) and cytokines such as IL-6 (FIG. 38D) and TNF-α (FIG. 38E) were monitored. Data were shown as the mean±s.d., n=5.

FIGS. 39A-39D: construction of mouse model with liver expression of CD19 antigen using a piggybac transposon system. FIG. 39A: structure of the plasmids. The two plasmids, pCMV-hyPBase and pPB CMV-hCD19:T2A:EGFP, were delivered using a MC3 lipid nanoparticle (2 μg plasmid DNA per mouse). FIG. 39B: IVIS image of various organs of mice receiving PBS or plasmids encapsulated in a MC3 lipid nanoparticle. Images were taken 7 weeks after treatment. FIGS. 39C-39D: IHC images of the expression of HER2 antigen in mouse liver in different treatment groups. Scale bar: 100 μm.

FIGS. 40A-40M: AMD reduces the on-target, off-tumour toxicity of SiTE in vivo. FIG. 40A: a humanized immune system mouse model was constructed by treating the mice with an i.p. injection of Busulfan at day −50 and then an i.v. injection of 10⁵ human CD34⁻ fetal liver cells at day −49. Human CD19 antigen was expressed in the livers of the mice using a piggyback transposon system delivered via lipid nanoparticles (LNPs) encapsulating two plasmids, pCMV-hyPBase and pPB CMV-hCD19:T2A:EGFP, at day −35. At day −14, 10⁶ Raji-Luc-GFP tumour cells were i.v. injected. Before using Blinatumomab or SiTE for cancer treatment, Tocilizumab (10 mg/kg) was administrated to mice to prevent CRS-related symptoms. PBS, Blinatumomab in vivo bio-similar antibody (5 mg/kg), or SiTE (5 mg/kg) were i.v. injected every two days for three total doses. When an approximately 15% decrease in body weight (indicative of toxicity) was observed on day 7, half of the mice that had received SiTE were given an i.v. injection of AMD (in 5% polyoxyethylene castor oil). Throughout the study, mice were euthanized when body weight decreased more than 20%. IVIS was used to monitor tumour burden in vivo. FIG. 40B: shows the IVIS images of the mice at days 0, 7, 10, and 30. FIGS. 40C-40D: mouse body weight and survival curves, respectively. n=10 mice. The red stars in FIGS. 40B-40C: indicate that mice were euthanized. FIGS. 40E-40H: measurements of markers for toxicity and inflammation including AST, ALT. TNF-α, IFN-γ levels in mouse blood at different time points. n=10 mice. FIG. 40I: in order to evaluate liver toxicities induced by various treatments, an additional animal experiment was performed and mouse livers were harvested at day 9. H&E staining was conducted to detect liver damage in different groups. Scale bar: 100 μm. FIG. 40J: immunofluorescence imaging of CD3 T cells in liver tissue. Blue: DAPI: Red, CD3⁺ T cells. FIG. 40K: tumour-free mice from the SiTE+AMD group were rechallenged with 10⁶ Raji-Luc-GFP cells and the tumour burden post Raji-Luc-GFP cell rechallenging was monitored with IVIS. FIGS. 40L-40M: T cells in the tumour-free mice from the SiTE+AMD group were sorted and were co-cultured with Raji-Luc-GFP cells for 24 h and tumour cell viability were determined using a luciferase assay kit (FIG. 40L). T cells from normal humanized mice co-cultured with Raji-Luc-GFP cells were used as a control group. FIG. 40M: IFN-γ concentrations in the cell culture medium were determined using a ELISA kit. The data in FIGS. 40E-40H were plotted as mean±s.d. (n=10) from three independent experiments. P values were indicated in FIGS. 40E-40H analyzed by two-tailed unpaired Student's t-test. Pink, Blinatumomab vs SiTE+AMD at day 9; blue, SiTE vs SiTE+AMD at day 9; ***P=0.0003, ****P<0.0001. P value in FIG. 40D was determined using Log-rank (Mantel-Cox) test, ****P<0.0001. P values in FIG. 40L and FIG. 40M were determined by two-tailed unpaired Student's t-test, ****P<0.0001, ***P=0.0006.

FIGS. 41A-41J: AMD reduces SiTE-induced cytokine release syndrome in vivo. FIG. 41A: NSG-SGM3 mice were treated with Busulfan at day −50 to remove bone marrow. At day −49, 10⁵ human CD34⁺ fetal liver cells were i.v. injected to let the mouse develop a human immune system. 10⁶ Raji-Luc-GFP tumour cells were i.v. injected at day −14. At days 1, 3, and 5, PBS, Blinatumomab (5 mg/kg), or SiTE (5 mg/kg) was i.v. injected. When an approximate 15% decrease in body weight (indicative of toxicity) was observed on day 7, half of the mice that had received SiTE were given an i.v. injection of AMD. Throughout the study, mice were euthanized when body weight decreased more than 20%. An in vivo imaging system (IVIS) was used to monitor tumour burden in vivo. FIG. 41B: shows the IVIS images of the mice at days 0, 7, 10, and 30. FIGS. 41C-41E: Mouse body weight, temperature and survival curves, respectively. n=10 mice. The stars in FIGS. 41B-41C indicate that mice were euthanized. FIGS. 41F-41J: Measurements of markers for CRS including IL-6, IFN-γ, TNF-α, CCL3, and CXCL10 levels in mouse blood at different time points. n=10 mice. The data in FIGS. 41C-41D and FIGS. 41F-41J were plotted as mean±s.d. (n=10) from three independent experiments. P values were indicated in FIG. 41D and FIGS. 41F-41J; blue, SiTE vs SiTE+AMD at day 9; pink, Blinatumomab vs SiTE+AMD at day 9, analyzed by two-tailed unpaired Student's t-test. P value in FIG. 41E was determined using Log-rank (Mantel-Cox) test.

FIGS. 42A-42J: AMD reduces SiTE-induced neurotoxicity in vivo. FIG. 42A: NSG-SGM3 mice were treated with Busulfan at day −50 to remove mouse bone marrow. At day −49, 10⁵ human CD34⁺ fetal liver cells were i.v. injected to let the mouse develop a human immune system. 10⁶ Raji-Luc-GFP tumour cells were i.v. injected at day −14. At days 1, 3, and 5, PBS or SiTE was i.v. injected (in the PBS group, only PBS was injected to tumour-bearing mice). When an approximately 15% decrease in body weight (indicative of toxicity) was observed on day 7, half of the mice that had received SiTE were given an i.v. injection of AMD (10 mg/kg) and half of them were given Tocilizumab (10 mg/kg) for CRS treatment. FIGS. 42B-42E: mouse body weight, temperature, IL-6 levels, and mouse survival curves, respectively. n=10 mice. The red stars in FIG. 42B indicate that mice were euthanized. FIG. 42F: measurements of IL-1 in mouse blood at different time points. n=10 mice. At around day 33 post-SiTE injection, humanized NSG-SGM3 mice that received either PBS or Tocilizumab treatment developed paralysis (FIG. 42G) or experienced a seizure as indicated by movement along the red arrows (FIG. 42H), which are signs of lethal neurological syndrome. However, mice treated with AMD did not develop paralysis and were not observed experiencing seizures indicative of lethal neurological syndrome. Brain H&E staining (FIG. 42I) and human CD68 immunohistochemistry (FIG. 42J) images of mice at day 35. The data in FIGS. 42B-42F were plotted as mean z s.d. (n=10) from three independent experiments. P value in FIG. 42F was determined using Log-rank (Mantel-Cox) test, ****P<0.0001.

FIGS. 43A-43C: comparison of the antitumour efficacy of SITE and Blinatumomab. In order to solely compare the antitumour capacity of Blinatumomab and the SiTE, CD19 antigen was not expressed in mouse liver so BiTNE or SiTE treatment will not induce on-target, off-tumour toxicity. Additionally, normal NSG mice (without humanization) were used to construct the tumour model so the mice will not develop CRS. FIG. 43A: Raji-Luc-GFP cells were i.v. injected to mice at day −14. Tumour bearing mice were treated with Blinatumomab or SiTE at days 1, 3, or 5. FIG. 43B: mice were imaged using IVIS at days 0, 5, 9, and 13 to monitor the tumour burden level. c, Quantification of the luminescence levels in FIG. 43B. The data in FIG. 43C were plotted as mean s.d. (n=5) f. P value in FIG. 43C was determined using one-way ANOVA.

FIGS. 44A-44C: Blinatumomab also induced an in situ vaccine effect. Raji tumour cells and human T cells were co-cultured in serum-free medium and were treated with Blinatumomab for 24 h. After that, the cell culture medium was collected and the proteins in the medium were analyzed using proteomics. FIG. 44A: Large amount of damage associated molecule patterns (DAMPs); and FIG. 44B: tumour associated antigens were detected in the cell culture medium. FIG. 44C: Raji tumour bearing humanized NSG mice were treated with three doses of Blinatumomab. 7 days post the last dose, T cells from the mice were collected and co-cultured with Raji-Luc-GFP cells for 24 h. Then, Raji-Luc-GFP cell viability was determined. T cell isolated from a PBS-treated humanized NSG mice culturing with Raji-Luc-GFP cells were used as a control group. The data in FIG. 44C are presented as mean±s.d. (n=5) f. P value in FIG. 44C was determined using one-way ANOVA.

FIGS. 45A-45C: CRS symptoms cannot be stopped by simply stop the administration of Blinatumomab. FIG. 45A: humanized NSG mice were i.v. injected with Raji-Luc-GFP cells on day −14. Then Blinatumomab were administrated at days 1, 3, or 5. FIG. 45B: IL-6 level in mouse blood were monitored. FIG. 45C: survival curve.

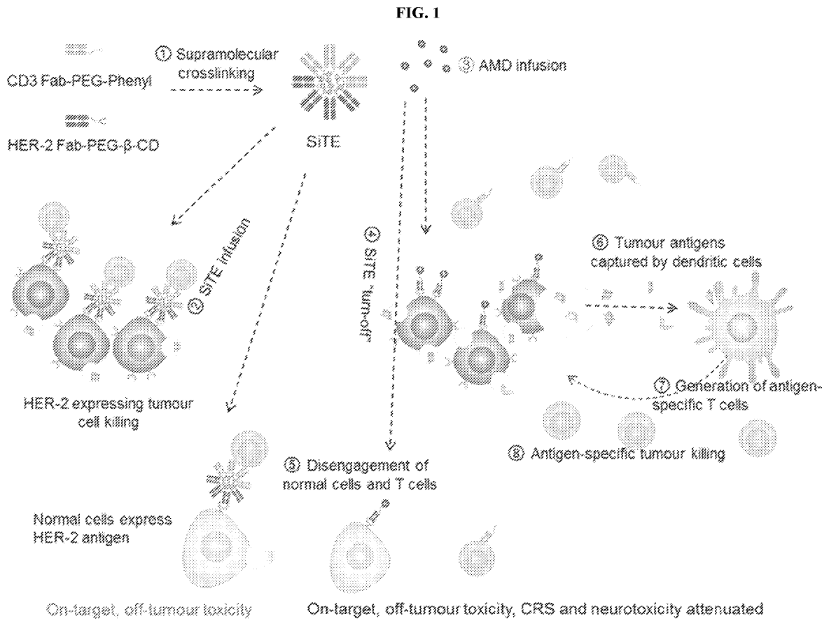

### DETAILED DESCRIPTION OF THE INVENTION

Reference will now be made in detail to certain embodiments of the disclosed subject matter, examples of which are illustrated in part in the accompanying drawings. While the disclosed subject matter will be described in conjunction with the enumerated claims, it will be understood that the exemplified subject matter is not intended to limit the claims to the disclosed subject matter.

Throughout this document, values expressed in a range format should be interpreted in a flexible manner to include not only the numerical values explicitly recited as the limits of the range, but also to include all the individual numerical values or sub-ranges encompassed within that range as if each numerical value and sub-range is explicitly recited. For example, a range of “about 0.1% to about 5%” or “about 0.1% to 5%” should be interpreted to include not just about 0.1% to about 5%, but also the individual values (e.g., 1%, 2%, 3%, and 4%) and the sub-ranges (e.g., 0.1% to 0.5%, 1.1% to 2.2%, 3.3% to 4.4%) within the indicated range. The statement “about X to Y” has the same meaning as “about X to about Y,” unless indicated otherwise. Likewise, the statement “about X, Y, or about Z” has the same meaning as “about X, about Y, or about Z,” unless indicated otherwise.

In this document, the terms “a.” “an.” or “the” are used to include one or more than one unless the context clearly dictates otherwise. The term “or” is used to refer to a nonexclusive “or” unless otherwise indicated. The statement “at least one of A and B” or “at least one of A or B” has the same meaning as “A, B, or A and B.” In addition, it is to be understood that the phraseology or terminology employed herein, and not otherwise defined, is for the purpose of description only and not of limitation. Any use of section headings is intended to aid reading of the document and is not to be interpreted as limiting: information that is relevant to a section heading may occur within or outside of that particular section. All publications, patents, and patent documents referred to in this document are incorporated by reference herein in their entirety, as though individually incorporated by reference.

In the methods described herein, the acts can be carried out in any order, except when a temporal or operational sequence is explicitly recited. Furthermore, specified acts can be carried out concurrently unless explicit claim language recites that they be carried out separately. For example, a claimed act of doing X and a claimed act of doing Y can be conducted simultaneously within a single operation, and the resulting process will fall within the literal scope of the claimed process.

### DESCRIPTION

The present disclosure provides a disruptable linker which in one embodiment comprises a β-cyclodextrin (β-CD) moiety and a β-cyclodextrin binding moiety. Also provided are switchable bispecific antibodies and bispecific T cell nanoengagers (switch-BiTEs) comprising the disruptable linker of the present disclosure. In this way, introduction of a small molecule capable of binding the β-CD moiety with higher affinity than the β-CD binding moiety disrupts the linker and thereby inactivates the activity of the bispecific antibody or switch-BiTE. Also provided are compositions and methods utilizing switch-BiTEs to treat diseases including cancer by inducing T cell mediated toxicity against disease-associated cells including cancer cells. In certain embodiments, the switch-BiTEs of the invention may be suitable for methods of priming endogenous immune responses against target cells.

Cancer immunotherapy with bispecific T cell engagers has shown great promise in the clinic, as it can engage T cells and tumor cells and activate T cells to lyse tumor cells. There are several BiTEs already approved for cancer treatment in the clinic, and a large pipeline of treatments in various stages of clinical trials. However, on target, off tumor toxicity of BiTEs restricts their broader application, as normal tissues can also express tumor-associated antigens that are targeted by BiTEs. In this study, a switchable bispecific T cell nanoengager (switch-BiTE) was prepared for cancer immunotherapy. While the off-tumor toxicity induced by traditional BiTEs is currently difficult to control, the studies disclosed herein demonstrate that the small molecule amantadine (ADM) can be infused to disrupt switch-BiTEs and thus halt off-tumor toxicity upon detection. As such, the switch-BiTEs strategy can greatly improve the therapeutic window of switch-BiTEs. Likewise, the ability to quickly deactivate switch-BiTEs with ADM enables the use of relatively high doses of switch-BiTEs over a short amount of time in order to act as an in situ tumor vaccine via the release of tumor antigens and other danger signals from lysing tumor tissue. The immune responses primed by high-dose switch-BiTE treatment can then act to destroy any remaining tumor cells, and thus prevent relapse. The data disclosed herein also suggests, without wishing to be bound by theory, that these endogenous anti-tumor immune responses are directed against a variety of antigens, which would also lower the likelihood of tumor escape via the mutation or down-regulation of a single tumor antigen. Thus, the studies disclosed herein demonstrate that switch-BiTEs can serve as a broad delivery platform for future bispecific antibody design for cancer immunotherapy, while avoiding associated off-tumor toxicity.

In a solid tumor model, the results disclosed herein demonstrate that a high dose of switch-BiTE+ADM can induce more efficient cancer cell lysis and elicit in situ vaccination effects to effectively inhibit tumor growth, while providing protection from re-challenging. In a humanized immune system mouse model, the results disclosed herein demonstrate the switch-BiTE+ADM strategy induced lower on-target, off-tumor toxicity, CRS, and neurotoxicity when followed by treatment with ADM. Thus, the data of the current disclosure demonstrates that switch-BiTE may serve as a broad delivery platform for future bispecific antibody design for cancer immunotherapy while avoiding the risks of toxicities.

### Definitions

Unless otherwise defined, scientific and technical terms used herein have the meanings that are commonly understood by those of ordinary skill in the art. In the event of any latent ambiguity, definitions provided herein take precedent over any dictionary or extrinsic definition. Unless otherwise required by context, singular terms shall include pluralities and plural terms shall include the singular. The use of “or” means “and/or” unless stated otherwise. The use of the term “including,” as well as other forms, such as “includes” and “included,” is not limiting.

Generally, nomenclature used in connection with cell and tissue culture, molecular biology, immunology, microbiology, genetics and protein and nucleic acid chemistry and hybridization described herein is well-known and commonly used in the art. The methods and techniques provided herein are generally performed according to conventional methods well known in the art and as described in various general and more specific references that are cited and discussed throughout the present specification unless otherwise indicated. Enzymatic reactions and purification techniques are performed according to manufacturer's specifications, as commonly accomplished in the art or as described herein. The nomenclatures used in connection with, and the laboratory procedures and techniques of, analytical chemistry, synthetic organic chemistry, and medicinal and pharmaceutical chemistry described herein are those well-known and commonly used in the art. Standard techniques are used for chemical syntheses, chemical analyses, pharmaceutical preparation, formulation, and delivery, and treatment of patients.

The term “about” as used herein can allow for a degree of variability in a value or range, for example, within 10%, within 5%, or within 1% of a stated value or of a stated limit of a range, and includes the exact stated value or range.

“Activation,” as used herein, refers to the state of a T cell that has been sufficiently stimulated to induce detectable cellular proliferation. Activation can also be associated with induced cytokine production, and detectable effector functions. The term “activated T cells” refers to, among other things, T cells that are undergoing cell division.

As used herein, the term “alkenyl,” employed alone or in combination with other terms, means, unless otherwise stated, a stable monounsaturated or diunsaturated straight chain or branched chain hydrocarbon group having the stated number of carbon atoms.

Examples include vinyl, propenyl (or allyl), crotyl, isopentenyl, butadienyl, 1,3-pentadienyl, 1,4-pentadienyl, and the higher homologs and isomers. A functional group representing an alkene is exemplified by —CH₂—CH═CH₂.

As used herein, the term “alkenylene”, employed alone or in combination with other terms, means, unless otherwise stated, a stable mono-unsaturated or di-unsaturated straight chain or branched chain hydrocarbon group having the stated number of carbon atoms wherein the group has two open valencies.

As used herein, the term “alkoxy” employed alone or in combination with other terms means, unless otherwise stated, an alkyl group having the designated number of carbon atoms, as defined elsewhere herein, connected to the rest of the molecule via an oxygen atom, such as, for example, methoxy, ethoxy, 1-propoxy, 2-propoxy (or isopropoxy) and the higher homologs and isomers. A specific example is (C₁-C₃)alkoxy, such as, but not limited to, ethoxy and methoxy.

As used herein, the term “alkyl” by itself or as part of another substituent means, unless otherwise stated, a straight or branched chain hydrocarbon having the number of carbon atoms designated (i.e., C₁-C₁₀ means one to ten carbon atoms) and includes straight, branched chain, or cyclic substituent groups. Examples include methyl, ethyl, propyl, isopropyl, butyl, isobutyl, tert-butyl, pentyl, neopentyl, hexyl, and cyclopropylmethyl. A specific embodiment is (C₁-C₆) alkyl, such as, but not limited to, ethyl, methyl, isopropyl, isobutyl, n-pentyl, n-hexyl and cyclopropylmethyl.

As used herein, the term “alkylene” by itself or as part of another substituent means, unless otherwise stated, a straight or branched hydrocarbon group having the number of carbon atoms designated (i.e., C₁-C₁₀ means one to ten carbon atoms) and includes straight, branched chain, or cyclic substituent groups, wherein the group has two open valencies. Examples include methylene, 1,2-ethylene, 1,1-ethylene, 1,1-propylene, 1,2-propylene and 1,3-propylene.

As used herein, the term “alkynyl” employed alone or in combination with other terms means, unless otherwise stated, a stable straight chain or branched chain hydrocarbon group with a triple carbon-carbon bond, having the stated number of carbon atoms. Non-limiting examples include ethynyl and propynyl, and the higher homologs and isomers. The term “propargylic” refers to a group exemplified by —CH₂—C≡CH. The term “homopropargylic” refers to a group exemplified by —CH₂CH₂—C≡CH.

As used herein, the term “alkynylene”, employed alone or in combination with other terms, means, unless otherwise stated, a stable straight chain or branched chain hydrocarbon group with a triple carbon-carbon bond, having the stated number of carbon atoms wherein the group has two open valencies.

As used herein, to “alleviate” a disease means reducing the severity of one or more symptoms of the disease.

The term “antibody,” as used herein, refers to an immunoglobulin molecule which specifically binds with an antigen. Antibodies can be intact immunoglobulins derived from natural sources or from recombinant sources and can be immunoreactive portions of intact immunoglobulins. Antibodies are typically tetramers of immunoglobulin molecules comprising two heavy chain and two light chain polypeptides. Each polypeptide chain contains three complementarity-determining regions (CDRs), which bind to the antigen and defines the antibody's antigen specificity.

As used herein, the term “antibody” and “antibodies” can also include polypeptides or polypeptide complexes derived from full-length antibodies. These polypeptide complexes may be naturally occurring or constructed from single chain antibodies or antibody fragments and retain an antigen-specific binding ability. The antibodies of the present invention may exist in a variety of forms including, for example, polyclonal antibodies, monoclonal antibodies, Fv, Fab and F(ab′)2, as well as single chain antibodies (scFv), caninized antibodies, canine antibodies, humanized antibodies, and human antibodies (Harlow et al., 1999, In: Using Antibodies: A Laboratory Manual, Cold Spring Harbor Laboratory Press, NY; Harlow et al., 1989. In: Antibodies: A Laboratory Manual, Cold Spring Harbor, New York: Houston et al., 1988, Proc. Natl. Acad. Sci. USA 85:5879-5883; Bird et al., 1988, Science 242:423-426).

The term “antibody fragment” refers to a polypeptide comprising or derived from a portion of an intact antibody and comprises the antigen-binding determining variable regions of an intact antibody. Examples of antibody fragments include, but are not limited to, Fab, Fab′, F(ab′)₂, and Fv fragments, linear antibodies, scFv antibodies, single-domain antibodies, such as camelid antibodies (Riechmann, 1999, Journal of Immunological Methods 231:25-38), composed of either a VL or a VH domain which exhibit sufficient affinity for the target, and multispecific antibodies formed from antibody fragments. The antibody fragment also includes a human antibody or a humanized antibody or a portion of a human antibody or a humanized antibody.

An “antibody heavy chain,” as used herein, refers to the larger of the two types of polypeptide chains present in all antibody molecules in their naturally occurring conformations.

An “antibody light chain,” as used herein, refers to the smaller of the two types of polypeptide chains present in all antibody molecules in their naturally occurring conformations. K and X light chains refer to the two major antibody light chain isotypes.

By the term “synthetic antibody” as used herein, is meant an antibody which is generated using recombinant DNA technology, such as, for example, an antibody expressed by a bacteriophage as described herein. The term should also be construed to mean an antibody which has been generated by the synthesis of a DNA molecule encoding the antibody and which DNA molecule expresses an antibody protein, or an amino acid sequence specifying the antibody, wherein the DNA or amino acid sequence has been obtained using synthetic DNA or amino acid sequence technology which is available and well known in the art.

The term “antigen” as used herein is defined as a molecule that provokes an immune response. This immune response may involve either antibody production, or the activation of specific immunologically-competent cells, or both. The skilled artisan will understand that any macromolecule, including virtually all proteins or peptides, can serve as an antigen.

Furthermore, antigens can be derived from recombinant or genomic DNA. A skilled artisan will understand that any DNA, which comprises a nucleotide sequences or a partial nucleotide sequence encoding a protein that elicits an immune response therefore encodes an “antigen” as that term is used herein. Furthermore, one skilled in the art will understand that an antigen need not be encoded solely by a full length nucleotide sequence of a gene. It is readily apparent that the present invention includes, but is not limited to, the use of partial nucleotide sequences of more than one gene and that these nucleotide sequences are arranged in various combinations to elicit the desired immune response. Moreover, a skilled artisan will understand that an antigen need not be encoded by a “gene” at all. It is readily apparent that an antigen can be generated synthesized or can be derived from a biological sample. Such a biological sample can include, but is not limited to a tissue sample, a tumor sample, a cell or a biological fluid.

The term “anti-tumor effect” as used herein, refers to a biological effect which can be manifested by a decrease in tumor volume, a decrease in the number of tumor cells, a decrease in the number of metastases, an increase in life expectancy, or amelioration of various physiological symptoms associated with the cancerous condition. An “anti-tumor effect” can also be manifested by the ability of the peptides, polynucleotides, cells and antibodies of the invention in prevention of the occurrence of tumor in the first place.

The term “therapeutic effect” as used herein refers to a consequence of treatment, the results of which are judged to be desirable and beneficial. A therapeutic effect may include, directly or indirectly, the arrest, reduction, or elimination of a disease manifestation. A therapeutic effect may also include, directly or indirectly, the arrest reduction or elimination of the progression of a disease manifestation.

As used herein, the term “aromatic” refers to a carbocycle or heterocycle with one or more polyunsaturated rings and having aromatic character, i.e., having (4n+2) delocalized π (pi) electrons, where ‘n’ is an integer.

As used herein, the term “aryl” employed alone or in combination with other terms means, unless otherwise stated, a carbocyclic aromatic system containing one or more rings (typically one, two or three rings) wherein such rings may be attached together in a pendent manner, such as a biphenyl, or may be fused, such as naphthalene. Examples include phenyl, anthracyl and naphthyl. Aryl groups also include, for example, phenyl or naphthyl rings fused with one or more saturated or partially saturated carbon rings (e.g., bicyclo[4.2.0]octa-1,3,5-trienyl, or indanyl), which can be substituted at one or more carbon atoms of the aromatic and/or saturated or partially saturated rings.

As used herein, the term “aryl-(C₁-C₆)alkyl” or “aralkyl” refers to a functional group wherein a one to six carbon alkylene chain is attached to an aryl group, e.g., —CH₂CH₂-phenyl or —CH₂-phenyl (or benzyl). Specific examples are aryl-CH₂— and aryl-CH(CH₃)—. The term “substituted aryl-(C₁-C₆)alkyl” refers to an aryl-(C₁-C₆)alkyl functional group in which the aryl group is substituted. A specific example is substituted aryl(CH₂)—. Similarly, the term “heteroaryl-(C₁-C₆)alkyl” refers to a functional group wherein a one to three carbon alkylene chain is attached to a heteroaryl group, e.g., —CH₂CH₂-pyridyl. A specific example is heteroaryl-(CH₂)—. The term “substituted heteroaryl-(C₁-C₆)alkyl” refers to a heteroaryl-(C₁-C₆)alkyl functional group in which the heteroaryl group is substituted. A specific example is substituted heteroaryl-(CH₂)—.

As used herein, the term “autologous” is meant to refer to any material derived from the same individual to which it is later to be re-introduced into the individual.

The term “β-cyclodextrin binding moiety” or “β-CD binding moiety” refers to a chemical species, or fragment thereof, which binds to at least a portion of β-CD binding moiety. In certain embodiments, the D-CD binding moiety has a high affinity for β-CD.

A “bispecific antibody,” as used herein, refers to an antibody having binding specificities for at least two different antigenic epitopes. In one embodiment, the epitopes are from the same antigen. In another embodiment, the epitopes are from two different antigens. Methods for making bispecific antibodies are known in the art. For example, bispecific antibodies can be produced recombinantly using the co-expression of two immunoglobulin heavy chain/light chain pairs. See, e.g., Milstein et al. (1983) Nature 305: 537-39. Alternatively, bispecific antibodies can be prepared using chemical linkage. See. e.g., Brennan et al. (1985) Science 229:81. Bispecific antibodies include bispecific antibody fragments. See, e.g., Holliger et al. (1993) Proc. Natl. Acad. Sci. U.S.A. 90:6444-48, Gruber et al. (1994) J. Immunol. 152:5368.

The terms “bispecific T cell engager” or BiTE or “bispecific T cell nanoengager” or switch-BiTE, as used interchangeably herein, refers to a bispecific antigen binding molecule comprising at least two antigen-binding domains, in which one domain binds specifically to a T cell epitope and the other domain binds specifically to an epitope on a target cell. The binding of both the T cell epitope and the target cell epitope simultaneously has the effect of activating the function of the T cell against the target cell. Typically, the T cell is a CD8⁺ T cell, and activation by the BiTE induces cytotoxic function against the target cell. Thus BiTEs or switch-BiTEs are able to cause target-specific T cell cytotoxicity without the need for priming a T cell response by antigen-presenting cells or the recognition of MHC/HLA complexes on the target cell by the TCR of the T cell. Target-cell binding domains may take the form of antibodies, antibody fragments such as Fabs, single-chain antibodies, or single-domain antibodies and the like. Target-cell binding domains may also take the form of ligands for receptors on the surface of the target cells.

The term “cancer” as used herein is defined as disease characterized by the rapid and uncontrolled growth of aberrant cells. Cancer cells can spread locally or through the bloodstream and lymphatic system to other parts of the body. Examples of various cancers include but are not limited to, breast cancer, prostate cancer, ovarian cancer, cervical cancer, skin cancer, pancreatic cancer, colorectal cancer, renal cancer, liver cancer, brain cancer, lymphoma, leukemia, lung cancer and the like. In certain embodiments, the cancer is medullary thyroid carcinoma.

By the term “synthetic antibody” as used herein, is meant an antibody which is generated using recombinant DNA technology, such as, for example, an antibody expressed by a bacteriophage as described herein. The term should also be construed to mean an antibody which has been generated by the synthesis of a DNA molecule encoding the antibody and which DNA molecule expresses an antibody protein, or an amino acid sequence specifying the antibody, wherein the DNA or amino acid sequence has been obtained using synthetic DNA or amino acid sequence technology which is available and well known in the art.

“Co-stimulatory ligand”, as the term is used herein, includes a molecule on an antigen presenting cell (e.g., an aAPC, dendritic cell, B cell, and the like) that specifically binds a cognate co-stimulatory molecule on a T cell, thereby providing a signal which, in addition to the primary signal provided by, for instance, binding of a TCR/CD3 complex with an MHC molecule loaded with peptide, mediates a T cell response, including, but not limited to, proliferation, activation, differentiation, and the like. A co-stimulatory ligand can include, but is not limited to, CD7, B7-1 (CD80), B7-2 (CD86), PD-L1, PD-L2, 4-1BBL. OX40L, inducible costimulatory ligand (ICOS-L), intercellular adhesion molecule (ICAM), CD30L, CD40, CD70, CD83, HLA-G, MICA, MICB, HVEM, lymphotoxin beta receptor, 3/TR6, ILT3, ILT4, HVEM, an agonist or antibody that binds Toll ligand receptor and a ligand that specifically binds with B7-H3. A co-stimulatory ligand also encompasses, inter alia, an antibody that specifically binds with a co-stimulatory molecule present on a T cell, such as, but not limited to, CD27, CD28, 4-1BB, OX40, CD30, CD40L, PD-1, ICOS, lymphocyte function-associated antigen-1 (LFA-1), CD2, CD7, LIGHT, NKG2C, B7-H3, and a ligand that specifically binds with CD83.

A “co-stimulatory molecule” refers to cell-surface molecules expressed by T cells that specifically bind with co-stimulatory ligands expressed by antigen-presenting cells (APCs), thereby providing a “secondary signal” which, in combination with the “primary signal” delivered through MHC/HLA-antigen interactions with the T Cell Receptor (TCR) results in optimal T cell activation including, but not limited to, cytokine production and proliferation. Co-stimulatory molecules include, but are not limited to CD27, CD28, 4-1BB, OX40, CD30, CD40L, PD-1, ICOS, lymphocyte function-associated antigen-1 (LFA-1), CD2, CD7, LIGHT, NKG2C, B7-H3, and a ligand that specifically binds with CD83.

The term “cross-linking moiety” as used herein refers to a fragment of a molecule (i.e. functional group) which enables formation of a covalent bond with a molecule and/or fragment thereof comprising a complementary moiety, upon contact of the cross-linking moiety with said complementary moiety via any one of a number of covalent bond forming reactions (e.g., 1,4-conjugate addition. [4+2] cycloaddition. [3+2]-cycloaddition, and condensation, inter alia). For example, a cross-linking moiety may comprise a maleimide, wherein the complementary moiety is a thiol, and the covalent bond forming reaction is a 1,4-conjugate addition (i.e., Michael addition). Alternatively, a cross-linking moiety may comprise a thiol, wherein the complementary moiety is a maleimide, and the covalent bond forming reaction is a 1,4-conjugate addition. Additional examples of cross-linking moieties, complementary moieties, and corresponding covalent bond forming reactions include, but are not limited to, an alkene or alkyne, an azide, and a [3+2] cycloaddition (i.e., “click” reaction), and an alkene or alkyne, a diene, and a [4+2] cycloaddition (i.e., Diels-Alder cycloaddition).

As used herein, the term “cycloalkyl” by itself or as part of another substituent refers to, unless otherwise stated, a cyclic chain hydrocarbon having the number of carbon atoms designated (i.e., C₃-C₆ refers to a cyclic group comprising a ring group consisting of three to six carbon atoms) and includes straight, branched chain or cyclic substituent groups. Examples of (C₃-C₆)cycloalkyl groups are cyclopropyl, cyclobutyl, cyclopentyl and cyclohexyl. Cycloalkyl rings can be optionally substituted. Non-limiting examples of cycloalkyl groups include: cyclopropyl, 2-methyl-cyclopropyl, cyclopropenyl, cyclobutyl, 2,3-dihydroxycyclobutyl, cyclobutenyl, cyclopentyl, cyclopentenyl, cyclopentadienyl, cyclohexyl, cyclohexenyl, cycloheptyl, cyclooctanyl, decalinyl. 2,5-dimethylcyclopentyl, 3,5-dichlorocyclohexyl, 4-hydroxycyclohexyl, 3,3,5-trimethylcyclohex-1-yl, octahydropentalenyl, octahydro-1H-indenyl, 3a,4,5,6,7,7a-hexahydro-3H-inden-4-yl, decahydroazulenyl; bicyclo[6.2.0]decanyl, decahydronaphthalenyl, and dodecahydro-1H-fluorenyl. The term “cycloalkyl” also includes bicyclic hydrocarbon rings, non-limiting examples of which include, bicyclo-[2.1.1]hexanyl, bicyclo[2.2.1]heptanyl, bicyclo[3.1.1]heptanyl, 1,3-dimethyl[2.2.1]heptan-2-yl, bicyclo[2.2.2]octanyl, and bicyclo[3.3.3]undecanyl.

A “disease” is a state of health of an animal wherein the animal cannot maintain homeostasis, and wherein if the disease is not ameliorated then the animal's health continues to deteriorate. In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

The term “disrupting agent” or “β-CD disrupting agent” as used herein refers to a chemical species which has a β-CD binding affinity sufficient to displace and/or competitively inhibit binding of an alternative chemical species (e.g., a β-CD binding moiety). In certain embodiments, the disrupting agent is amantadine (i.e., adamantan-1-amine).

The term “dysregulated” when used in the context of the level of expression or activity of a gene or protein refers to the level of expression or activity that is different from the expression level or activity of that gene or protein in an otherwise identical healthy animal, organism, tissue, cell or component thereof. The term “dysregulated” also refers to the altered regulation of the level of expression and activity of a gene or protein, compared to the regulation in an otherwise identical healthy animal, organism, tissue, cell or component thereof. Dysregulation of otherwise normal genes and proteins often occurs in cancer cells.

The term “downregulation” as used herein refers to the decrease or elimination of gene expression of one or more genes.

“Effective amount” or “therapeutically effective amount” are used interchangeably herein, and refer to an amount of a compound, formulation, material, or composition, as described herein effective to achieve a particular biological result or provides a therapeutic or prophylactic benefit. Such results may include, but are not limited to an amount that when administered to a mammal, causes a detectable level of immune suppression or tolerance compared to the immune response detected in the absence of the composition of the invention. The immune response can be readily assessed by a plethora of art-recognized methods. The skilled artisan would understand that the amount of the composition administered herein varies and can be readily determined based on a number of factors such as the disease or condition being treated, the age and health and physical condition of the mammal being treated, the severity of the disease, the particular compound being administered, and the like.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

As used herein “endogenous” refers to any material from or produced inside an organism, cell, tissue or system.

The term “epitope” as used herein is defined as a small chemical molecule on an antigen that can elicit an immune response, inducing B and/or T cell responses. An antigen can have one or more epitopes. Most antigens have many epitopes: i.e., they are multivalent. In general, an epitope is roughly about 10 amino acids and/or sugars in size. Preferably, the epitope is about 4-18 amino acids, more preferably about 5-16 amino acids, and even more most preferably 6-14 amino acids, more preferably about 7-12, and most preferably about 8-10 amino acids. One skilled in the art understands that generally the overall three-dimensional structure, rather than the specific linear sequence of the molecule, is the main criterion of antigenic specificity and therefore distinguishes one epitope from another. Based on the present disclosure, a peptide used in the present invention can be an epitope.

As used herein, the term “exogenous” refers to any material introduced from or produced outside an organism, cell, tissue or system.

The term “expand” as used herein refers to increasing in number, as in an increase in the number of T cells. In one embodiment, the T cells that are expanded ex vivo increase in number relative to the number originally present in the culture. In another embodiment, the T cells that are expanded ex vivo increase in number relative to other cell types in the culture. The term “ex vivo,” as used herein, refers to cells that have been removed from a living organism, (e.g., a human) and propagated outside the organism (e.g., in a culture dish, test tube, or bioreactor).

The term “expression” as used herein is defined as the transcription and/or translation of a particular nucleotide sequence driven by its promoter.

“Expression vector” refers to a vector comprising a recombinant polynucleotide comprising expression control sequences operatively linked to a nucleotide sequence to be expressed. An expression vector comprises sufficient cis-acting elements for expression: other elements for expression can be supplied by the host cell or in an in vitro expression system. Expression vectors include all those known in the art, such as cosmids, plasmids (e.g., naked or contained in liposomes) and viruses (e.g., Sendai viruses, lentiviruses, retroviruses, adenoviruses, and adeno-associated viruses) that incorporate the recombinant polynucleotide.

As used herein, the term “halide” refers to a halogen atom bearing a negative charge. The halide anions are fluoride (F⁻), chloride (Cl⁻), bromide (Br⁻), and iodide (I⁻).

As used herein, the term “halo” or “halogen” alone or as part of another substituent refers to, unless otherwise stated, a fluorine, chlorine, bromine, or iodine atom.

The term “haloalkyl” as used herein, includes mono-halo alkyl groups, poly-halo alkyl groups wherein all halo atoms can be the same or different, and per-halo alkyl groups, wherein all hydrogen atoms are replaced by independently selected and/or the same halogen atoms, such as fluoro. Examples of haloalkyl include trifluoromethyl, 1,1-dichloroethyl. 1,2-dichloroethyl, 1,3-dibromo-3,3-difluoropropyl, perfluorobutyl, and the like.

As used herein, the term “heteroalkyl” by itself or in combination with another term means, unless otherwise stated, a stable straight or branched chain alkyl group consisting of the stated number of carbon atoms and one or two heteroatoms selected from the group consisting of O, N, and S, and wherein the nitrogen and sulfur atoms may be optionally oxidized and the nitrogen heteroatom may be optionally quaternized. The heteroatom(s) may be placed at any position of the heteroalkyl group, including between the rest of the heteroalkyl group and the fragment to which it is attached, as well as attached to the most distal carbon atom in the heteroalkyl group. Examples include: —O—CH₂—CH₂—CH₃, —CH₂—CH₂—CH₂—OH, —CH₂—CH₂—NH—CH₃, —CH₂—S—CH₂—CH₃, and —CH₂CH₂—S(═O)—CH₃. Up to two heteroatoms may be consecutive, such as, for example, —CH₂—NH—OCH₃, or —CH₂—CH₂—S—S—CH₃.

As used herein, the term “heteroalkenyl” by itself or in combination with another term refers to, unless otherwise stated, a stable straight or branched chain monounsaturated or diunsaturated hydrocarbon group consisting of the stated number of carbon atoms and one or two heteroatoms selected from the group consisting of O, N, and S, and wherein the nitrogen and sulfur atoms may optionally be oxidized and the nitrogen heteroatom may optionally be quaternized. Up to two heteroatoms may be placed consecutively. Examples include —CH═CH—O—CH₃, —CH═CH—CH₂—OH, —CH₂—CH═N—OCH₃, —CH═CH—N(CH₃)—CH₃, and —CH₂—CH═CH—CH₂—SH.

As used herein, the term “heteroalkyl” by itself or in combination with another term refers to, unless otherwise stated, a stable straight or branched chain alkyl group consisting of the stated number of carbon atoms and one or two heteroatoms selected from the group consisting of O, N. and S, and wherein the nitrogen and sulfur atoms may be optionally oxidized and the nitrogen heteroatom may be optionally quaternized. The heteroatom(s) may be placed at any position of the heteroalkyl group, including between the rest of the heteroalkyl group and the fragment to which it is attached, as well as attached to the most distal carbon atom in the heteroalkyl group. Examples include: —OCH₂CH₂CH₃, —CH₂CH₂CH₂OH, —CH₂CH₂NHCH₃, —CH₂SCH₂CH₃, and —CH₂CH₂S(═O)CH₃. Up to two heteroatoms may be consecutive, such as, for example, —CH₂NH—OCH₃, or —CH₂CH₂SSCH₃.

As used herein, the term “heteroaryl” or “heteroaromatic” refers to a heterocycle having aromatic character. A polycyclic heteroaryl may include one or more rings that are partially saturated. Examples include tetrahydroquinoline and 2,3-dihydrobenzofuryl.

As used herein, the term “heterocycle” or “heterocyclyl” or “heterocyclic” by itself or as part of another substituent refers to, unless otherwise stated, an unsubstituted or substituted, stable, mono- or multi-cyclic heterocyclic ring system that comprises carbon atoms and at least one heteroatom selected from the group consisting of N, O, and S, and wherein the nitrogen and sulfur heteroatoms may be optionally oxidized, and the nitrogen atom may be optionally quaternized. The heterocyclic system may be attached, unless otherwise stated, at any heteroatom or carbon atom that affords a stable structure. A heterocycle may be aromatic or non-aromatic in nature. In certain embodiments, the heterocycle is a heteroaryl.

Examples of non-aromatic heterocycles include monocyclic groups such as aziridine, oxirane, thiirane, azetidine, oxetane, thietane, pyrrolidine, pyrroline, imidazoline, pyrazolidine, dioxolane, sulfolane, 2,3-dihydrofuran, 2,5-dihydrofuran, tetrahydrofuran, thiophane, piperidine, 1,2,3,6-tetrahydropyridine, 1,4-dihydropyridine, piperazine, morpholine, thiomorpholine, pyran, 2,3-dihydropyran, tetrahydropyran, 1,4-dioxane. 1,3-dioxane, homopiperazine, homopiperidine, 1,3-dioxepane, 4,7-dihydro-1,3-dioxepin and hexamethyleneoxide.

Examples of heteroaryl groups include pyridyl, pyrazinyl, pyrimidinyl (such as, but not limited to, 2- and 4-pyrimidinyl), pyridazinyl, thienyl, furyl, pyrrolyl, imidazolyl, thiazolyl, oxazolyl, pyrazolyl, isothiazolyl, 1,2,3-triazolyl, 1,2,4-triazolyl, 1,3,4-triazolyl, tetrazolyl, 1,2,3-thiadiazolyl, 1,2,3-oxadiazolyl, 1,3,4-thiadiazolyl and 1,3,4-oxadiazolyl.

Examples of polycyclic heterocycles include indolyl (such as, but not limited to, 3-, 4-5-, 6- and 7-indolyl), indolinyl, quinolyl, tetrahydroquinolyl, isoquinolyl (such as, but not limited to, 1- and 5-isoquinolyl), 1,2,3,4-tetrahydroisoquinolyl, cinnolinyl, quinoxalinyl (such as, but not limited to, 2- and 5-quinoxalinyl), quinazolinyl, phthalazinyl, 1,8-naphthyridinyl, 1,4-benzodioxanyl, coumarin, dihydrocoumarin, 1,5-naphthyridinyl, benzofuryl (such as, but not limited to, 3-, 4-. 5-, 6- and 7-benzofuryl). 2,3-dihydrobenzofuryl, 1,2-benzisoxazolyl, benzothienyl (such as, but not limited to, 3-, 4-, 5-, 6-, and 7-benzothienyl), benzoxazolyl, benzothiazolyl (such as, but not limited to, 2-benzothiazolyl and 5-benzothiazolyl), purinyl, benzimidazolyl, benztriazolyl, thioxanthinyl, carbazolyl, carbolinyl, acridinyl, pyrrolizidinyl, and quinolizidinyl.

The aforementioned listing of heterocyclyl and heteroaryl moieties is intended to be representative and not limiting.

“Identity” as used herein refers to the subunit sequence identity between two polymeric molecules particularly between two amino acid molecules, such as, between two polypeptide molecules. When two amino acid sequences have the same residues at the same positions; e.g., if a position in each of two polypeptide molecules is occupied by an arginine, then they are identical at that position. The identity or extent to which two amino acid sequences have the same residues at the same positions in an alignment is often expressed as a percentage. The identity between two amino acid sequences is a direct function of the number of matching or identical positions: e.g., if half (e.g., five positions in a polymer ten amino acids in length) of the positions in two sequences are identical, the two sequences are 50% identical; if 90% of the positions (e.g., 9 of 10), are matched or identical, the two amino acids sequences are 90% identical.

The term “immune response” as used herein is defined as a cellular response to an antigen that occurs when lymphocytes identify antigenic molecules as foreign and induce the formation of antibodies and/or activate lymphocytes to remove the antigen.

The term “immunosuppressive” is used herein to refer to reducing overall immune response.

“Isolated” means altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell.

The term “linker” as used herein refers to a divalent, chemically inert group which serves to link two chemical species

By the term “modified” as used herein, is meant a changed state or structure of a molecule or cell of the invention. Molecules may be modified in many ways, including chemically, structurally, and functionally. Cells may be modified through the introduction of nucleic acids.

By the term “modulating,” as used herein, is meant mediating a detectable increase or decrease in the level of a response in a subject compared with the level of a response in the subject in the absence of a treatment or compound, and/or compared with the level of a response in an otherwise identical but untreated subject. The term encompasses perturbing and/or affecting a native signal or response thereby mediating a beneficial therapeutic response in a subject, preferably, a human.

In the context of the present invention, the following abbreviations for the commonly occurring nucleic acid bases are used. “A” refers to adenosine, “C” refers to cytosine, “G” refers to guanosine, “T” refers to thymidine, and “U” refers to uridine.

The term “oligonucleotide” typically refers to short polynucleotides. It will be understood that when a nucleotide sequence is represented by a DNA sequence (i.e., A, T, C, G), this also includes an RNA sequence (i.e., A, U, C, G) in which “U” replaces “T.”

Unless otherwise specified, a “nucleotide sequence encoding an amino acid sequence” includes all nucleotide sequences that are degenerate versions of each other and that encode the same amino acid sequence. A nucleotide sequence that encodes a protein or an RNA may also include introns to the extent that the nucleotide sequence encoding the protein may in some version contain an intron(s).

“Parenteral” administration of an immunogenic composition includes, e.g., subcutaneous (s.c.), intravenous (i.v.), intramuscular (i.m.), or intrasternal injection, or infusion techniques.

The term “polynucleotide” as used herein is defined as a chain of nucleotides. Furthermore, nucleic acids are polymers of nucleotides. Thus, nucleic acids and polynucleotides as used herein are interchangeable. One skilled in the art has the general knowledge that nucleic acids are polynucleotides, which can be hydrolyzed into the monomeric “nucleotides.” The monomeric nucleotides can be hydrolyzed into nucleosides. As used herein polynucleotides include, but are not limited to, all nucleic acid sequences which are obtained by any means available in the art, including, without limitation, recombinant means, i.e., the cloning of nucleic acid sequences from a recombinant library or a cell genome, using ordinary cloning technology and PCR, and the like, and by synthetic means.

As used herein, the terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

By the term “specifically binds,” as used herein with respect to an antibody or antigen binding fragment thereof, is meant an antibody or antigen binding fragment thereof which recognizes a specific antigen, but does not substantially recognize or bind other molecules in a sample. For example, an antibody that specifically binds to an antigen from one species may also bind to that antigen from one or more species. But, such cross-species reactivity does not itself alter the classification of an antibody as specific. In another example, an antibody that specifically binds to an antigen may also bind to different allelic forms of the antigen. However, such cross reactivity does not itself alter the classification of an antibody as specific. In some instances, the terms “specific binding” or “specifically binding,” can be used in reference to the interaction of an antibody, a protein, or a peptide with a second chemical species, to mean that the interaction is dependent upon the presence of a particular structure (e.g., an antigenic determinant or epitope) on the chemical species; for example, an antibody recognizes and binds to a specific protein structure rather than to proteins generally. If an antibody is specific for epitope “A”, the presence of a molecule containing epitope A (or free, unlabeled A), in a reaction containing labeled “A” and the antibody, will reduce the amount of labeled A bound to the antibody.

By the term “stimulation,” is meant a primary response induced by binding of a stimulatory molecule (e.g., a TCR/CD3 complex) with its cognate ligand thereby mediating a signal transduction event, such as, but not limited to, signal transduction via the TCR/CD3 complex. Stimulation can mediate altered expression of certain molecules, such as upregulation of IFNγ, and/or reorganization of cytoskeletal structures, and the like.

A “stimulatory molecule,” as the term is used herein, means a molecule on a T cell that specifically binds with a cognate stimulatory ligand present on an antigen presenting cell.

A “stimulatory ligand,” as used herein, means a ligand that when present on an antigen presenting cell (e.g., an aAPC, a dendritic cell, a B-cell, and the like) can specifically bind with a cognate binding partner (referred to herein as a “stimulatory molecule”) on a T cell, thereby mediating a primary response by the T cell, including, but not limited to, activation, initiation of an immune response, proliferation, and the like. Stimulatory ligands are well-known in the art and encompass, inter alia, an MHC Class I molecule loaded with a peptide, an anti-CD3 antibody, a superagonist anti-CD28 antibody, and a superagonist anti-CD2 antibody.

The term “subject” is intended to include living organisms in which an immune response can be elicited (e.g., mammals). A “subject” or “patient,” as used therein, may be a human or non-human mammal. Non-human mammals include, for example, livestock and pets, such as ovine, bovine, porcine, canine, feline and murine mammals. Preferably, the subject is human.

As used herein, the term “substituted alkyl,” “substituted cycloalkyl,” “substituted alkenyl,” “substituted alkynyl,” “substituted heteroalkyl,” “substituted alkylene,” or “substituted heteroalkylene” refers to alkyl, cycloalkyl, alkenyl, alkynyl, heteroalkyl, alkylenyl, or heteroalkylenyl, as defined elsewhere herein, substituted by one, two or three substituents independently selected from the group consisting of halogen, —OH, alkoxy, tetrahydro-2-H-pyranyl, —NH₂, —NH(C₁-C₆ alkyl), —N(C₁-C₆ alkyl)₂, 1-methyl-imidazol-2-yl, pyridin-2-yl, pyridin-3-yl, pyridin-4-yl, —C(═O)OH, —C(═O)O(C₁-C₆)alkyl, trifluoromethyl, —C≡N, —C(═O)NH₂, —C(═O)NH(C₁-C₆)alkyl, —C(═O)N((C₁-C₆)alkyl)₂, —SO₂NH₂, —SO₂NH(C₁-C₆ alkyl), —SO₂N(C₁-C₆ alkyl)₂, —C(═NH)NH₂, and —NO₂, in certain embodiments containing one or two substituents independently selected from halogen, —OH, alkoxy, —NH₂, trifluoromethyl, —N(CH₃)₂, and —C(═O)OH, in certain embodiments independently selected from halogen, alkoxy and —OH. Examples of substituted alkyls include, but are not limited to, 2,2-difluoropropyl, 2-carboxycyclopentyl and 3-chloropropyl.

For aryl, aryl-(C₁-C₃)alkyl and heterocyclyl groups, the term “substituted” as applied to the rings of these groups refers to any level of substitution, namely mono-, di-, tri-, tetra-, or penta-substitution, where such substitution is permitted. The substituents are independently selected, and substitution may be at any chemically accessible position. In certain embodiments, the substituents vary in number between one and four. In other embodiments, the substituents vary in number between one and three. In yet another embodiments, the substituents vary in number between one and two. In yet other embodiments, the substituents are independently selected from the group consisting of C₁-C₆ alkyl, —OH, C₁-C₆ alkoxy, halo, amino, acetamido and nitro. As used herein, where a substituent is an alkyl or alkoxy group, the carbon chain may be branched, straight or cyclic.

In certain embodiments, each occurrence of alkyl or cycloalkyl is independently optionally substituted with at least one substituent selected from the group consisting of C₁-C₆ alkyl, halo, —OR, phenyl (thus yielding, in non-limiting examples, optionally substituted phenyl-(C₁-C₃ alkyl), such as, but not limited to, benzyl or substituted benzyl) and —N(R)(R), wherein each occurrence of R is independently H. C₁-C₆ alkyl or C₃-C₈ cycloalkyl. In other embodiments, each occurrence of aryl or heteroaryl is independently optionally substituted with at least one substituent selected from the group consisting of C₁-C₆ alkyl, C₁-C₆ haloalkyl, C₁-C₆ haloalkoxy, halo, —CN, —OR, —N(R)(R), —NO₂, —S(═O)₂N(R)(R), acyl, and C₁-C₆ alkoxycarbonyl, wherein each occurrence of R is independently H, C₁-C₆ alkyl or C₃-C₈ cycloalkyl. In yet other embodiments, each occurrence of aryl or heteroaryl is independently optionally substituted with at least one substituent selected from the group consisting of C₁-C₆ alkyl, C₁-C₆ haloalkyl, C₁-C₆ haloalkoxy, halo, —CN, —OR, —N(R)(R), and C₁-C₆ alkoxycarbonyl, wherein each occurrence of R is independently H, C₁-C₆ alkyl or C₃-C₈ cycloalkyl.

Unless otherwise noted, when two substituents are taken together to form a ring having a specified number of ring atoms (e.g., R² and R³ taken together with the nitrogen to which they are attached to form a ring having from 3 to 7 ring members), the ring can have carbon atoms and optionally one or more (e.g., 1 to 3) additional heteroatoms independently selected from nitrogen, oxygen, or sulfur. The ring can be saturated or partially saturated, and can be optionally substituted.

Whenever a term or either of their prefix roots appear in a name of a substituent the name is to be interpreted as including those limitations provided herein. For example, whenever the term “alkyl” or “aryl” or either of their prefix roots appear in a name of a substituent (e.g., arylalkyl, alkylamino) the name is to be interpreted as including those limitations given elsewhere herein for “alkyl” and “aryl” respectively.

In certain embodiments, substituents of compounds are disclosed in groups or in ranges. It is specifically intended that the description include each and every individual subcombination of the members of such groups and ranges. For example, the term “C₁₋₆ alkyl” is specifically intended to individually disclose C₁, C₂, C₃, C₄, C₅, C₆, C₁-C₆, C₁-C₅, C₁-C₄, C₁-C₃, C₁-C₂, C₂-C₆, C₂-C₅, C₂-C₄, C₂-C₃, C₃-C₆, C₃-C₅, C₃-C₄, C₄-C₆, C₄-C₅, and C₅-C₆ alkyl.

A “target site” or “target sequence” refers to a nucleic acid sequence that defines a portion of a nucleic acid to which a binding molecule may specifically bind under conditions sufficient for binding to occur. In some embodiments, a target sequence refers to a genomic nucleic acid sequence that defines a portion of a nucleic acid to which a binding molecule may specifically bind under conditions sufficient for binding to occur.

As used herein, the term “T cell receptor” or “TCR” refers to a complex of membrane proteins that participate in the activation of T cells in response to the presentation of antigen.

The TCR is responsible for recognizing antigens bound to major histocompatibility complex molecules. TCR is composed of a heterodimer of an alpha (α) and beta (β) chain, although in some cells the TCR consists of gamma and delta (γ/δ) chains. TCRs may exist in alpha/beta and gamma/delta forms, which are structurally similar but have distinct anatomical locations and functions. Each chain is composed of two extracellular domains, a variable and constant domain. In some embodiments, the TCR may be modified on any cell comprising a TCR, including, for example, a helper T cell, a cytotoxic T cell, a memory T cell, regulatory T cell, natural killer T cell, and gamma delta T cell.

The term “therapeutic” as used herein means a treatment and/or prophylaxis. A therapeutic effect is obtained by suppression, remission, or eradication of a disease state.

The term “transfected” or “transformed” or “transduced” as used herein refers to a process by which exogenous nucleic acid is transferred or introduced into the host cell. A “transfected” or “transformed” or “transduced” cell is one which has been transfected, transformed or transduced with exogenous nucleic acid. The cell includes the primary subject cell and its progeny.

To “treat” a disease as the term is used herein, means to reduce the frequency or severity of at least one sign or symptom of a disease or disorder experienced by a subject.

A “vector” is a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer of nucleic acid into cells, such as, for example, polylysine compounds, liposomes, and the like. Examples of viral vectors include, but are not limited to, Sendai viral vectors, adenoviral vectors, adeno-associated virus vectors, retroviral vectors, lentiviral vectors, and the like.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### Compositions

In one aspect, the present disclosure provides a disruptable linker comprising a compound of formula (I):

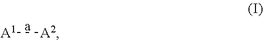

wherein:
A¹ is

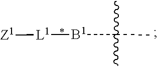

A² is

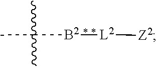

Z¹ and Z² each independently comprise a cross-linking moiety;
L¹ and L² each independently comprise a linker;
B¹ comprises β-cyclodextrin (β-CD);
B² comprises a β-CD binding moiety;
indicates the bond between L¹ and B¹;
* indicates the bond between L² and B²; and
bond a is an optional non-covalent bonding interaction.

In certain embodiments, bond a is present. In certain embodiments, bond a is absent. In certain embodiments, bond a is present and may be disrupted (i.e., converted from absent to present). In certain embodiments, bond a is absent and may be formed.

In certain embodiments, bond a is absent and A¹ is

$${Z}^{1} - {L}^{1} {-}^{*} {B}^{1} .$$

In certain embodiments, bond a is absent and

$${A}^{2} is {B}^{2} {-}^{**} {L}^{2} - {Z}^{2} .$$

In certain embodiments, the compound of formula (I) is

$${Z}^{1} - {L}^{1} {-}^{*} {B}^{1} {-- -}^{a} {A}^{2} .$$

In certain embodiments, the compound of formula (I) is

$${A}^{1} {-- -}^{a} {B}^{2} {-}^{**} {L}^{2} - {Z}^{2} .$$

In certain embodiments, the compound of formula (I) is a compound of formula (Ia):

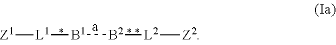

In certain embodiments, Z¹ and Z² are each independently selected from the group consisting of

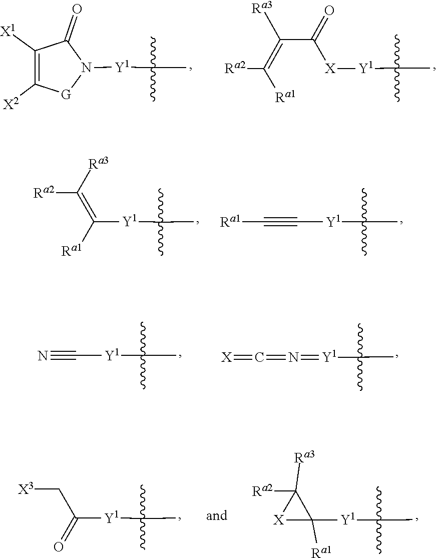

wherein:
X is selected from the group consisting of O, S, and N(R^{A});
X¹ and X² are each independently selected from the group consisting of H and halogen;
G is optionally substituted C₁-C₂ alkylene or —C(═O)—;
X³ is selected from the group consisting of I, Br, Cl, and OR^{D};
Y¹ is selected from the group consisting of a bond, —C(═O)—, —C(═O)(C₁-C₆ alkylene)-, —C(═O)(C₁-C₆ heteroalkylene)-, —C(═O)(C₁-C₆ alkylene)C(═O)—, and —C(═O)(C₁-C₆ heteroalkylene)C(═O)—;
each occurrence of R^{a1}, R^{a2}, and R^{a3}, if present, is independently selected from the group consisting of H, halogen, CN, NO₂, optionally substituted C₁-C₆ alkyl, optionally substituted C₃-C₈ cycloalkyl, optionally substituted C₁-C₆ alkoxy, optionally substituted C₃-C₈ cycloalkoxy, heterocyclyl, phenyl, naphthyl, heteroaryl, OR^{B}, N(R^{B})(R^{C}), NO₂, C(═O)N(R^{B})(R^{C}), C(═O)R^{B}, —C(═O)OR^{B}, OC(═O)R^{B}, OC(═O)OR^{B}, SR^{B}, S(═O)R^{B}, S(═O)₂R^{B}, N(R^{B})S(═O)₂R^{C}, N(R^{B})C(═O)R^{C}, and S(═O)₂N(R^{B})(R^{C});
each occurrence of R^{A}, R^{B}, and R^{C} is independently selected from the group consisting of H, —C(═O)(C₁-C₆ alkyl), —C(═O)(C₁-C₆ haloalkyl), optionally substituted C₁-C₆ alkyl, optionally substituted C₁-C₃ haloalkyl, optionally substituted C₃-C₈ cycloalkyl, optionally substituted C₁-C₆ alkoxy, and optionally substituted C₃-C₈ cycloalkoxy, optionally substituted phenyl, optionally substituted naphthyl, and optionally substituted heteroaryl; and
R^{D} is selected from the group consisting of H, —C(═O)R^{B}, and —S(═O)₂R^{B}.

In certain embodiments, Z¹ is

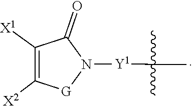

In certain embodiments, Z¹ is

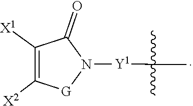

In certain embodiments, Y¹ is —C(═O)(CH₂CH₂)—. In certain embodiments, G is —C(═O).

In certain embodiments, X¹ is H. In certain embodiments, X² is H. In certain embodiments, X¹ and X² are both H.

In certain embodiments, Z¹ is

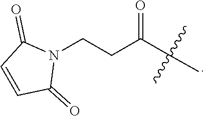 [SMILES: *C(=O)CCN1C(=O)C=CC1=O]

In certain embodiments, Z² is

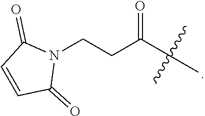 [SMILES: *C(=O)CCN1C(=O)C=CC1=O]

In certain embodiments. Z¹ and Z² are both

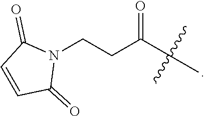 [SMILES: *C(=O)CCN1C(=O)C=CC1=O]

In certain embodiments, L¹ is selected from the group consisting of a bond, optionally substituted C₁-C₁₂ alkylene, and optionally substituted C₁-C₁₂heteroalkylene. In certain embodiments, L² is selected from the group consisting of a bond, optionally substituted C₁-C₁₂ alkylene, and optionally substituted C₁-C₁₂heteroalkylene.

In certain embodiments, L¹ is *—NH(CH₂CH₂)(OCH₂CH₂)ₙNH—, wherein n is selected from the group consisting of 1, 2, 3, and 4. In certain embodiments, L¹⁼² is —NH(CH₂CH₂)(OCH₂CH₂)ₙNH—, wherein n is selected from the group consisting of 1, 2, 3, and 4. In certain embodiments, n is 1. In certain embodiments, n is 2. In certain embodiments, n is 3. In certain embodiments, n is 4.

In certain embodiments, L¹ is *—NH(CH₂CH₂)(OCH₂CH₂)ₙNH— and L² is **—NH(CH₂CH₂)(OCH₂CH₂)ₙNH—.

In certain embodiments, the bond indicated as * comprises a covalent bond between L¹ and an exocyclic methylene of the β-CD in B¹. In certain embodiments, the bond indicated as * comprises a C—N bond. In certain embodiments, the bond is formed by displacement of a tosylated primary hydroxyl of a glucose monomer of the β-CD in B¹. In certain embodiments, the bond indicated as * is formed by displacement of a tosylate by an amine in L¹.

In certain embodiments, B² is

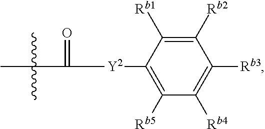

wherein:
Y² is selected from the group consisting of a bond, C₁-C₆ alkylene, and C₁-C₆ heteroalkylene; and
each occurrence of R^{b1}, R^{b2}, R^{b3}, R^{b4}, and R^{b5} if present, is independently selected from the group consisting of H, optionally substituted C₁-C₆ alkyl, optionally substituted C₃-C₈ cycloalkyl, optionally substituted C₁-C₆ alkoxy, optionally substituted C₃-C₈ cycloalkoxy, optionally substituted C₂-C₈ heterocycloalkyl, optionally substituted phenyl, optionally substituted naphthyl, and optionally substituted heteroaryl.

In certain embodiments, Y² is —CH₂CH₂—.

In certain embodiments, at least one of R^{b1}, R^{b2}, R^{b3}, R^{b4}, and R^{b5} is H. In certain embodiments, at least two of R^{b1}, R^{b2}, R^{b3}, R^{b4}, and R^{b5} are H. In certain embodiments, at least three of R^{b1}, R^{b2}, R^{b3}. R^{b4}, and R^{b5} are H. In certain embodiments, at least four of R^{b1}, R^{b2}, R^{b3}, R^{b4}, and R^{b5} are H. In certain embodiments, each of R^{b1}, R^{b2}, R^{b3}, R^{b4}, and R^{b5} are H.

In certain embodiments, B² is

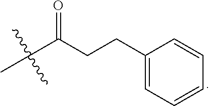 [SMILES: *C(=O)CCc1ccccc1]

In certain embodiments, A¹ is

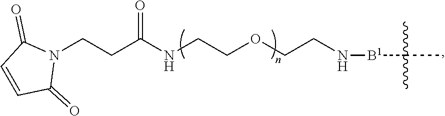

wherein n is selected from the group consisting of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, and 50.

In certain embodiments, A² is

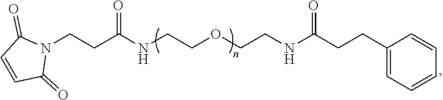

wherein each occurrence of n is independently selected from the group consisting of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, and 50.

In certain embodiments, the non-covalent bonding interaction of bond a is disrupted with a disrupting agent. In certain embodiments, the disrupting agent is amantadine. In certain embodiments, the disrupting agent is an amino acid. In certain embodiments, the amino acid comprises an aromatic moiety. In certain embodiments, the amino acid comprising an aromatic moiety is phenylalanine. In certain embodiments, the amino acid comprising an aromatic moiety is tyrosine.

### Bispecific Antibodies and Bispecific T Cell Nanoengagers (BiTEs)

In one aspect, the present disclosure provides a switchable Bispecific T cell Engager (switch-BiTE). In certain embodiments, the switch-BiTE comprises a first binding domain specific for a surface antigen on a target cell. In certain embodiments, the switch-BiTE comprises a second binding domain specific for a surface antigen on an immune effector cell. In certain embodiments, the switch-BiTE comprises the disruptable linker of the present disclosure. In certain embodiments, bond a is present. In certain embodiments, the cross-linking moiety in Z¹ is covalently bonded to the first binding domain and the cross-linking moiety in Z² is covalently bonded to the second binding domain. In certain embodiments, the cross-linking moiety in Z¹ is covalently bonded to the second binding domain and the cross-linking moiety in Z² is covalently bonded to the first binding domain.

In certain embodiments, the first binding domain is selected from the group consisting of a Fab, a single-chain variable fragment (scFv), a single-domain antibody, a full-length antibody and a receptor ligand. In certain embodiments, the first binding domain is a Fab. In certain embodiments, the surface antigen on the target cell is a tumor-associated antigen. In certain embodiments, the tumor-associated antigen is selected from the group consisting of BCMA, C-Met, CD19, CD20, CEA, EGFR, EphA2, HER2, MART1, Mesothelin, MUC1, NY-ESO-1, PD-L1, PSCA, PSMA, ROR1, VEGFR2. In certain embodiments, the surface antigen on the target cell is HER2. In certain embodiments, target cell is a tumor cell. In certain embodiments, the second binding domain is selected from the group consisting of consisting of a Fab, a single-chain variable fragment (scFv), a full-length antibody, and a single-domain antibody. In certain embodiments, the second binding domain is a Fab. In certain embodiments, the immune effector cell is a T cell. In certain embodiments, the second domain is specific for CD3. In certain embodiments, the second domain is specific for CD3ε.

In certain embodiments, the disruptable linker comprises Phenyl-PEG-MAL and β-CD-PEG-MAL. In certain embodiments, bond a of the disruptable linker is disrupted with a disrupting agent. In certain embodiments, the disrupting agent has a higher affinity for β-CD than does Phenyl-PEG-MAL. In certain embodiments, the disrupting agent is amantadine. In certain embodiments, the disrupting agent is an amino acid. In certain embodiments, the amino acid is an amino acid comprising an aromatic moiety. In certain embodiments, the amino acid comprising an aromatic moiety is phenylalanine or tyrosine.

Certain embodiments of the invention include bispecific antibodies, bispecific T cell nanoengagers (BiTEs or BiTNEs), or fragments thereof. A bispecific antibody comprises two or more different antigen binding domains with differing binding specificities which thus bind to two different antigens. In one embodiment, the bispecific antibody comprises one or more first antigen binding domains that bind to a first antigen and one or more second antigen binding domains that bind to a second antigen attached by a linker. Bispecific T cell nanoengagers or BiTEs or BiTNEs are bispecific antibodies in which one of the antigen-binding domains is specific for an antigen expressed by a T cell such that binding of the BiTE or BiTNE to the T cell induces its activation.

In certain embodiments, the linker attaching the two antigen-binding domains is a disruptable linker comprising a β-cyclodextrin (β-CD) moiety and a β-CD binding moiety, such that the introduction of a small molecule can disrupt the linker and separate the antigen-binding domains, thus disrupting and inactivating the bispecific antibody or BiTE/BiTNE. In this way, the bispecific antibody or BiTE is “switchable” in that it's function can be deactivated at will, for example in order to limit any kind of toxicity associated with the in vivo action of the bispecific antibody or BiTE/BiTNE. As such, the disruptable bispecific antibodies or BiTEs of the invention are also known as switch-BiTEs or switch-BiTEs. As used herein, the terms BiTE and switch-BiTE and BiTNE and switch-BiTE are used interchangeably.

In certain embodiments, the BiTE or switch-BiTE of the invention comprises a first and second antigen binding domains bind an antigen on a target cell and an antigen on a T cell. In another embodiment, the first antigen binding domain is specific for at least one antigen on a target cell and the second antigen binding fragment is specific for an antigen on an effector T cell. Examples of effector T cell antigens that can be bound by the BiTEs or switch-BiTEs of the invention include but are not limited to CD3, CD4, CD8, or the TCR. By the term “effector T cell” is meant any T cell type that actively responds to a stimulus. Examples of effector T cells include CD4⁺ and CD8⁺ T cells, helper T cells including Th¹. Th², and Th¹⁷, among others, and cytotoxic T cells, also referred to as Tc cells, CTLs, T-killer cells, and killer T cells, among others.

In another embodiment, the first and second antigen-binding domains bind an antigen on a target cell and an antigen on a T cell. For example, the T cell antigen can include CD3. CD4, CD8, TCRα, TCRβ, TCRγ, TCRδ, or a combination thereof. In another embodiment, the bispecific antibody comprises bispecificity for an antigen on the target cell and the CD3 on the T cell. The binding of the T cell-specific binding domain of the BiTE activates the function of the T cell and the simultaneous binding of the target cell directs the T cells function to interact with the target cell. In one nonlimiting example, the T cell is cytotoxic T cell and the binding of the BiTE to an antigen on the cytotoxic T cell and an antigen on a target cell results in the killing of the target cell. In another nonlimiting example, the simultaneous binding of the T cell and the target cell activates other functions of the T cell including, but not limited to, proliferation of the T cell and the production of pro-inflammatory signaling molecules including cytokines, chemokines, and the like.

In certain aspects, the invention includes the use of BiTEs to induce immune responses against the target cells beyond the binding of the BiTE to the target call and T cell. In certain embodiments, the cell lysis induced by the BiTEs releases target cell antigens and pro-inflammatory molecules, also known as damage-associated pattern molecules or DAMPs which in combination with chemokines and cytokines released by the T cell prime the immune system against the target cells. This process results in both T cell and antibody responses against target cell antigens that do not necessarily need the continued function of the BiTE molecules. As such, the BiTEs of the invention can be used as in situ vaccines against target cell antigens (e.g. tumor cell antigens).

However, the present invention is not limited by the use of any particular antibody or antigen-binding domain derived therefrom. Rather, any antigen-binding domain or antibody can be used. Examples of target cell associated antigens are described elsewhere herein, all of which may be targeted by the BiTEs of the present invention.

### T Cell Antigens

In certain embodiments, the activating T cell antigen is CD3, CD4, CD8, the T cell receptor (TCR), or any fragment thereof. In certain embodiments, the antigen-binding domain specifically binds to a subunit of the CD3 complex, particularly a signal-transducing subunit which activates the T cell. Examples of such activating subunits that can be bound by the BiTEs or BiTNEs of the invention include but are not limited to CD3γ, CD3δ, CD3ε, CD3ζ, or any combination thereof. In certain embodiments, the antigen-binding domain specific for a T cell antigen is CD3ε. Here, the BiTE or BiTNE molecule comprises an antibody, such as a synthetic antibody, human antibody, a humanized antibody, single chain variable fragment, single domain antibody, an antigen binding fragment thereof, and any combination thereof, that specifically binds to the activating T cell antigen. Examples of the activating T cell antigen may include anti-CD3, anti-CD4, anti-CD8, anti-TCR, and fragments thereof.

### Target Cell Antigens

In certain embodiments, the bispecific antibody or BiTE comprises specificity to a target cell antigen. The target cell antigen may include the same target cell antigen that the T cell receptor binds or may include a different target cell antigen. The target cell antigen may include any type of ligand that defines the target cell. For example, the target cell antigen may be chosen to recognize a ligand that acts as a cell marker on target cells associated with a particular disease state. Thus examples of cell markers that may act as ligands for the antigen moiety domain in a BiTE molecule, include those associated with viral, bacterial and parasitic infections, autoimmune disease, cancer cells, as well as damaged or diseased tissues associated with, for example, cardiovascular disease and fibrosis among other conditions.

In one embodiment, the target cell antigen includes any tumor associated antigen (TAA) and viral antigen, or any fragment thereof. In this embodiment, the BiTE molecule comprises an antibody, such as a synthetic antibody, human antibody, a humanized antibody, single chain variable fragment, single domain antibody, an antigen binding fragment thereof, and any combination thereof, that specifically binds to the target cell antigen.

Tumor associated antigens (TAAs) or tumor antigens are proteins that are produced by tumor cells that elicit an immune response, particularly T-cell mediated immune responses. The selection of the antigen binding domain of the invention will depend on the particular type of cancer to be treated. Tumor antigens are well known in the art and include, for example, a glioma-associated antigen, carcinoembryonic antigen (CEA), β-human chorionic gonadotropin, alphafetoprotein (AFP), lectin-reactive AFP, thyroglobulin, RAGE-1, MN-CA IX, human telomerase reverse transcriptase, RU1, RU2 (AS), intestinal carboxyl esterase, mut hsp70-2, M-CSF, prostase, prostate-specific antigen (PSA), PAP, NY-ESO-1, LAGE-1a, p53, prostein, PSMA, Her2/neu, survivin and telomerase, prostate-carcinoma tumor antigen-1 (PCTA-1), MAGE, ELF2M, neutrophil elastase, ephrinB2, CD22, insulin growth factor (IGF)-I, IGF-II, IGF-I receptor and mesothelin.

In certain embodiments, the tumor antigen comprises one or more cancer epitopes associated with malignancy. Malignant tumors express a number of proteins that can serve as target antigens for immune targeting. These molecules include but are not limited to tissue-specific antigens such as MART-1, tyrosinase and GP100 in melanoma and prostatic acid phosphatase (PAP) and prostate-specific antigen (PSA) in prostate cancer. Other target molecules belong to the group of transformation-related molecules such as the oncogene HER-2/Neu/ErbB-2. Yet another group of target antigens are onco-fetal antigens such as carcinoembryonic antigen (CEA). In B-cell lymphoma the tumor-specific idiotype immunoglobulin constitutes a truly tumor-specific immunoglobulin antigen that is unique to the individual tumor. B-cell differentiation antigens such as CD19, CD20 and CD37 are other candidates for target antigens in B-cell lymphoma. Some of these antigens (CEA, HER-2, CD19, CD20, idiotype) have been used as targets for passive immunotherapy with monoclonal antibodies or with chimeric antigen receptor (CAR) expressing T cells.

The type of tumor antigen targeted by the bispecific antibodies or BiTEs of the invention may also be a tumor-specific antigen (TSA) or a tumor-associated antigen (TAA). Non-limiting examples of TSA or TAA antigens include the following: Differentiation antigens such as MART-1/MelanA (MART-I), gpl00 (Pmel 17), tyrosinase, TRP-1, TRP-2 and tumor-specific multilineage antigens such as MAGE-1, MAGE-3, BAGE, GAGE-1, GAGE-2, p15; overexpressed embryonic antigens such as CEA; overexpressed oncogenes and mutated tumor-suppressor genes such as p53, Ras, HER-2/neu: unique tumor antigens resulting from chromosomal translocations: such as BCR-ABL. E2A-PRL, H4-RET, IGH-IGK, MYL-RAR; and viral antigens, such as the Epstein Barr virus antigens EBVA and the human papillomavirus (HPV) antigens E6 and E7. Other large, protein-based antigens include TSP-180, MAGE-4, MAGE-5, MAGE-6, RAGE, NY-ESO, p185erbB2, p180erbB-3, c-met, nm-23H1. PSA. TAG-72. CA 19-9, CA 72-4, CAM 17.1, NuMa, K-ras, beta-Catenin, CDK4, Mum-1, p 15, p 16, 43-9F, 5T4, 791Tgp72, alpha-fetoprotein, beta-HCG, BCA225, BTAA, CA 125, CA 15-3\CA 27.29\BCAA, CA 195, CA 242, CA-50, CAM43, CD68\P1, CO-029, FGF-5, G250, Ga733\EpCAM, HTgp-175, M344, MA-50, MG7-Ag, MOV18, NB/70K, NY-CO-1, RCAS1, SDCCAG16, TA-90\Mac-2 binding protein\cyclophilin C-associated protein, TAAL6, TAG72, TLP, and TPS.

### Antigen Binding Domains

The antigen binding domains of a bispecific antibody or BiTE are a regions for binding to a specific target antigen including proteins, carbohydrates, and glycolipids. In some embodiments, the bispecific antibody or BiTE comprises affinity to a target antigen (e.g. a tumor associated antigen) on a target cell (e.g. a cancer cell) and/or a T cell. The target antigen may include any type of protein, or epitope thereof, associated with the target cell and/or T cell.

The antigen binding domain can include any domain that binds to the antigen and may include, but is not limited to, a monoclonal antibody, a polyclonal antibody, a synthetic antibody, a human antibody, a humanized antibody, a non-human antibody, and any fragment thereof.

It is contemplated that the antigen-binding fragments of the invention can be full length antibodies or antigen-binding fragments thereof, or synthetic antibodies such as single-chain antigen-binding fragments (scFv), single-domain antibodies, a Fab, a F(ab′)₂, a (scFv)₂, or any combination thereof. In certain embodiments, the antigen-binding fragment is a Fab or antigen-binding fragment derived from a full-length antibody. In certain embodiments, the Fabs can be obtained by enzymatic digestion of full length antibodies. In one nonlimiting example, the enzyme papain is used to cleave the two Fab fragments from the constant region of an antibody molecule, thus producing two Fab fragments. In another nonlimiting example, the enzyme pepsin is used to cleave the antibody into a constant-region fragment (Fc) and a fragment comprising both antigen-binding domains F(ab′)₂. In certain embodiments, the Fab fragments are recombinant and produced without the need for enzymatic digestion of full-length antibodies. It is also contemplated that the antigen-binding domain can comprise a complex of one or more individual antigen-biding domains bound together. Examples of such antigen-binding complexes include, but are not limited to minibodies, diabodies, triabodies, tetrabodies, among others. The skilled artisan would be able to select a particular antigen-binding domain for the bispecific antibody or BiTE of the invention based on the nature of the desired target cell and T cell antigens.

As used herein, the term “single-chain variable fragment” or “scFv” is a fusion protein of the variable regions of the heavy (VH) and light chains (VL) of an immunoglobulin (e.g., mouse or human) covalently linked to form a VH::VL heterodimer. The heavy (VH) and light chains (VL) are either joined directly or joined by a peptide-encoding linker or spacer, which connects the N-terminus of the VH with the C-terminus of the VL, or the C-terminus of the VH with the N-terminus of the VL.

As used herein. “Fab” refers to a fragment of an antibody structure that binds to an antigen but is monovalent and does not have a Fc portion, for example, an antibody digested by the enzyme papain yields two Fab fragments and an Fc fragment (e.g., a heavy (H) chain constant region; Fc region that does not bind to an antigen).

As used herein. “F(ab′)₂” refers to an antibody fragment generated by pepsin digestion of whole IgG antibodies, wherein this fragment has two antigen binding (ab′) (bivalent) regions, wherein each (ab′) region comprises two separate amino acid chains, a part of a H chain and a light (L) chain linked by an S—S bond for binding an antigen and where the remaining H chain portions are linked together. A “F(ab′)₂” fragment can be split into two individual Fab′ fragments.

### Methods

The BiTEs of the invention may be included in a composition suitable for use as an immunotherapy. The composition may include a pharmaceutical composition and further include a pharmaceutically acceptable carrier. A therapeutically effective amount of the pharmaceutical composition comprising the BiTEs may be administered.

Also included is a method of treating a disease or condition in a subject in need thereof comprising administering to the subject a BiTE (e.g., a switch-BiTE). In one embodiment, the method of treating a disease or condition in a subject in need thereof comprises administering to the subject a switch-BiTE.

Also included is a method of treating a cancer in a subject in need thereof comprising administering to the subject a switch-BiTE of the current invention, and then administering to the subject an effective amount of the small molecule disrupting agent to deactivate the switch-BiTE in response to at least one off-tumor effect caused by the switch-BiTE. In certain embodiments, the disrupting agent is amantadine.

In another aspect, the invention includes methods of generating an immune response in a subject, comprising administering to the subject an effective amount of the switch-BiTE of the invention sufficient to prime an immune response against the target cell, followed by an effective amount of the small molecule disrupting agent sufficient to disrupt and deactivate the switch-BiTE. In this way, antigens and pro-inflammatory molecules released from the target cells prime endogenous T cell and antibody responses against the target cells that continue after the deactivating of the switch-BiTEs. In certain embodiments, the switch-BiTEs can be administered more than once. In certain embodiments, the immune-priming switch-BiTE treatment can be combined with another switch-BiTE in order to augment the anti-target cell immune response. In both examples, the disruptive agent can be administered at a later time after sufficient clearance of the disease causing cells or in response to toxic off-tumor effects of the switch-BiTEs.

The administration of the BiTEs of the invention may be carried out in any convenient manner known to those of skill in the art. The BiTEs of the present invention may be administered to a subject by aerosol inhalation, injection, ingestion, transfusion, implantation or transplantation. The compositions described herein may be administered to a patient transarterially, subcutaneously, intradermally, intratumorally, intranodally, intramedullary, intramuscularly, by intravenous (i.v.) injection, or intraperitoneally. In other instances, the cells of the invention are injected directly into a site of inflammation in the subject, a local disease site in the subject, a lymph node, an organ, a tumor, and the like.

In some embodiments, a dose of BiTEs as well as the disruptive agents are administered to a subject in need thereof, in a single dose or multiple doses. The BiTEs and disruptive agents of the invention can be administered in dosages and routes and at times to be determined in appropriate pre-clinical and clinical experimentation and trials. Compositions comprising BiTEs and the disruptive agent may be administered multiple times at dosages within these ranges. Administration of the BiTEs of the invention as well as the disruptive agent used to deactivate them may be combined with other methods useful to treat the desired disease or condition as determined by those of skill in the art.

### Pharmaceutical Compositions and Formulations

Also provided are compositions including the cells for administration, including pharmaceutical compositions and formulations, such as unit dose form compositions including the amount of BiTEs and disruptive agent for administration in a given dose or fraction thereof. The pharmaceutical compositions and formulations generally include one or more optional pharmaceutically acceptable carrier or excipient.

The term “pharmaceutical formulation” refers to a preparation which is in such form as to permit the biological activity of an active ingredient contained therein to be effective, and which contains no additional components which are unacceptably toxic to a subject to which the formulation would be administered. A “pharmaceutically acceptable carrier” refers to an ingredient in a pharmaceutical formulation, other than an active ingredient, which is nontoxic to a subject. A pharmaceutically acceptable carrier includes, but is not limited to, a buffer, excipient, stabilizer, or preservative. In some aspects, the choice of carrier is determined in part by the particular BiTE and/or by the method of administration. Accordingly, there are a variety of suitable formulations. For example, the pharmaceutical composition can contain preservatives. Suitable preservatives may include, for example, methylparaben, propylparaben, sodium benzoate, and benzalkonium chloride. In some aspects, a mixture of two or more preservatives is used. The preservative or mixtures thereof are typically present in an amount of about 0.0001% to about 2% by weight of the total composition. Carriers are described. e.g., by Remington's Pharmaceutical Sciences 16th edition, Osol, A. Ed. (1980). Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins: hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA: sugars such as sucrose, mannitol, trehalose or sorbitol; salt-forming counter-ions such as sodium; metal complexes (e.g. Zn-protein complexes); and/or non-ionic surfactants such as polyethylene glycol (PEG).

Buffering agents in some aspects are included in the compositions. Suitable buffering agents include, for example, citric acid, sodium citrate, phosphoric acid, potassium phosphate, and various other acids and salts. In some aspects, a mixture of two or more buffering agents is used. The buffering agent or mixtures thereof are typically present in an amount of about 0.0010% to about 4% by weight of the total composition. Methods for preparing administrable pharmaceutical compositions are known. Exemplary methods are described in more detail in, for example, Remington: The Science and Practice of Pharmacy, Lippincott Williams & Wilkins; 21st ed. (May 1, 2005).

The formulations can include aqueous solutions. The formulation or composition may also contain more than one active ingredient useful for the particular indication, disease, or condition being treated with the cells, preferably those with activities complementary to the cells, where the respective activities do not adversely affect one another. Such active ingredients are suitably present in combination in amounts that are effective for the purpose intended. Thus, in some embodiments, the pharmaceutical composition further includes other pharmaceutically active agents or drugs, such as chemotherapeutic agents. The pharmaceutical composition in some embodiments contains the bispecific antibodies and BiTEs in amounts effective to treat or prevent the disease or condition, such as a therapeutically effective or prophylactically effective amount. Therapeutic or prophylactic efficacy in some embodiments is monitored by periodic assessment of treated subjects. The desired dosage can be delivered by a single bolus administration, by multiple bolus administrations, or by continuous infusion administration of the bispecific antibodies and BiTEs or disruptive agent.

The formulations to be used for in vivo administration are generally sterile. Sterility may be readily accomplished, e.g., by filtration through sterile filtration membranes.

The contents of the articles, patents, and patent applications, and all other documents and electronically available information mentioned or cited herein, are hereby incorporated by reference in their entirety to the same extent as if each individual publication was specifically and individually indicated to be incorporated by reference. Applicants reserve the right to physically incorporate into this application any and all materials and information from any such articles, patents, patent applications, or other physical and electronic documents.

While the present invention has been described with reference to the specific embodiments thereof, it should be understood by those skilled in the art that various changes may be made and equivalents may be substituted without departing from the true spirit and scope of the invention. It will be readily apparent to those skilled in the art that other suitable modifications and adaptations of the methods described herein may be made using suitable equivalents without departing from the scope of the embodiments disclosed herein. In addition, many modifications may be made to adapt a particular situation, material, composition of matter, process, process step or steps, to the objective, spirit and scope of the present invention. All such modifications are intended to be within the scope of the claims appended hereto. Having now described certain embodiments in detail, the same will be more clearly understood by reference to the following examples, which are included for purposes of illustration only and are not intended to be limiting.

### EXAMPLES

Various embodiments of the present application can be better understood by reference to the following Examples which are offered by way of illustration. The scope of the present application is not limited to the Examples given herein.

### Materials and Methods

### Materials

Chemicals and antibodies: Anti-human HER2 (Catalog: BE0277, clone: 7.16.4), anti-mouse CD3 (Catalog: BE001-1FAB, clone: 145-2C11 f(ab′)2 Fragments) and anti-human CD3 (Catalog: BE0231, clone: UCHTI (Leu-4) (T3)) antibodies were purchased from BioXcell. Flow antibodies anti-mouseCD45-Brilliant Violet 421 (Catalog: 103133, clone: 30-F11, 1:100 dilution), anti-mouseCD3-PE (Catalog: 100206, clone: 17A2.1:100 dilution), anti-mouseCD4-Alexa Fluor® 488 (Catalog: 100423, clone: GK1.5, 1:100 dilution), anti-mouseCD8-APC (Catalog: 100712, clone: 53-6.7, 1:100 dilution), anti-mouseFoxP3-Brilliant Violet 421 (Catalog: 126419, clone: MF-14, 1:100 dilution), anti-mouseCD25-Brilliant Violet 711 (Catalog: 102049, clone: PC61, 1:100 dilution), anti-mouseCD44-Brilliant Violet 421 (Catalog: 103039, clone: IM7, 1:100 dilution), anti-mouseCD62L-Brilliant Violet 711 (Catalog: 104445, clone: MEL-14, 1:100 dilution) were purchased from Biolegend. NH₂-PEG2000-MAL was obtained from Creative PEGWorks. β-CDs, and 3-phenylpropanoyl chloride were obtained from Sigma. Mouse IgG1 Fab kit was order from New England Biolabs (Catalog: P0770S), Mouse IgG2a Fab purification kit was ordered from Thermo Fisher (Catalog: 44985). The piggyBac transposase vector pCMV-hyPBase and the pPB7 TBG.human HER2.P2A.IRFP720 plasmids that were used to construct the HER2 expression model were provided. All of the cell lines were tested negative for mycoplasma.

### Animals

C57BL/6 mice (female, 6-8 weeks) were ordered from Jackson laboratory and housed in a specific-pathogen-free animal facility at ambient temperature (22±2° C.), air humidity 40%-70% and 12-h dark/12-h light cycle. NSG-SGM3 mice (female. 6-8 weeks) were ordered from Jackson laboratory. For IVIS imaging, an alfalfa- and fenbendazole-free mouse diet was used to decrease the background fluorescence signal.

### Antibody Fab Fragment Preparation

The Fab fragments of anti-mouse CD3 and anti-human HER2 antibodies were prepared using commercialized Fab preparation kits following manufacturers' manuals. Before conjugation of phenyl-PEG-MAL or β-CD-PEG-MAL to the antibody fragments, antibodies were treated with 2-iminothiolane to enhance the number of thiol groups on the antibodies.

Modifying CD3 and HER2 Fabs with 2-iminothiolane

There are only two thiol groups on each Fab fragment. The number of thiol groups on the fragments were increased using a reported method. To prepare the thiol-modified antibody fragments, 2-iminothiolane was added to 500 μg of antibody fragment and the mixture was incubated at 37° C. for 4 h. Free 2-iminothiolane was removed using a centrifugal filter (molecular weight cut off: 3 kDa). The free thiol groups on the antibody Fabs before and after 2-iminothiolane modification were measured using a commercialized free thiol assay kit (ab112158). Since it was known that each unmodified Fab contains 2 free thiol groups after mild reduction, the total thiols on each antibody fragment (N) was calculated using the following equation:

N=[(total thiol groups after 2-iminothiolane modification)/(total thiol groups before 2-iminothiolane modification)]×2.

It was found that on average 4.7 and 4.9 thiol groups were added to the CD3 Fab and HER2 Fab, respectively.

### Preparation of the Switchable Bispecific T Cell Nanoengagers

First, PEG-phenyl and PEG-β-CD conjugations were prepared. For CD3 Fab-PEG-phenyl1 synthesis, the anti-CD3 Fab was mixed with MAL-PEG-phenyl at a 1:1 ratio in PBS for 2 h with gentle stirring at room temperature, then unconjugated MAL-PEG-phenyl or MAL-PEG-β-CD were removed using a centrifugal filter with a molecular weight cut off of 10 KDa. After washing with PBS several times, the antibody Fab-PEG-phenyl or Fab-PEG-β-CD was stored at 4° C. for future applications. CD3 Fab-PEG-phenyl2, CD3 Fab-PEG-phenyl3 and CD3 Fab-PEG-phenyl4 were synthesized using the same protocol except a 1:2, 1:3, or 1:4 ratio of CD3 Fab to MAL-PEG-phenyl was used, respectively. HER2 Fab-PEG-β-CD1. HER2 Fab-PEG-β-CD2, and HER2 Fab-PEG-3-CD3 were synthesized with similar steps except a 1:1, 1:2, 1:3, or 1:4 ratio of HER2 Fab and MAL-PEG-β-CD was used, respectively.

### Quantification of the Composition of SiTEs

The compositions of SiTEs were determined using a two-step separation and quantification method. To quantify the composition of each SiTE, 1 mg of different SiTEs were firstly loaded on a centrifugal filter device with molecular weight cut off (MWCO) of 50 kDa to separate all free Fabs. To ensure the complete separation of free Fabs, SiTEs were washed two times with 1×PBS. The three elutes (contain free Fab) were combined and stored for quantification. The Fab clusters that cannot pass through the 50 kDa filter were collected and loaded on a centrifugal filter device with MWCO of 100 kDa. With similar steps, Fab dimers (MW 50-100 kDa) were collected. Fab clusters that cannot pass through the 100 kDa filter were collected and loaded on a centrifugal filter device with MWCO of 300 kDa and Fab clusters with MW from 100-300 kDa were collected. The Fab clusters that cannot pass through the 300 kDa filter were collected and loaded on a centrifugal filter device with MWCO of 1000 kDa. The Fab clusters with molecular weights in the range of 300-1000 kDa were collected. The Fab clusters that cannot pass through the 1000 kDa were also collected. It was found that these are mostly nanoparticles when observed under TEM. The various components were quantified using bicinchoninic acid (BCA) assay and their relative percentage was calculated.

### Stability of SiTEs in the Present of AMD, Phenylalanine, or Tyrosine

10 μg of SiTEs 1-4 were incubated with 500 μg AMD, 500 μg phenylalanine, or 500 μg tyrosine at 37° C. for 2 h, then the components in sample was analyzed using the two-step separation and quantification method as mentioned above.

### Size-Exclusion Chromatography and Circular Dichroism

SEC experiments were performed using 100 μL injections with a Superose 6 Increase 10/300 column at 0.5 ml/min at 25° C. The mobile phase consisted of 20 mM TrisHCl buffer (pH 7.4) containing 150 mM NaCl, filtered with 0.22 μm nylon membrane, and degassed.

Circular dichroism experiments were performed on an Aviv 202 Circular Dichroism system. Antibody samples were dissolved in pure water and loaded in 1 cm quartz cells at room temperature. The spectra were recorded over a wavelength range 200-240 nm at sample concentration of 0.2 mg/mL at 20° C. The final spectra were the average of 20 scans. CD spectra of the buffer solutions in the appropriate cuvette were subtracted from the sample spectra as background.

### Fluorescence Resonance Energy Transfer Experiment

CD3 Fab-PEG-phenyl and HER2 Fab-PEG-β-CD were labelled with Cell tracer 450 and flourescein isothiocyanate (FITC), respectively. For a typical synthesis, 100 μg of CD3 Fab-PEG-phenyl was mixed with 5 nmol of Cell tracer 450 in PBS buffer at room temperature under gentle stirring. After 2 h, the mixture was collected and dialyzed against PBS for 48 h. FITC labeled HER2 Fab-PEG-β-CD was prepared with similar steps except FITC was used. The photoluminescence spectrum of the Cell tracer 450-labelled CD3 Fab-PEG-phenyl, FITC-labeled HER2 Fab-β-CD, and the mixture of these two molecules were measured on a HORIBA FluoroMax-3 fluorescence spectrometer with excitation wavelength of 405 nm, and emission spectrum were collected from 420 nm-700 nm.

### Gel Electrophoresis and DLS

Protein samples were loaded on the polyacrylamide basic native gel (4-12%). Running conditions: 120V, 45 min. MES-SDS buffer was used. For DLS, SiTEs with concentrations of 50 g/mL were used and the data was collected on a Malvern Zetasizer Nano machine.

### Generation of Primary Mouse T Cells

Primary mouse T cells were isolated from the spleen of 6-week-old female C57BL/6 mice. After mice were euthanized, the spleens were collected, cut into pieces, and homogenized on a 100 μm cell strainer. Then red blood cells were lysed with ACK lysing buffer, and the cell suspensions were passed through a 70 μm cell strainer. CD3⁺ T cells were collected using a STEMCELL T cell isolation kit resulting in approximately 2×10⁷ CD3⁺ T cells obtained from each mouse spleen.

### Ex Vivo Killing Assays

The target cells, E0771-HER2 or E0771 that express a luciferase reporter were incubated with primary mouse CD3⁺ T cells with different treatments. After 24 h, target cell viability was measured by determining the luciferase expression level using a luciferase assay kit. In additional assays, Raji cells were tagged with both GFP and luciferase, and the viability of Raji cells was monitored using a luciferase assay kit.

### Quantification of Cell-to-Cell Interactions

E0771-HER2 cells were labeled with CSFE-green and primary T cells were labelled with CFSE-red. The two cells were co-cultured and then treated with PBS or SiTE for 2 h. After that, the cells were observed by confocal imaging. T cell to T cell interactions and T cell to tumour cell interactions were counted manually by taking 10 images for quantification.

### Determination of Cytokine Concentrations and Liver Enzymes

Mouse blood was collected at different time points and the serum was obtained by centrifuging the clotted blood. Cell culture medium was collected at different time points. For the detection of various cytokines in cell culture medium or mouse serum, 100 μL of medium or serum was used for each sample and the cytokines (IL-6, IL-1, IFN-γ, TNF-α, CXCL10, or CCLS) were detected using either ELISA kits or a FirePlex®-96 Key Cytokines Immunoassay Panel (catalog. No: ab243549) following manufacturers protocol. Mouse serum was collected and the alanine aminotransferase (ALT) and aspartate aminotransferase (AST) levels were detected using an alanine transaminase colorimetric activity assay kit (Cayman, catalog. No: 700260) or an aspartate aminotransferase colorimetric activity assay Kit (Cayman, catalog. No: 701640).

### Quartz Crystal Microbalance

Gold-coated QCM-D sensors with a resonance frequency of 4.95 MHz were first cleaned using a plasma cleaner and then were rinsed with PBS. A Q-Sense E4 QCM-D system was used to monitor the shifts in the resonance frequency (AF) and dissipation (AD) for odd overtones (n=1, 3, 5, 7, 9, 11, and 13). CD3 or HER2 protein was adsorbed on the gold-coated sensor by flowing 0.2 mg/mL CD3 or HER2 in PBS over the crystals. After ~10 min of adsorption, the CD3 or HER2 layers were rinsed with PBS until equilibration was achieved (~15 min). Interactions between free CD3 Fab or SiTE and CD3 protein were measured by flowing 200 g/mL CD3 Fab or SiTE in PBS over the adsorbed CD3 protein layer. Interactions between free HER2 Fab or SiTE and HER2 protein were tested by flowing 200 g/mL HER2 Fab or SiTE in PBS over the adsorbed HER2 protein layer. PBS buffer was introduced using continuous pumping after about 50 min of adsorption of the nanoparticles onto the antigen layer. Control measurements were performed by flowing free CD3 or HER2 Fab solutions over clean gold-coated sensors until the frequency change had plateaued, then CD3 or HER2 Fab-adsorbed surface was rinsed with PBS to remove any free antibody fragments.

### Binding-Affinity Assay

Binding affinity of antibodies to their target proteins were determined using a reported method. The HER2-overexpressing E0771-HER2 cells was cultured in 96-well cell culture plates and grown to approximately 80% confluence. The cells were then fixed with 4% formaldehyde and incubated with various concentrations of SiTE (labeled with FITC) and blocked with bovine serum albumin (BSA) for 30 min. Unbound SiTE were then removed with three washes with PBS. Absorbance was measured at 488 nm, with non-fluorescently labeled SiTE used as a control. The dissociation constant Kd was obtained by plotting normalized absorbance values versus concentrations of the SiTE added to the cell cultures. To measure the Kd for CD3 Fab binding to CD3 antigen and SiTE binding to CD3 antigen, 96-well plates were first coated using retronectin to make the T cells adhere to the plates. Then the Kd of free CD3 Fab to CD3 antigen and SiTE to CD3 antigen were determined using similar experiments described above.

### Flow Experiments

Mice were euthanized with CO₂ and perfused with a 37° C. PBS buffer containing collagenase IV (0.5 mg/mL), dispase (50 units/mL) and DNase (50 units/mL). After 30 min of digestion, the tumour tissue was cut into small pieces and homogenized on a 100 μm cell strainer. Then red blood cells were lysed with ACK lysing buffer and the cell suspensions were passed through a 70 μm cell strainer. 1 million cells were stained with different antibodies for 30 min and washed two times with PBS before analysis on the flow cytometer (LSR, BD).

### Construction of the Humanized Immune System Mice Model

Recently, a study developed a humanized immune system NSG-SGM3 mouse model and found that when CAR T cells were infused to kill cancer cells it can induce several major symptoms of human CRS and neurotoxicity. Here, a humanized mouse model was constructed following procedures known to those of ordinary skill in the art with some modification. NSG-SGM3 mice were treated with busulfan (40 mg/kg) to remove the bone marrow, then 10⁵ human fetal liver CD34⁺ cells were i.v. injected to the mice. The development of human immune cells was confirmed after 5 weeks post-CD34 fetal liver cell infusion.

Construction of the Mice Model with Human HER2 or CD3 Expression in the Liver

A mouse model has been recently reported with stable expression of the human HER2 antigen in the liver. A hydrodynamic injection method was used to deliver a piggyBac transposase vector pCMV-hyPBase and the pPB7 TBG.human HER2.P2A.IRFP720 plasmids to the mouse liver. Here, the animal model construction was modified and delivered the two plasmids with a MC-3 lipid nanoparticle (LNP) ENREF_46. The LNPs were synthesized using a microfluidic device to mix an aqueous phase containing plasmids and an ethanol phase containing DLIN-MC3-DMA cationic lipid, 1,2-distearoyl-sn-glycero-3-phosphocholine (DSPC), C14-PEG2000, and cholesterol. The LNP was dialyzed against PBS and passed through a 0.22 μm filter before use. LNPs were i.v. injected to mice at a dose of 2 μg plasmid/mouse. Human CD3 antigen was expressed in the liver of NSG-SGM3 mice using LNPs delivering pPB-CMV-hCD19:T2A:EGFP and pCMV-hyPBase plasmids.

### Statistics

Graphpad prism 7.0 software was used to conduct statistical analysis. Error bars represent means±standard deviation (s.d.). Animal experiments were conducted after randomization, and 7-8 mice were used in each group. A two-tailed Student's t-test was used to calculate the statistical differences between two groups. The differences in animal survival experiments were calculated using the Kaplan-Meier method, and the log-rank test was used to determine the P values.

### Example 1: Synthesis and Characterization of the Switchable Bispecific T Cell Nanoengager

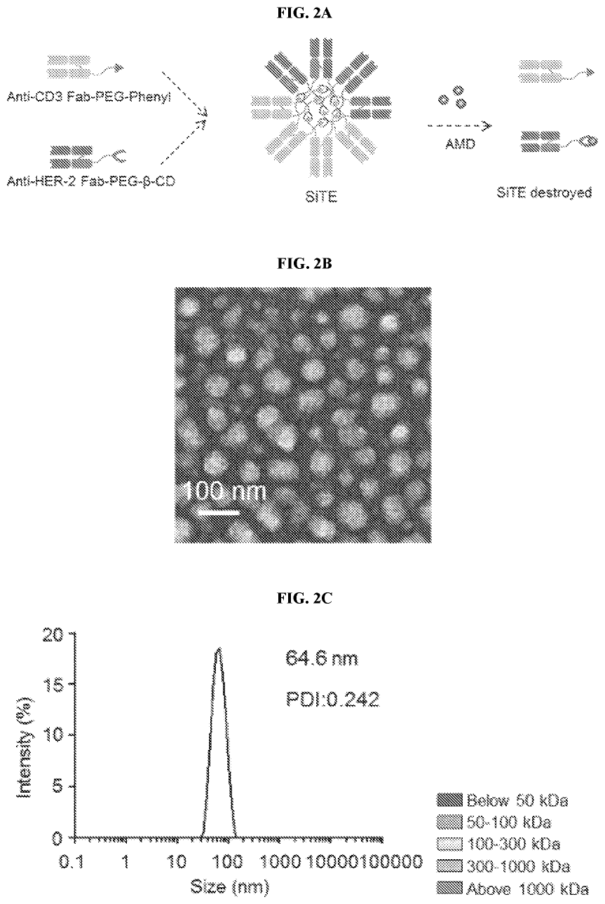

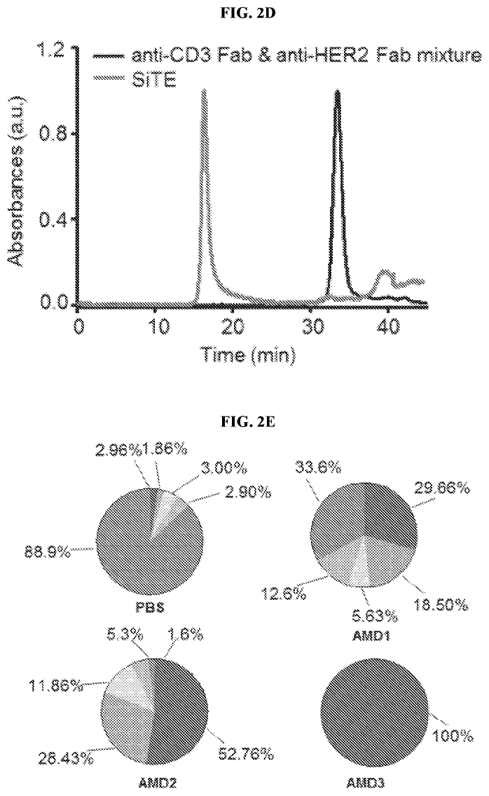

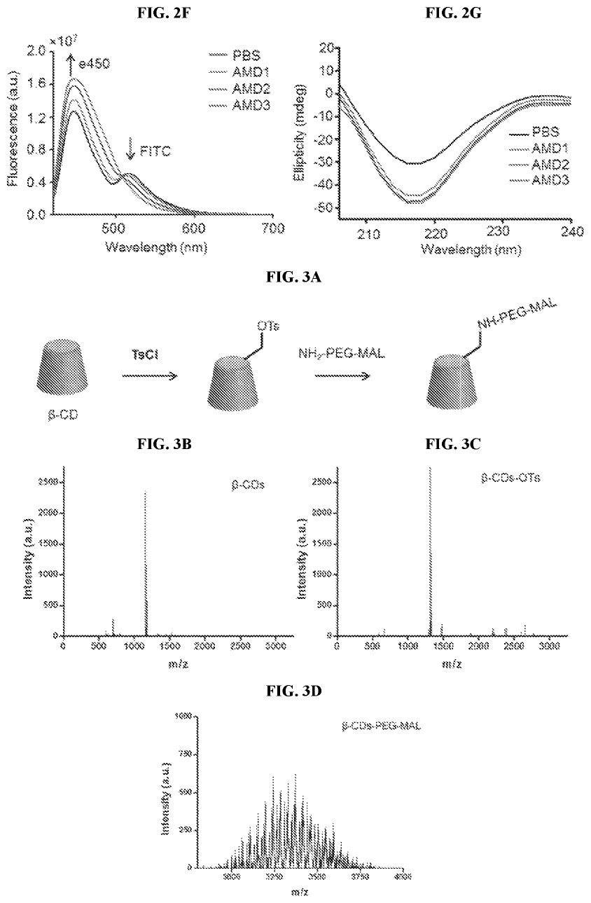

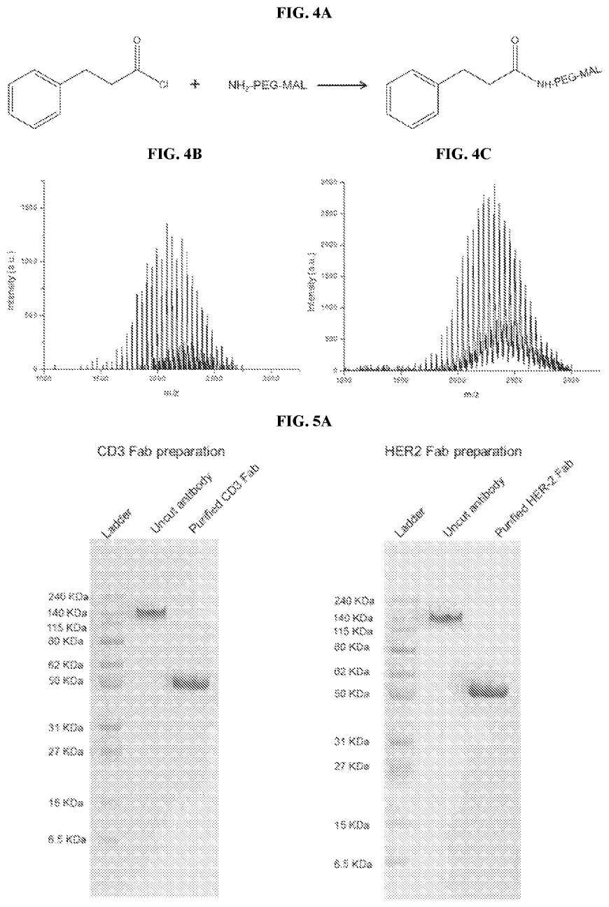

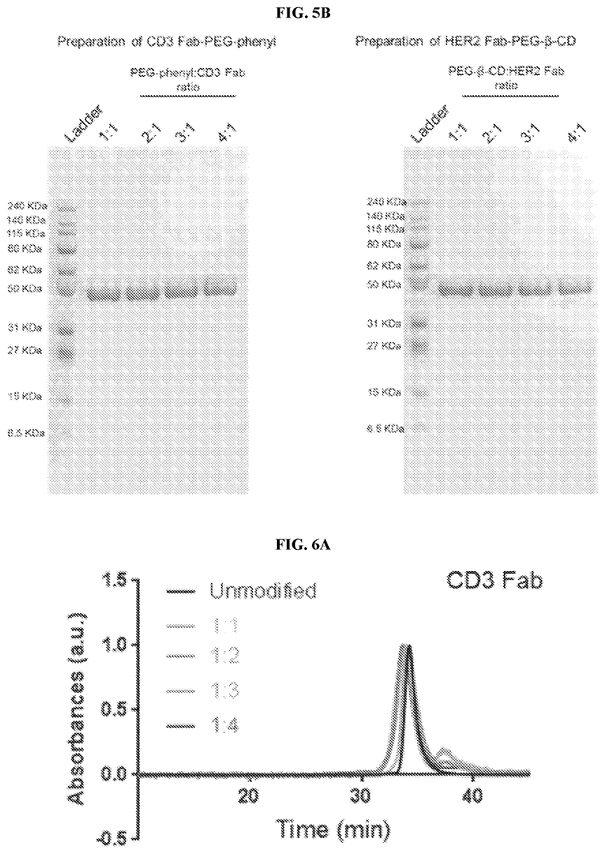

In this study, SiTE was designed to bind both CD3 for T cell engagement and HER2 for tumour targeting and break down when exposed to AMD (FIG. 2A). To begin SiTE synthesis, phenyl-modified poly ethyleneglycol (PEG-phenyl) and β-cyclodextrin-modified PEG (PEG-β-CD) were synthesized and then were characterized by using matrix-assisted laser desorption/ionization time of flight mass spectrometry (MALDI-TOF-MS, FIGS. 3A-3D and FIGS. 4A-4C). Fab fragments of antibodies for CD3 and HER2 were prepared using papain protease or IdeZ protease, respectively, and gel electrophoresis was used to confirm preparation of these Fab fragments (FIG. 5A). The CD3 and HER2 Fab fragments were then treated with 2-iminothiolane to increase the number of thiol groups on their surface. The number of thiol groups were increased to 4.7 and 4.9 for each CD3 and HER2 Fab, respectively. Then CD3 and HER2 Fabs were conjugated to PEG-phenyl and PEG-β-CD, respectively (random rather than site-specific conjugation was used for all experiments described herein). These reactions to form CD3 Fab-PEG-phenyl and HER2 Fab-PEG-β-CD were performed at Fab to polymer (PEG conjugate) molar ratios of 1:1, 1:2, 1:3, and 1:4, and the products were characterized via gel electrophoresis (FIG. 5B), revealing that the conjugation of PEG-phenyl and PEG-β-CD to the Fab fragments did not induce aggregation at any of these ratios. Moreover, size exclusion chromatography (SEC, FIGS. 6A-6B) was used to quantify the number of PEG-phenyl and PEG-3-CD conjugated to each CD3 and HER2 Fabs. The results indicated that approximately 1.12, 2.36, 3.40, and 4.84 MAL-PEG-phenyls were conjugated to each CD3 Fab when reacted at CD3 to PEG-phenyl ratios of 1:1, 1:2, 1:3, and 1:4, respectively. On average 1.15, 2.39, 4.09, and 4.91 MAL-PEG-beta-CDs were conjugated to each HER2 Fab when reacted at HER2 Fab to MAL-PEG-beta-CD ratios of 1:1, 1:2, 1:3, and 1:4, respectively.

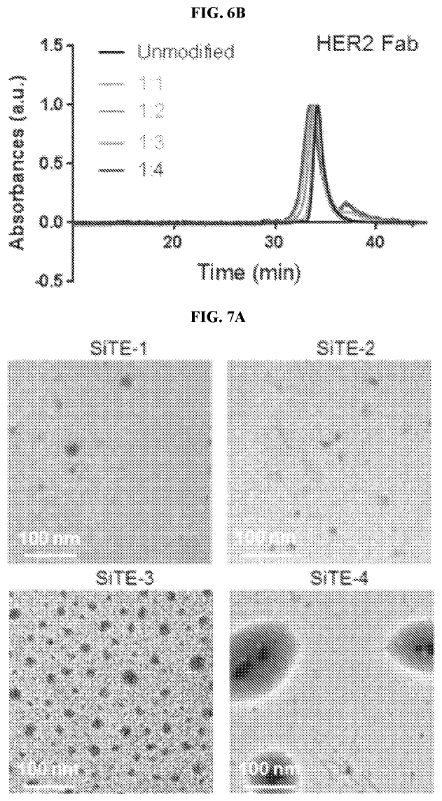

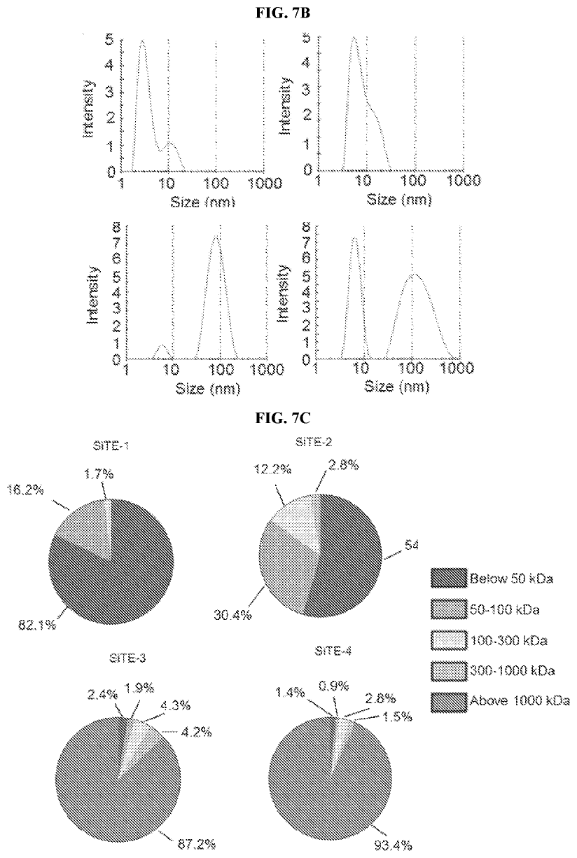

Next, the SiTE was assembled by mixing CD3 Fab-PEG-phenyl with HER2 Fab-PEG-β-CD in phosphate buffered solution (PBS). Specifically, SiTE-1 was made using CD3 Fab-PEG-phenyl and HER2 Fab-PEG-β-CD that were both synthesized at the 1:1 Fab:PEG conjugate ratio while SiTE-2, SiTE-3, and SiTE-4 used those made at 1:2, 1:3, and 1:4 Fab:PEG conjugate ratios, respectively. The phenyl groups from CD3 Fab-PEG-phenyl interact with the β-CD in HER2 Fab-PEG-β-CD mediated the assembly of the two antibodies. The interactions of phenyl groups in phenylalanine, tyrosine, and tryptophan in CD3 antibody with β-CD in HER2 Fab-PEG-β-CD may also contributed to the assembly of the SiTEs. FIGS. 7A-7B show transmission electron microscope (TEM) images and dynamic light scattering (DLS) size distribution data for SiTE1-4. The compositions of the four SiTEs were determined using a two-step separation and quantification method. The percentages of different components are shown in FIG. 7C.

It was next tested if the addition of AMD, which shows much higher affinity to β-CD compared to phenyl, can be used to separate the CD3 Fab-PEG-phenyl from the HER2 Fab-PEG-β-CD and ultimately “switch off” the SiTEs. 10 μg of SiTEs were mixed with excess (500 μg) AMD and found (Tables 1a-1c) that AMD induced complete disassembly of various SiTEs, demonstrating that AMD could be used to control SiTE structure and thus, potentially their activity (Tables 1a-1c).

TABLE 1a
Treatment of SiTEs with AMD

| MW Range (kDa) | SiTE-1 | SiTE-2 | SiTE-3 | SiTE-4 |
|---|---|---|---|---|
| <50 | 100 | 100 | 100 | 100 |
| 50-100 | 0 | 0 | 0 | 0 |
| 100-300 | 0 | 0 | 0 | 0 |
| 300-1000 | 0 | 0 | 0 | 0 |
| Above 1000 | 0 | 0 | 0 | 0 |

TABLE 1b
Treatment of SiTEs with phenylalanine

| MW Range (kDa) | SiTE-1 | SiTE-2 | SiTE-3 | SiTE-4 |
|---|---|---|---|---|
| <50 | 95.2 | 97.4 | 6.3 | 5.2 |
| 50-100 | 4.8 | 2.6 | 2.9 | 2.3 |
| 100-300 | 0 | 0 | 2.2 | 0.3 |
| 300-1000 | 0 | 0 | 7.1 | 3.6 |
| Above 1000 | 0 | 0 | 81.5 | 88.6 |

TABLE 1c
Treatment of SiTEs with tyrosine

| MW Range (kDa) | SiTE-1 | SiTE-2 | SiTE-3 | SiTE-4 |
|---|---|---|---|---|
| <50 | 97.5 | 98.4 | 7.4 | 4.5 |
| 50-100 | 2.5 | 1.6 | 3.7 | 3.7 |
| 100-300 | 0 | 0 | 1.8 | 1.8 |
| 300-1000 | 0 | 0 | 7.3 | 3.6 |
| Above 1000 | 0 | 0 | 79.8 | 86.4 |

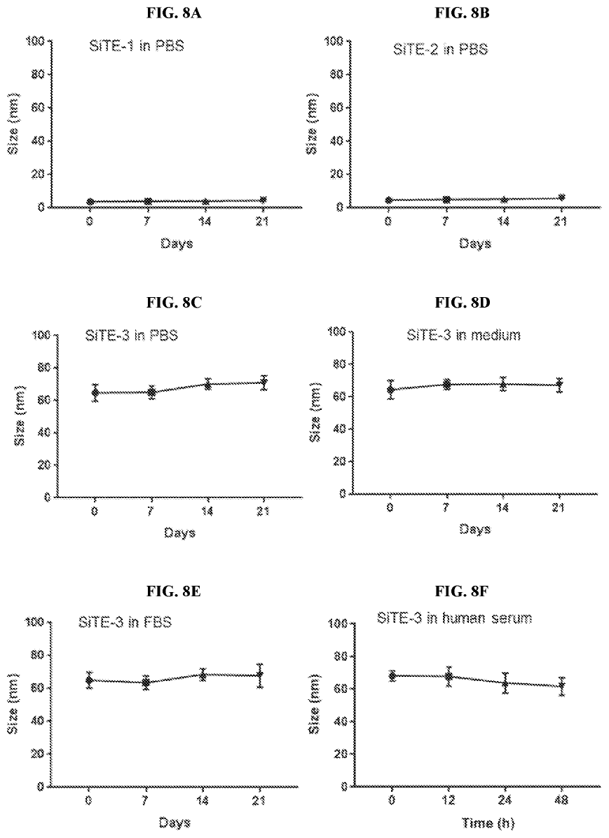

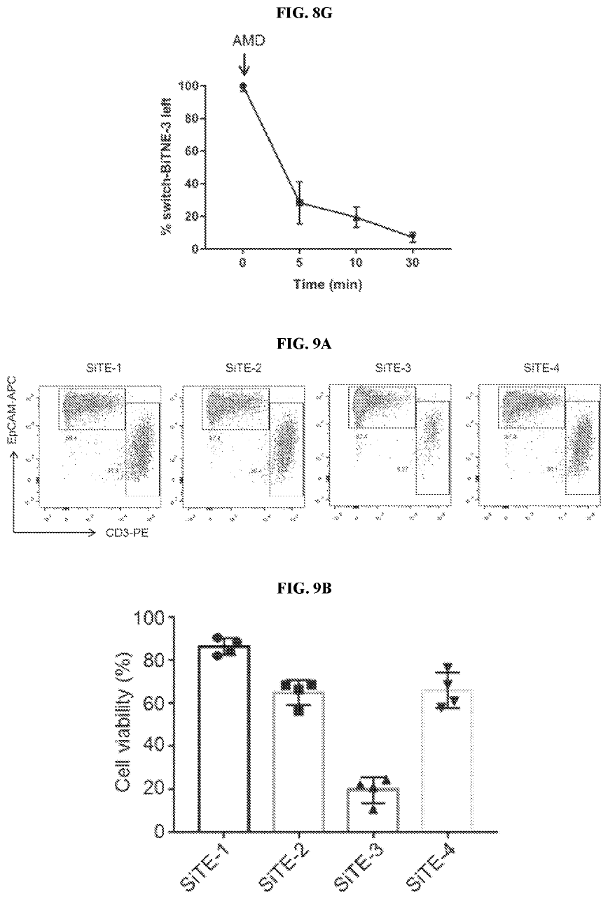

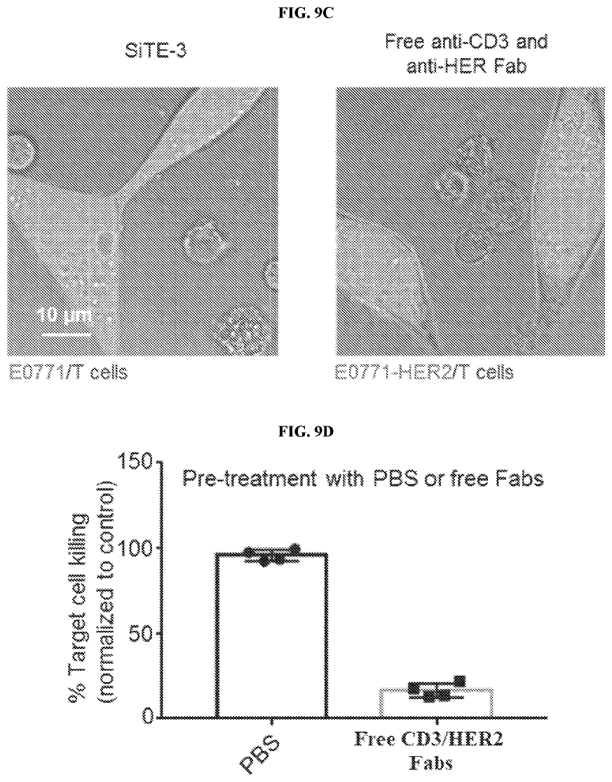

Two additional small molecules (i.e., phenylalanine and tyrosine) were tested for their ability to separate these antibody fragments (Tables 1a-1c). It was found that both phenylalanine and tyrosine treatment induced the separation of the CD3 Fab from the HER2 Fab in SiTE-1 and 2. However, neither phenylalanine nor tyrosine induced much separation in SiTE-3 and 4 (Tables 1b-1c). This is likely because SiTEs 1 and 2 consist mainly of the Fab dimer, which is formed via a single supramolecular interaction between the phenyl group and β-CD. However. SiTE-3 and 4 contain mostly Fab nanoparticles that form via more stable multivalent host-guest interactions between the phenyl group and β-CD. Next, to ensure the stability of SiTEs in the absence of AMD, SiTEs were stored at 4° C. in PBS, complete medium, and mouse serum and were then characterized for size over the course of 3 weeks. Over the course of this study, no changes in size were observed, suggesting that the SiTEs were mostly stable over this time period (FIGS. 8A-8G). Finally, to compare their tumour killing efficacy, each SiTE was incubated with a mix of HER2⁺ E0771 breast cancer cells and primary mouse T cells for 24 h (FIGS. 9A-9B). The results showed HER2-specific engagement from the SiTEs and tumour cell toxicity, with SiTE-3 demonstrating the greatest tumour cell lysis (FIG. 9B). Thus, SiTE-3 was selected for further exploration and is referred to as the “SiTE” herein in subsequent experiments for brevity.

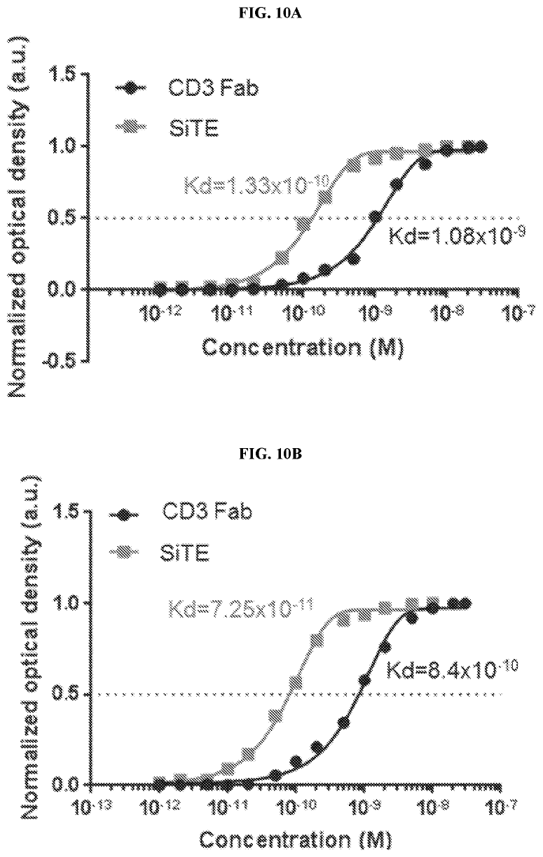

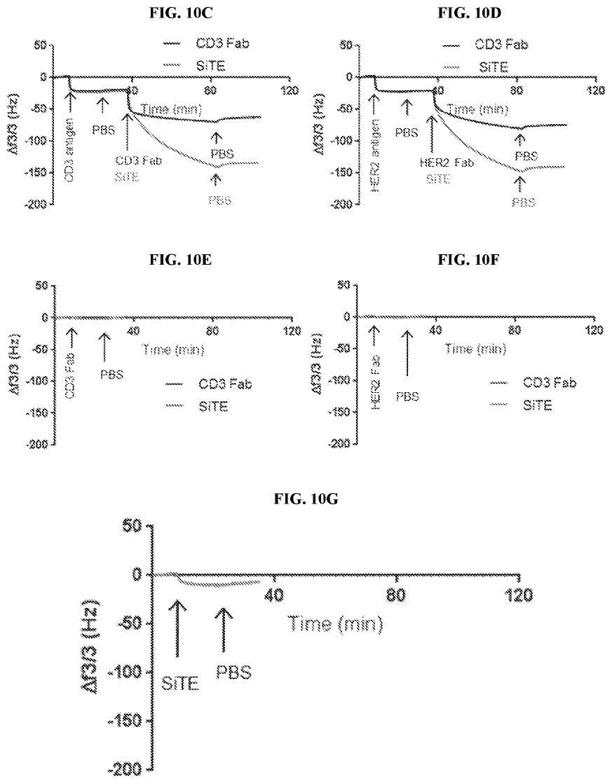

The SiTE was first characterized by using TEM imaging, DLS, and SEC (FIGS. 2B-2D). Additionally, the affinity of the CD3 Fab and SiTE to CD3 antigen and the affinity of the HER2 Fab and SiTE to HER2 antigen were determined using a reported method. As shown in FIGS. 10A-10B, the dissociation constant (Kd) values of free CD3 Fab against CD3 protein and free HER2 Fab to HER2 protein were 1.08×10⁻⁹ M and 8.4×10⁻¹⁰ M, respectively. Surprisingly, the Kd value of the SiTE to CD3 antigen and HER2 antigen decreased to 1.33×10⁻¹⁰ M and 7.25×10⁻¹¹ M, respectively. These results demonstrated that the conjugation and assembly of the CD3 and HER2 Fabs increased the avidity of both CD3 and HER2 Fabs. This may be a result of the multivalent effect of the SiTE. The binding of CD3 Fab, HER2 Fab, and SiTE to their target antigens were further evaluated using quartz crystal microbalance (QCM, FIGS. 10C-10G). SiTE showed improved binding to the CD3- and HER2-protein coated quartz crystal compared to free CD3 and HER2 Fabs (FIGS. 10C-10G). These results further demonstrate the higher binding affinity of the SiTE to both CD3 and HER2 proteins. The TEM and DLS confirmed a homogeneous SiTE solution with diameter of approximately 66 nm. Further, SEC results (FIG. 2D) showed a main peak at around 16.3 min for SiTE, indicating an average composition of >20 Fab monomers per SiTE. A two-step separation and quantification method was next used to determine if SiTE can be disassembled upon the addition of AMD (FIG. 2E). The results showed that AMD can induce the disassembly of SiTE in a dose-dependent manner. To further confirm this AMD-induced SiTE disassembly, fluorescence resonance energy transfer (FRET) was also used. Specifically, the CD3 Fab was modified with Cell tracer 450 (e450) and the HER2 Fab was modified with fluorescein isothiocyanate (FITC). Thus, minimal e450 signal and higher FITC signal is expected when CD3 and HER2 Fabs are bound, and higher e450 with no FITC signal would be indicative of dissociated Fabs. When characterizing the modified SiTE, strong FITC signal was observed in the absence of AMD, and increasing e450 was observed in the presence of increasing AMD doses, confirming dose-dependent AMD-induced disassembly (FIG. 2F). Finally, circular dichroism experiment indicated that the secondary structure of the Fab was not affected (FIG. 2G). In all, these results confirmed the assembly and AMD-responsive disassembly of SiTE.

### Example 2: AMD-Controllable Activity of SiTEs In Vitro

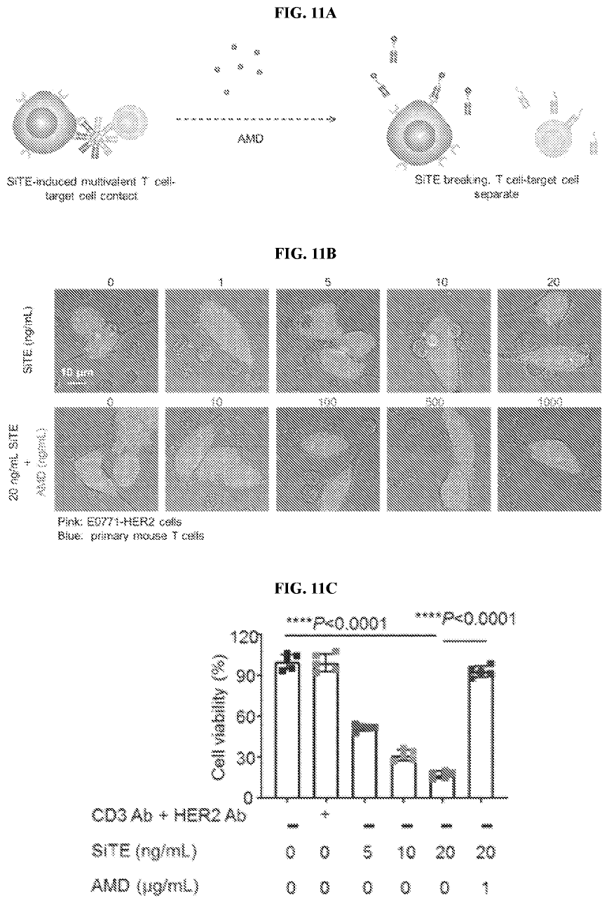

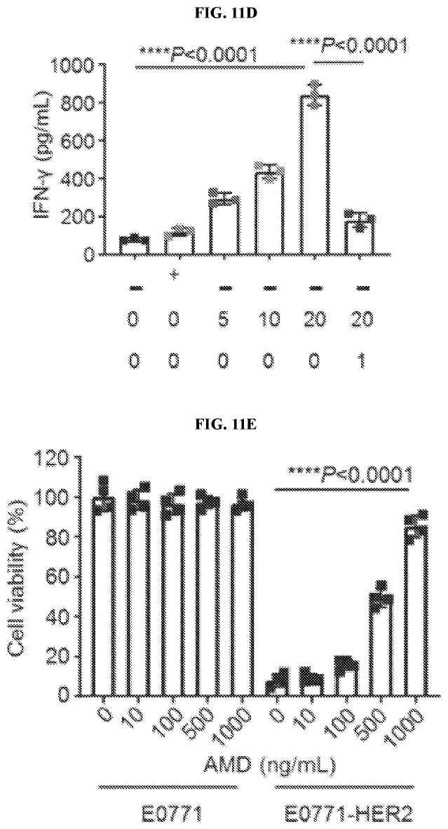

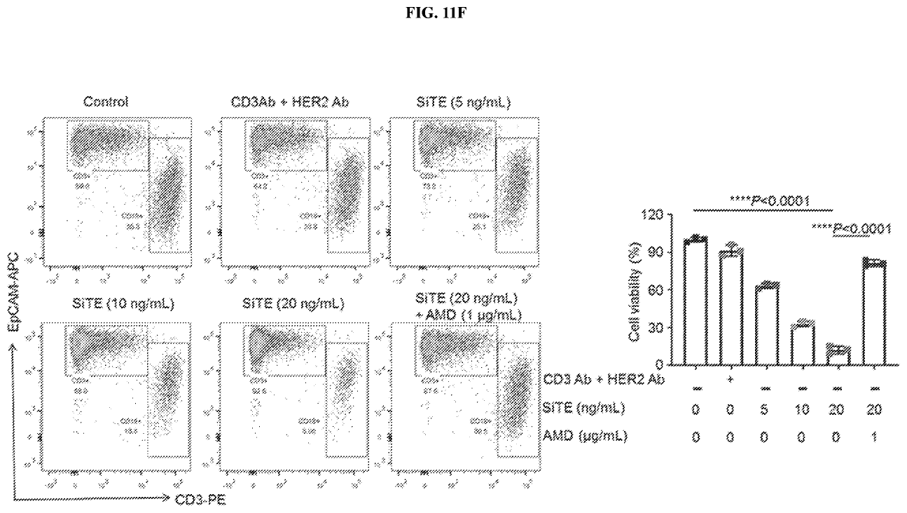

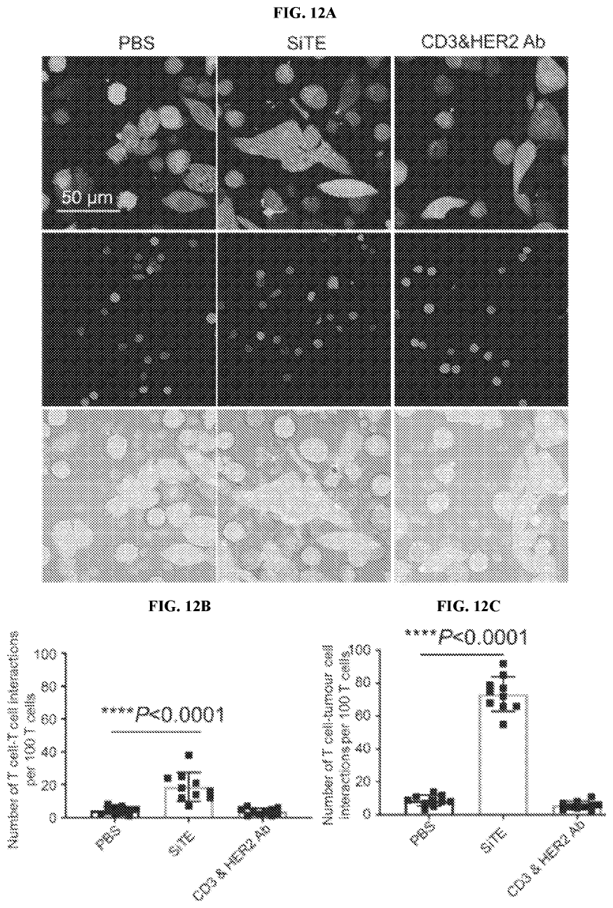

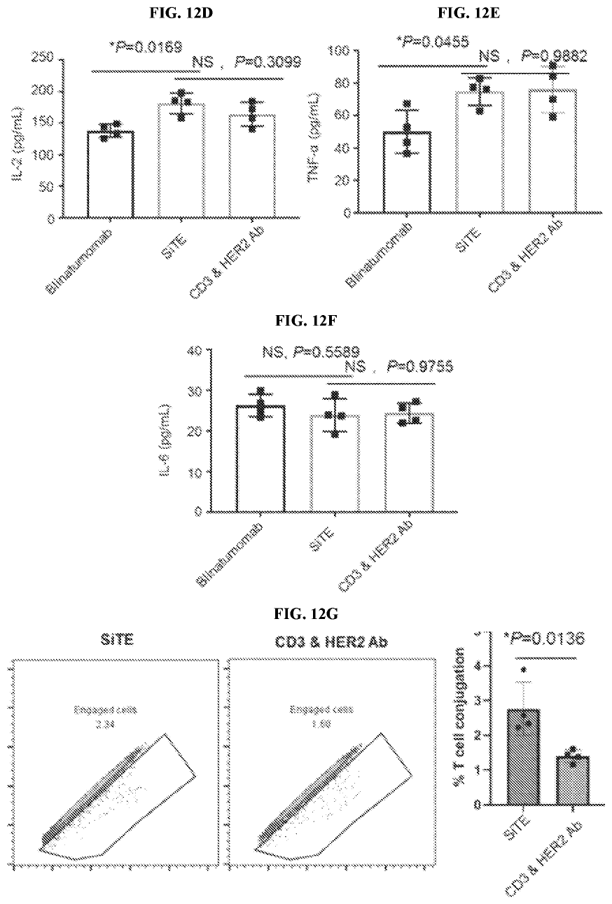

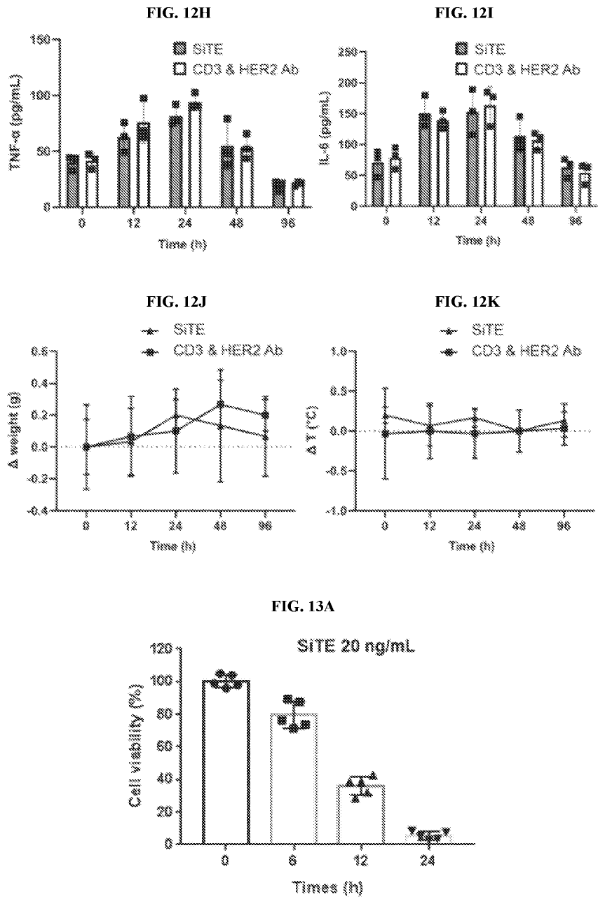

After synthesis and characterization of the AMD-responsive SiTE, the ability of SiTE to induce tumour cell-T cell engagement and tumour cell lysis was tested (FIG. 11A). E0771-HER2 cells were incubated with primary mouse T cells at a 1:1 ratio. Cells were then treated with SiTEs at varying concentrations for 4 h and observed under confocal laser scanning microscopy (CLSM) (FIG. 11B). In the untreated control group, cancer cells and T cells were randomly distributed with few T cells and cancer cells engaged. However, in groups receiving 5 ng/mL of SiTE, ~10% of T cells were engaged with tumour cells, and this rate increased to ~90% when cells were treated with 20 ng/mL of SiTE. These results demonstrate that SiTE engaged tumour cells and T cells in a dose-dependent manner. To ensure the specificity of this engagement. HER2 negative E0771 cells were observed and T cells treated with SiTE and found negligible T cell-cancer cell engagement (FIG. 9C). Further, treating the E0771-HER2 cells with free anti-CD3 Fab and anti-HER2 Fab before the addition of SiTE also did not result in T cell-cancer cell engagement (FIG. 9C), demonstrating that SiTE link T cells and cancer cells through the CD3 and HER2 surface antigens. Since the SiTE contains >20 Fabs, it is possible that it may induce T cell-to-T cell engagement and induce cytokine release. Upon investigation, it was found that about 73.5% of T cells were in contact with tumour cells and about 18.7% of T cells were in contact with T cells (FIGS. 12A-12C). The SiTE induced more cancer cell-T cell interactions than T cell-T cell interactions potentially due to the lower Kd value of the SiTE to HER2 antigen (7.25×10⁻¹¹ M) compared to the Kd of the SiTE to CD3 antigen (1.33×10⁻¹⁰ M) (FIGS. 10A-10B). Moreover, a 20% increase in released T cell cytokines IL-2 and TNF-α was found. These released cytokines may enhance the overall antitumour efficacy. To test if SiTE could induce T cell crosslinking in vivo and their effect on toxic cytokine release, SiTE was injected into healthy mice. After 24 h, mouse blood was collected and the T cell crosslinking levels and IL-6 and TNF-α levels were measured. It was found that SiTE induced about 2.5% of T cell crosslinking. However, the IL-6 and TNF-α concentration in the serum were comparable to those of a mixture of free CD3 and HER2 antibodies (FIGS. 12G-12i), indicate low toxicity. SiTE treatment also did not lead to body weight loss or high fever (FIGS. 12J-12K). Next, it was tested if AMD can disassemble SiTE and thus act to “break” T cell-tumour cell engagement. T cells and E0771-HER2 tumour cells were mixed (1:1 ratio) and treated with 20 ng/mL SiTE for 4 h to induce T cell-tumour cell engagement. After the addition of varying concentrations of AMD, it was found that AMD greatly reversed SiTE-induced engagement (FIG. 11B). A 100 ng/mL AMD treatment induced ~40% separation of conjugated cells while a 1 μg/mL AMD treatment resulted in nearly 100% separation, indicating an AMD dose-dependent disengagement. These results demonstrate that SiTE induced T cell-cancer cell engagement can be quickly broken with the addition of AMD.

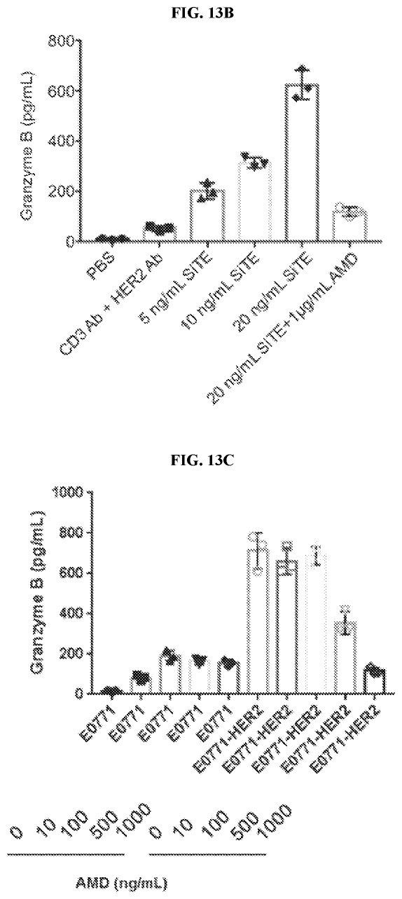

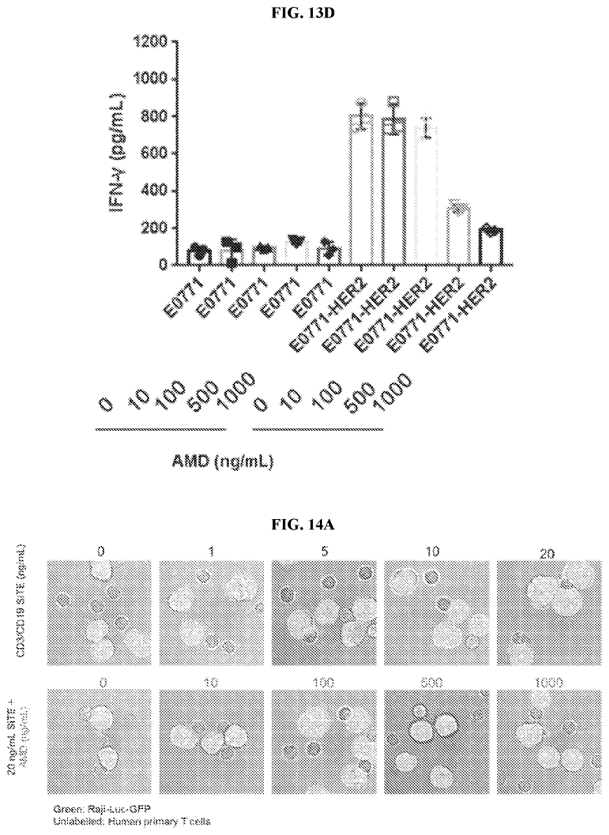

Next, it was investigated if the engagement of T cells and tumour cells by SITE induces tumour cell lysis. E0771-HER2 cells and mouse primary T cells were mixed at a 1:1 ratio and treated with varying concentrations of SiTE (5-20 ng/mL), a 1:1 mixture of CD3 Fab and HER2 Fab, or a mixture of 20 ng/mL SiTE with 1 μg/mL AMD. After 24 h, E0771-HER2 cell viability was measured (FIG. 11C). The cells treated with a mixture of CD3 Fab and HER2 Fab did not show tumour cell lysis, and the cell viability was comparable to untreated controls. However, 5 ng/mL SiTE treatment induced ~50% E0771-HER2 cell death, and cell death increased to ~90% when cells were treated with 20 ng/mL SiTE. In contrast, the group treated with 20 ng/mL SiTE along with 1 μg/mL AMD had almost no tumour cell death compared to PBS, demonstrating that AMD can prevent SiTE induced cancer cell lysis. In a similar experiment, SiTE treatment did not induce any toxicity to HER2 negative E0771 cells over a range of concentrations (FIG. 11E). Moreover, it was found that pre-treating the cells with free CD3 Fab and HER2 Fab can prevent the tumour cell lysing ability of SiTE (FIG. 9D). These results demonstrated that SiTEs induced antigen-specific cancer cell lysis. It was also found this specific cancer cell lysis induced by SiTE increases over time (FIG. 13A). Enzyme linked immunosorbent assays (ELISAs) for interferon-γ (IFN-γ) and granzyme B further confirmed SiTE-induced cancer cell killing and AMD-responsive dysfunction of the SiTEs (FIG. 11C and FIGS. 13B-13D). Together with flow results (FIG. 11F), it was demonstrated that the SiTE-induced cancer cell killing can be controlled in vitro using AMD.

Finally, to observe if the SiTE could be applied to other target cancers, SiTE was designed with anti-human CD3 Fab and anti-human CD19 Fab and tested their ability to induce T cell-cancer cell engagement, cancer cell lysis, and AMD-responsiveness (FIGS. 14A-14C). Comparable T cell-cancer cell engagement, tumour cell lysis, and AMD-dependent disassembly was found in the CD19-targeted SiTE, indicating the versatility and broad potential for this SiTE strategy. In all, these results demonstrate the efficacy of SiTE for tumour cell lysis in vitro and validate the ability of AMD to stop SiTE activity.

### Example 3: AMD-Controllable Avidity of SiTEs In Vivo

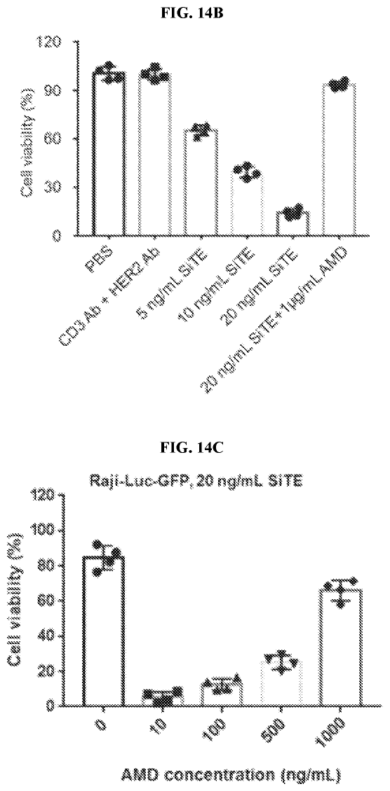

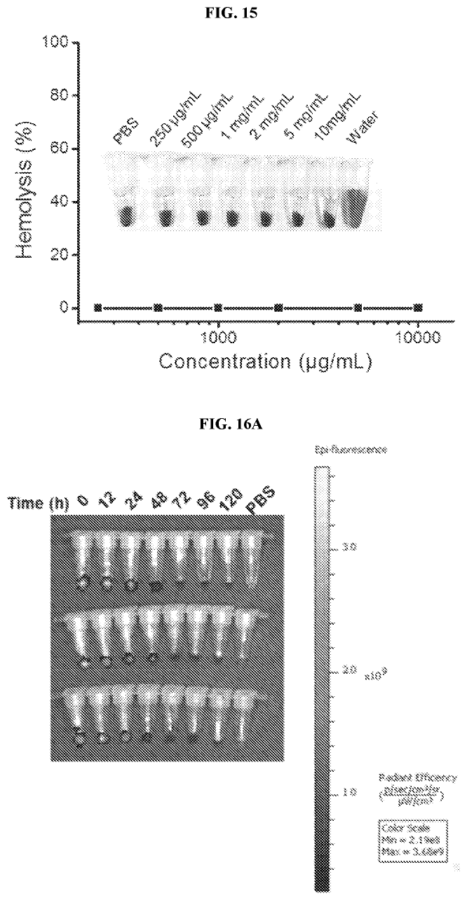

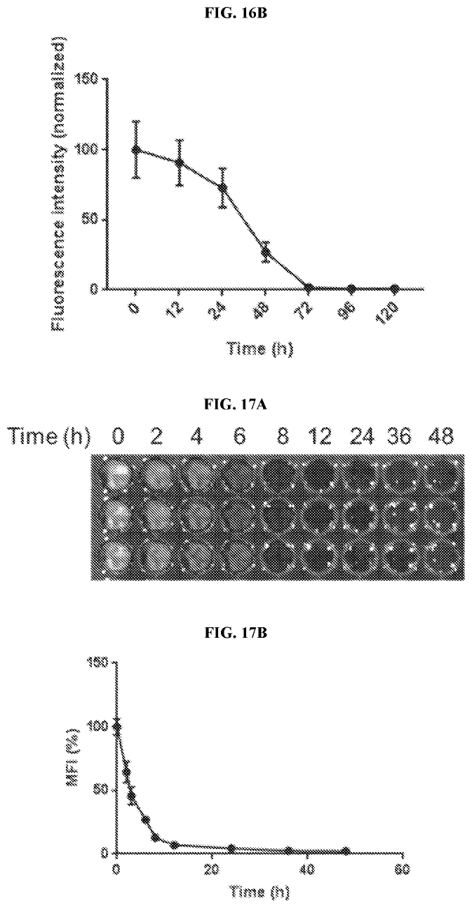

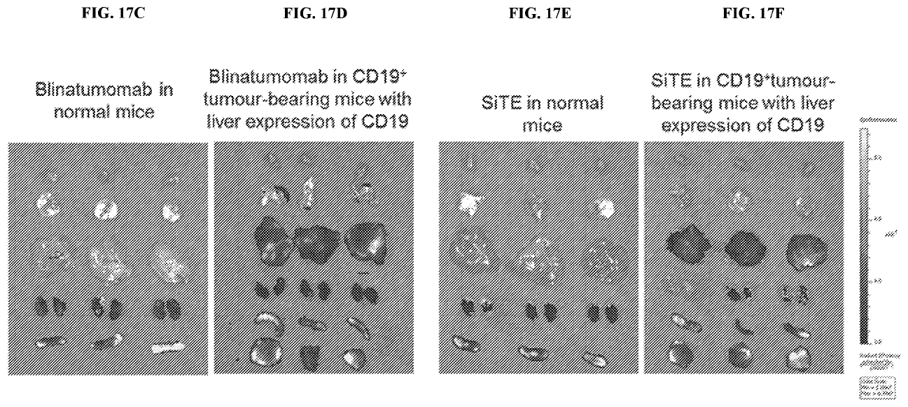

Encouraged by the results from the in vitro experiments, the controllable activity of SiTE was evaluated in vivo. To first assess safety for in vivo experiments, SiTE were incubated with red blood cells and were found to cause negligible red blood cell lysis at concentrations as high as 2 mg/mL (FIG. 15). As several studies demonstrated that PEGylation can greatly improve the blood circulation of many protein-based drugs, it was next investigated if the supramolecular linker in SITE increased blood circulation time (FIGS. 16A-16B). The results demonstrated that SiTE has a half-life of around 24 h, and that ~25% of SiTE remained in blood 48 h post-infusion. As compared to Blinatumomab, a traditional BiTE with a short half-life of 2.11 hours, SiTE exhibited an enhanced blood circulation time, which is important for anti-tumour applications. In the clinic, Blinatumomab may naturally possess a switch off mechanism since it can be quickly cleared from the blood due to its short half-life. However, it was found that even though Blinatumomab can be quickly cleared after i.v. injection (FIGS. 17A-17B), it accumulated greatly in the spleens of healthy mice with a humanized immune system (FIG. 17C). Moreover, high accumulation of Blinatumomab in the tumours, livers, and spleens (FIG. 17C) was also observed in Raji tumour-bearing humanized mice that express human CD19 antigen in the liver. It was also analyzed if the biodistribution of SiTE both in healthy NSG mice and in Raji tumour-bearing humanized NSG mice that express CD19 antigen in the liver. The SiTE possessed a similar biodistribution pattern to Blinatumomab in both groups of mice (FIG. 17D). The potential toxicity induced by such high accumulation of both Blinatumomab and the SiTE makes a switch off mechanism necessary to mitigate such toxicity.

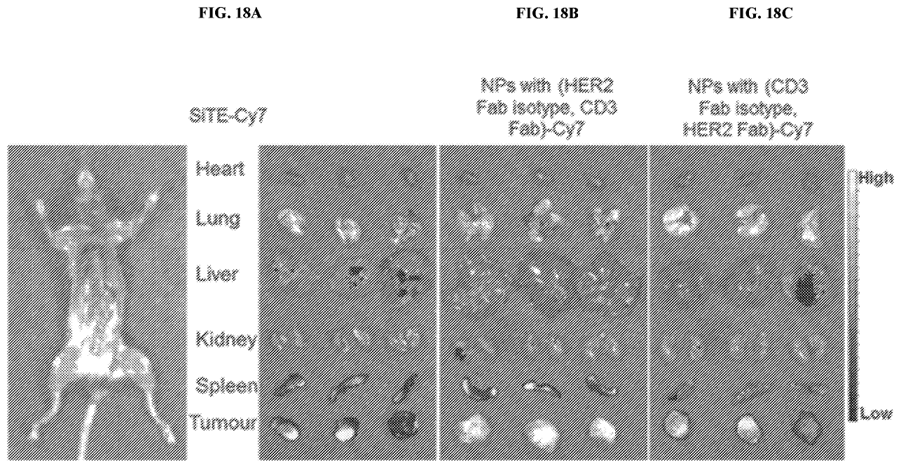

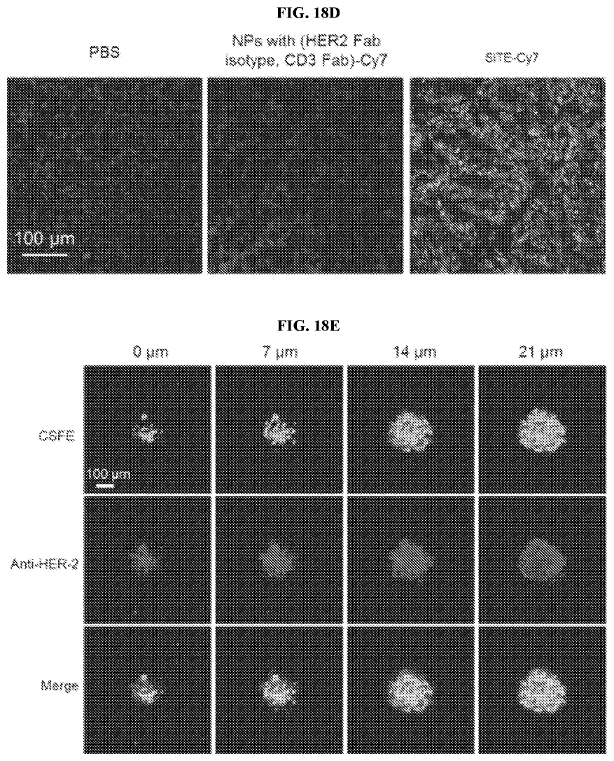

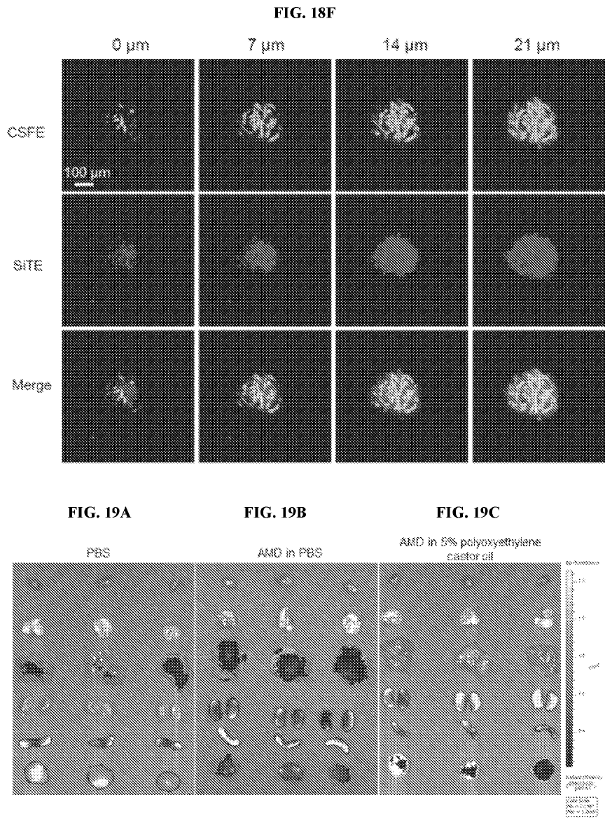

It was next examined if SiTE can target tumour tissues in vivo. A E0771-HER2 tumour model was first constructed before injecting the mice i.v. with Cy7-labeled SiTE (Cy7-labeled on CD3 Fab), a Cy7-labeled Fab nanoparticle made of 50% HER2 Fab isotype and 50% CD3 Fab (Cy7-labeled on CD3 Fab), or a Cy7-labeled Fab nanoparticle made of 50% CD3 Fab isotype and 50% HER2 Fab (Cy7 labeled on the CD3 Fab isotype) at 0 h. After 24 h, the mice were euthanized and the Cy7 signal in different organs and tumour was measured (FIGS. 18A-18F). The results showed that the SiTE mainly accumulates in the tumour and spleen. However, the nanoparticle with a HER2 Fab isotype mainly accumulates in the spleen. This is because CD3 Fab can target T cells in the spleen (FIG. 18B). The nanoparticle with a CD3 Fab isotype, however, mainly accumulates in tumour tissue. These results demonstrate that the presence of HER2 Fab in SiTE can help to target tumour tissue (FIG. 18C). Moreover, it was confirmed that SiTE can effectively penetrate and accumulate in deep tumour spheroid (FIGS. 18D-18F).

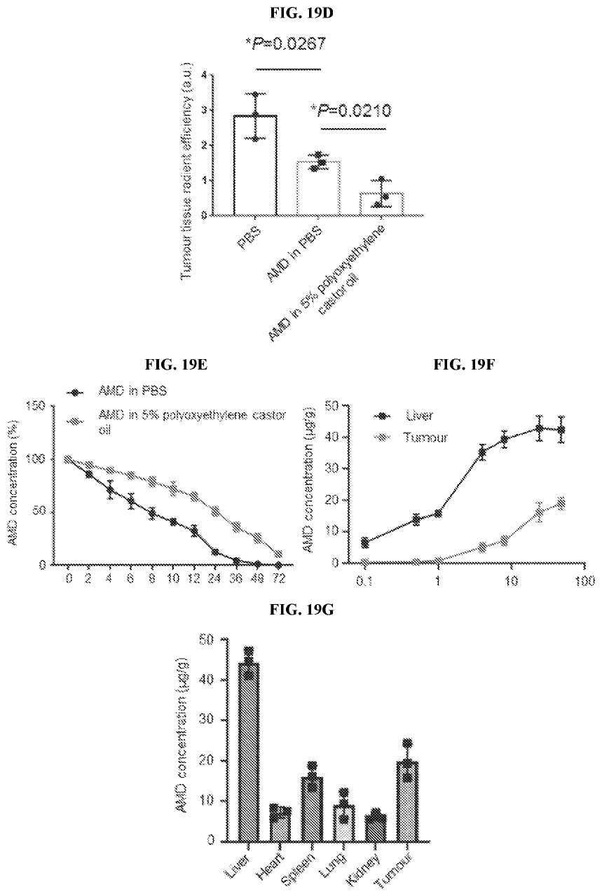

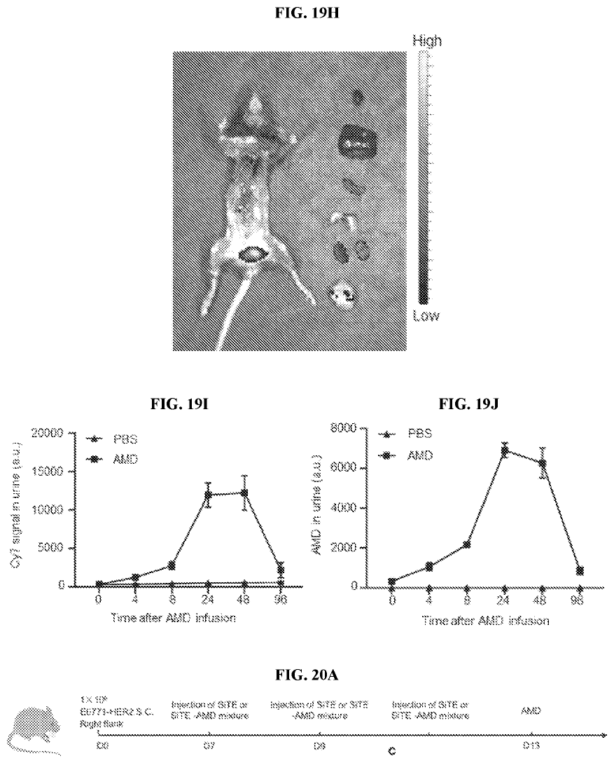

Next, determination as to whether AMD can be used to break down the SiTE in vivo was sought. Cy7 was modified to the CD3 Fab and the Fab assembled with HER2 Fab to formulate SiTE-Cy7. SiTE-Cy7 was i.v. injected to E0771-HER2 tumour-bearing mice at 0 h. At 18 h post-SiTE-Cy7 injection, PBS, AMD dispersed in PBS or AMD dispersed in 5% polyoxyethylene castor oil was i.v. injected to mice. Mice were euthanized 6 h later and the Cy7 signal in different organs and tumour was measured using an in vivo imaging system (IVIS). As shown in FIGS. 19A-19J, the SiTE-only group showed fluorescence signal in both the tumour and spleen. However, it was found that AMD dispersed in PBS didn't induce a complete breakdown of the SiTE, with about a 46% decrease in Cy7 signal in the tumour (FIGS. 19A-19B). Without wishing to be bound by any theory, this may be attributed to insufficient tumour accumulation due to the short half-life of AMD (FIG. 19D). However, it was found that dispersing AMD in 5% polyoxyethylene castor oil can help to enhance the half-life of AMD to about 24 h and this can induce over 78% Cy7 signal decrease compared to PBS treatment (FIG. 19E). This is likely due to the SiTE disassembling in the presence of AMD, allowing the Cy7-CD3 Fab to separate from the tumour-targeting HER2 Fab. A kinetic study showed that AMD first reached liver and then distributed to major organs and tumour tissues (FIGS. 19F-19G). Moreover, a higher Cy7 signal was detected in the kidney (FIG. 19C), indicating that the disassembled free Fab undergoes clearance from the kidney (FIGS. 19H-19I). AMD was also detected to be cleared from the urine (FIG. 19J), this is consistent with clinic report. These results demonstrate the SiTE can accumulate in tumour tissue, and an infusion of the small molecule AMD can disassemble SiTE to release the CD3 Fab from the tumour tissue. Most supramolecular chemistry-based strategies for controllable chemotherapeutic release have only been tested in in vitro studies, and only a few in vivo controllable drug release strategies have been reported in this space. These previous investigations may have been limited to mostly in vitro studies because the developed technologies require internalization by cancer cells, and it is difficult to precisely deliver multiple cargos into the same cancer cells in vivo. However, in this study, the SiTE link T cells and cancer cells while retained on the cell surfaces, which allows AMD to bind and control the separation of anti-CD3 Fab from anti-HER2 Fab extracellularly. This advantage makes the supramolecular chemistry-based controllable SiTE system uniquely useful in vivo.

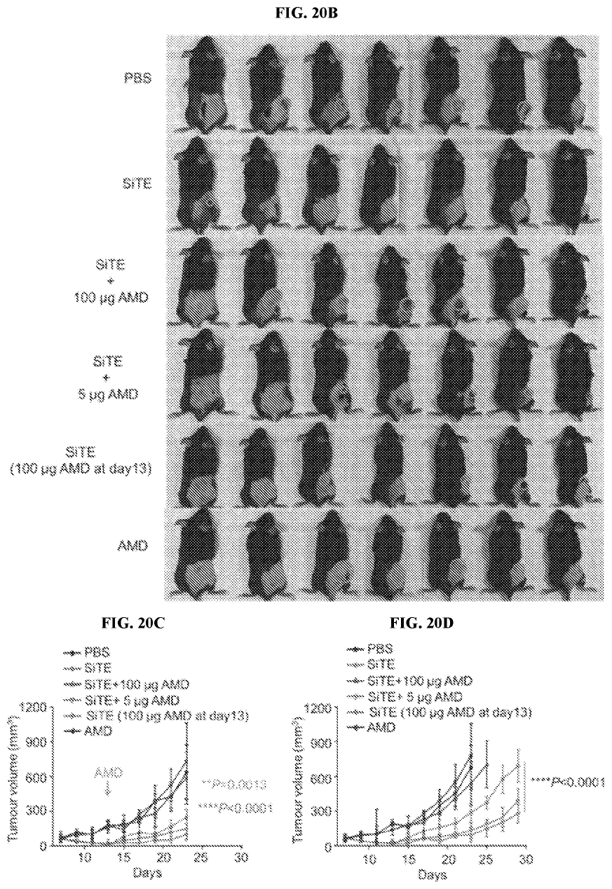

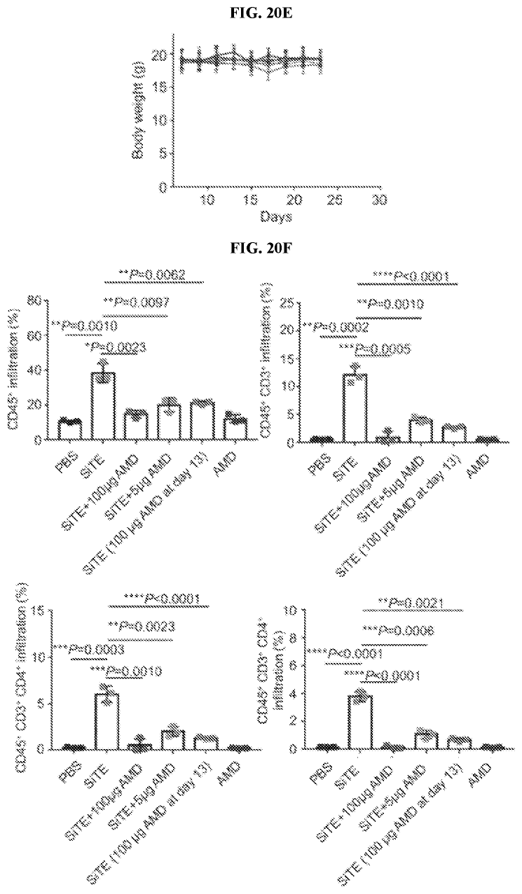

With confirmed tumour accumulation, it was next investigated if the target cell lysing capacity of SiTE can be controlled via AMD administration in vivo. Moreover, a E0771-HER2 tumour model was constructed, and mice were treated with three i.v. injections of PBS, SiTE (1 mg/kg), SiTE pre-mixed with 100 μg AMD, SiTE pre-mixed with 5 μg AMD, SiTE followed by one infusion of 100 μg AMD at day 13, or AMD (FIG. 20A). Tumour size and mouse weight were monitored. As shown in FIGS. 20B-20D, SiTE in the absence of AMD greatly inhibited tumour growth, as the tumour sizes in mice receiving SiTE reached ~70 mm³ at day 23 whereas untreated tumour sizes were ~650 mm, supporting that SiTE induces efficient target cell lysing (FIGS. 20C-20D). However, in mice treated with SiTE pre-mixed with AMD, the target cell lysing ability was attenuated, as tumour size was ~100 mm³ in mice treated with SiTE pre-mixed with 5 μg AMD, and ~600 mm³ in mice treated with SiTE pre-mixed with 100 μg AMD. These results suggest that pre-incubation of AMD with SiTE disassembled the SiTE and thus, reduced tumour inhibition. In mice receiving the SiTE followed by 100 μg AMD at day 13, tumour growth was greatly inhibited up until the AMD infusion. However, after the AMD infusion (day 13), tumour growth continued, resulting in final tumour sizes of ~650 mm³ at day 29 (FIG. 20D). These results demonstrate that the target cell lysing capacity of SiTE can be controlled using AMD.

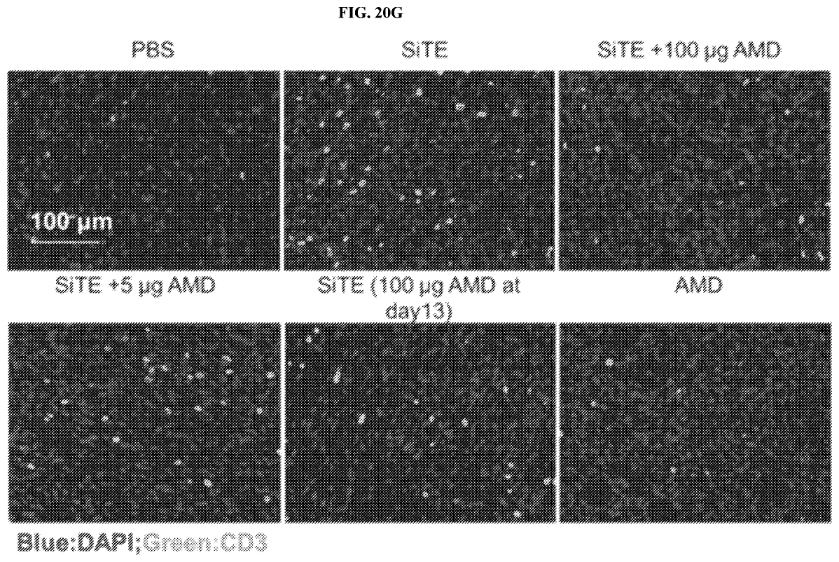

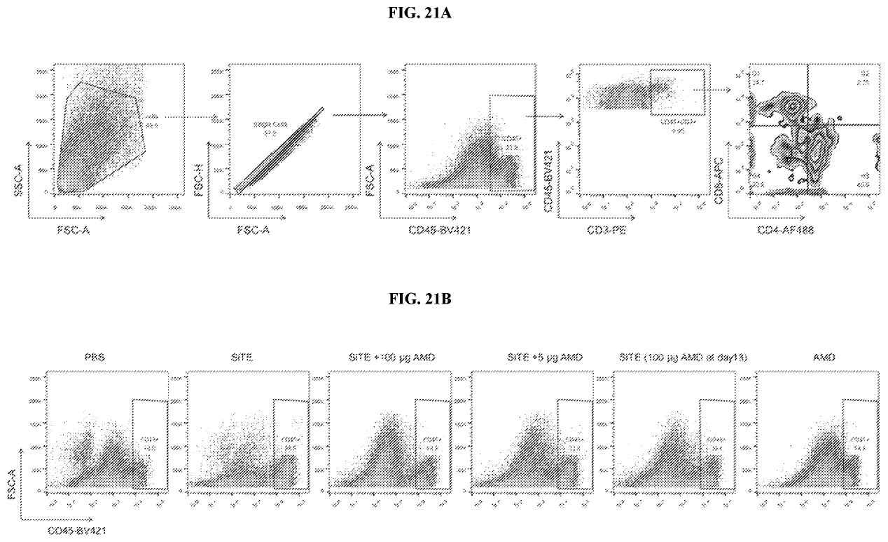

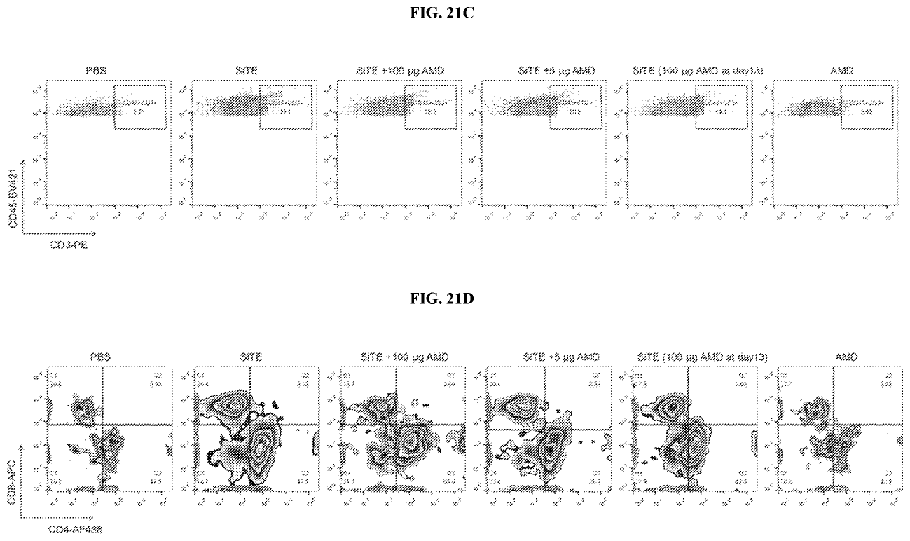

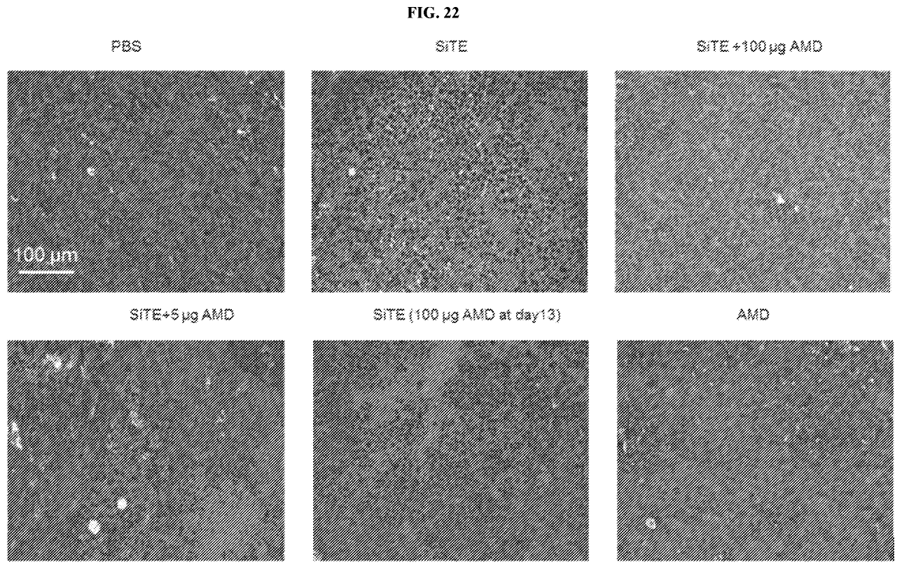

Minimal changes in mouse weight were observed across the different groups (FIG. 20E), likely because HER2 antigen is not expressed on normal mouse cells, preventing toxicity from on-target, off-tumour activity. At day 23, all mice were euthanized and tumour tissue was collected to determine total lymphocyte and CD3⁺ T cell infiltration using flow cytometry and immunofluorescence imaging. It was found that both CD45⁺ lymphocytes and CD3⁺ T cells were greatly increased in the tumour tissue of mice treated with SiTE compared to PBS-treated mice (FIGS. 20F-20G and FIGS. 21A-21D). However, groups receiving SiTE pre-mixed with AMD displayed lower rates of immune cell infiltration into tumour tissue, and the group receiving an infusion of AMD after SiTE treatment saw minimal infiltration as well, demonstrating that the AMD infusion likely reversed any infiltration induced by the SiTE. Further, histology experiments showed that the SiTE induced substantial tumour cell death in E0771-HER2 tumour tissue (FIG. 22) and AMD can greatly control the activity of SiTE in vivo.

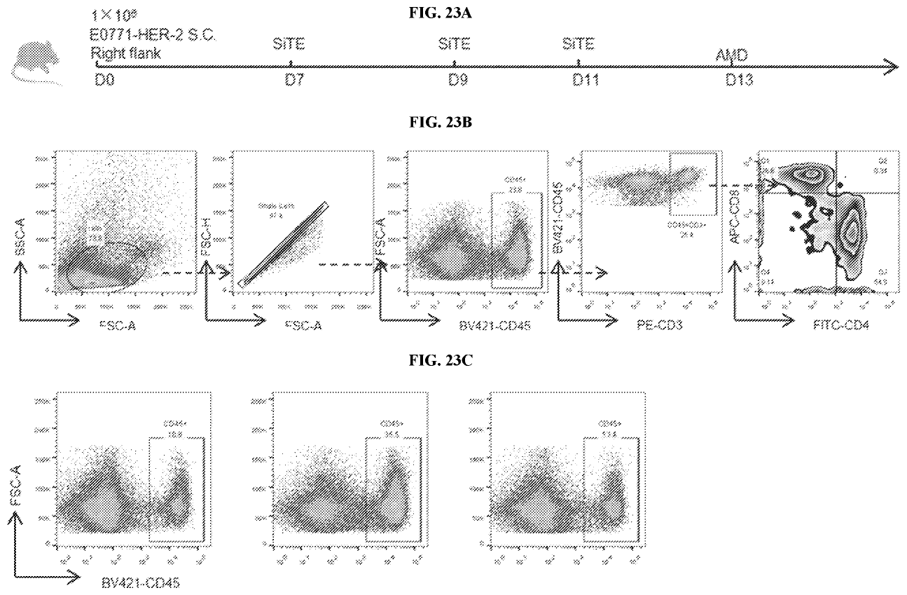

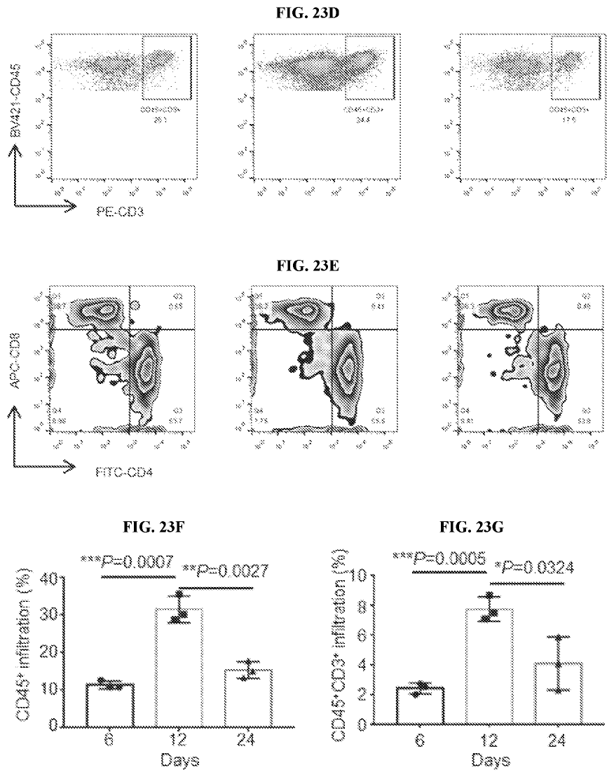

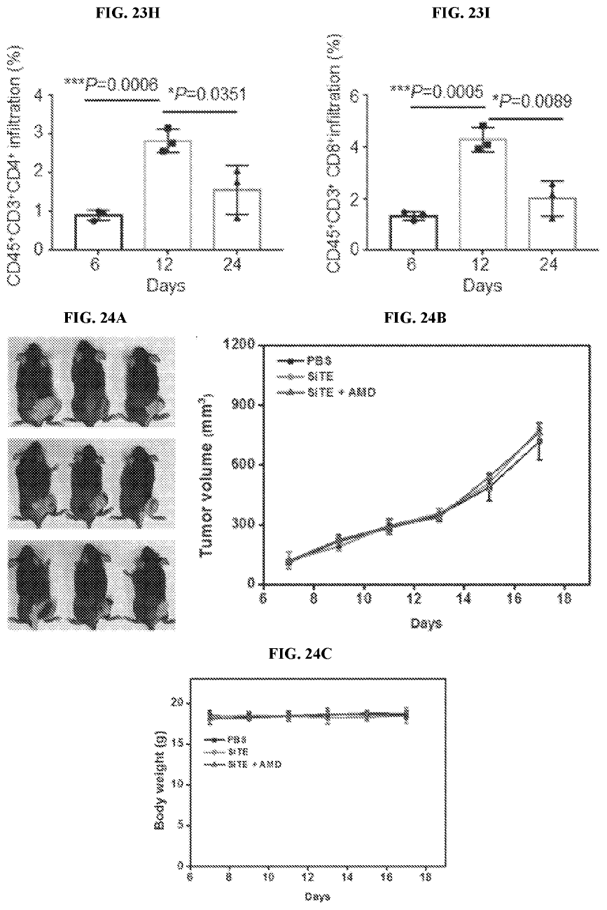

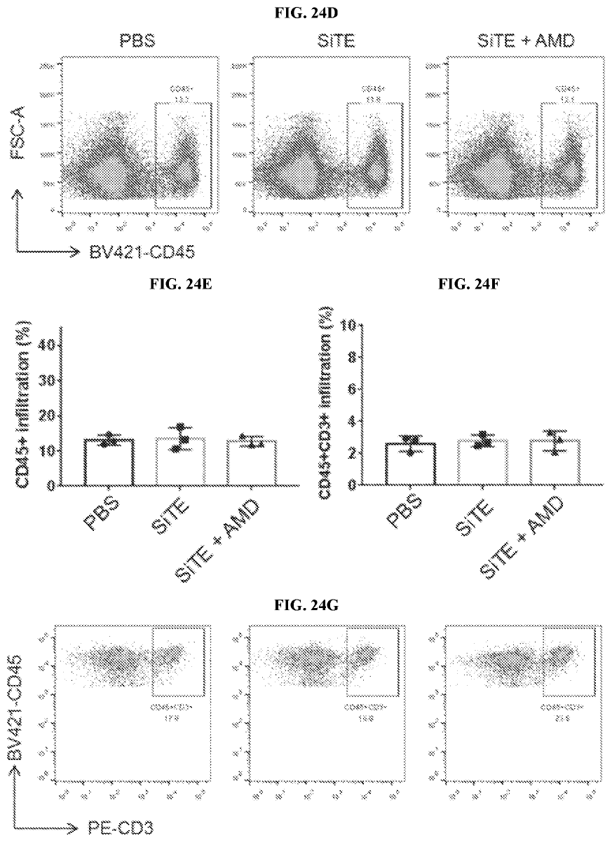

After observing the ability of SiTE to induce tumour infiltration and the potential of AMD to reverse it, a separate animal experiment was performed to assess T cell infiltration in tumour tissue at different time points, including before and after an AMD infusion (FIGS. 23A-23I). The results showed that SiTE greatly improved T cell infiltration, but following AMD infusion, the number of T cells in the tumour tissue greatly decreased (FIGS. 23A-23I), further confirming the controllable activity of SiTE. Additionally, to confirm that this impact on the tumour tissue was HER2 specific, SiTE activity was evaluated in a HER2 negative E0771 tumour model (FIGS. 24A-24J). It was found that SiTE did not inhibit tumour growth, and T cell infiltration in HER2 negative E0771 tumour tissue also was not affected when mice were treated with SiTE and/or AMD. These results show that tumour cell-T cell engagement and tumour cell lysis by SiTE is HER2 antigen-specific. Altogether, these results demonstrate that SiTE can substantially and specifically inhibit tumour growth in vivo and that this can be reversed using AMD.

### Example 4: AMD Alleviates the On-Target, Off-Tumour Toxicity of SiTE In Vivo

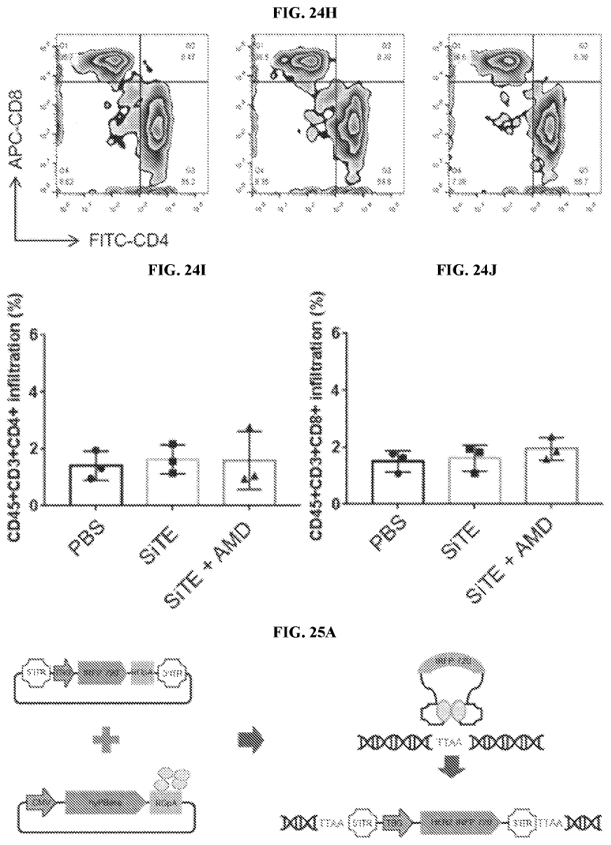

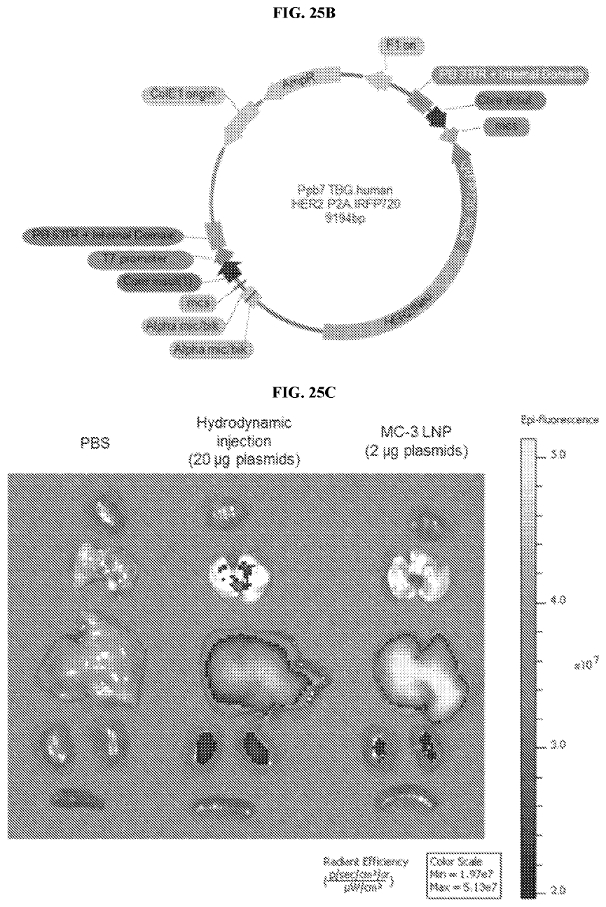

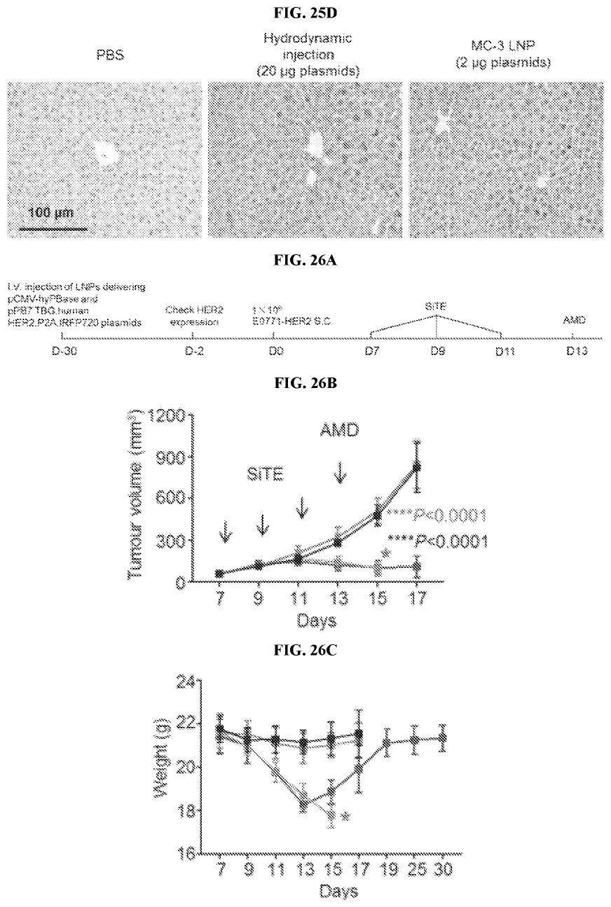

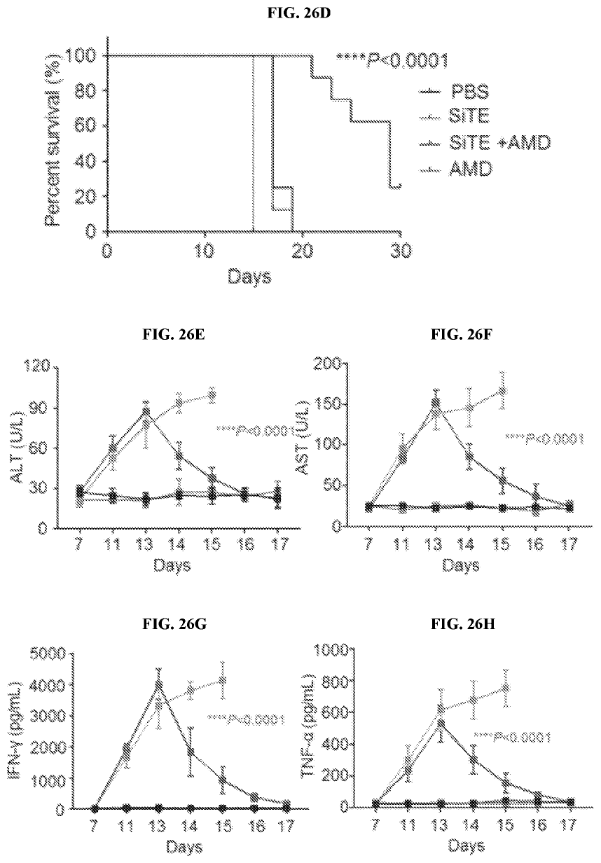

Recently, a mouse model was reported with stable human HER2 antigen expression in the mouse liver. They used a hydrodynamic injection method to deliver a transposase plasmid and transposon plasmid encoding human HER2 antigen and an IRFP reporter to the mouse liver, and they found that the HER2 antigen was then stably expressed on mouse liver cells. Here, the construction of this mouse model was modified and two plasmids were delivered using a lipid nanoparticle (FIGS. 25A-25D). Notably, higher IRFP and HER2 expression were found in mice treated with LNPs at a lower dose of plasmids compared to those treated with plasmids via the hydrodynamic injection method (FIGS. 25A-25D). Next, a E0771-HER2 tumour model was constructed in mice with HER2 expression in the liver (FIG. 26A). After tumour size reached 100 mm, mice were i.v. injected with PBS, SiTE, or free AMD. Half of the group receiving SiTE on days 7, 9, and 11 also received AMD on day 13. The mice were then monitored for tumour growth, body weight, and survival (FIGS. 26B-26D). The results showed that SiTE greatly suppressed tumour growth, but they also induced substantial weight loss. This is likely due to off-tumour toxicity caused by the HER2 antigen expression in mouse liver cells resulting in liver cell lysis. Interestingly, on day 13, when half of the SiTE treated mice received 100 μg AMD to disassemble SiTE, the group of mice receiving AMD had gradual increases in body weight and recovered to a normal range while those without an AMD infusion had to be euthanized due to continued weight loss. In the SiTE+AMD group (FIG. 26D), the tumours were growth to bigger and the mice were euthanized when the tumour size reached 700 mmi.

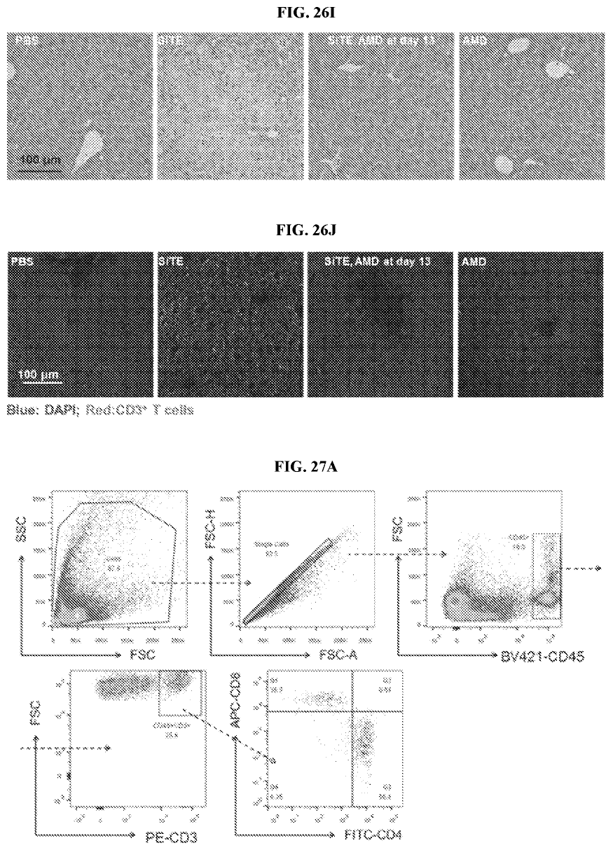

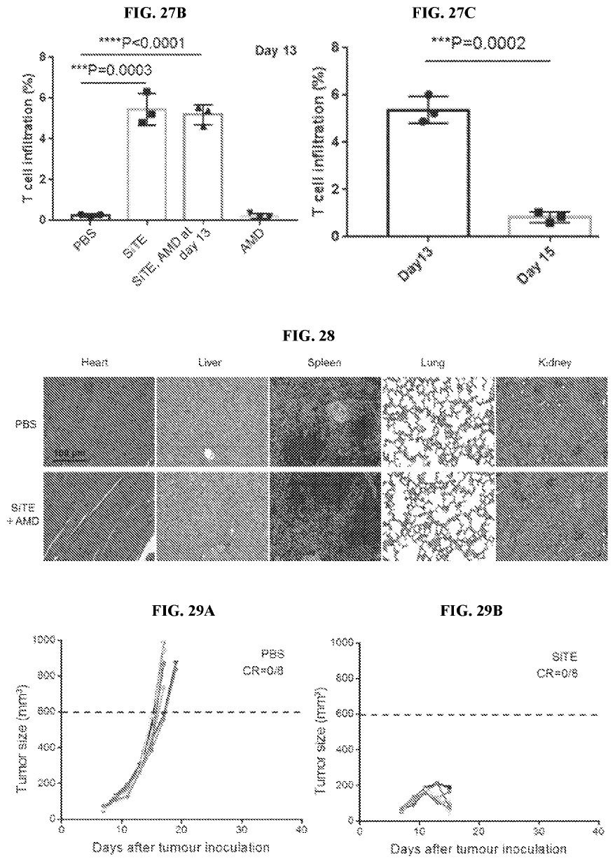

To better characterize the weight loss seen in the SiTE only treatment group, a separate animal experiment was conducted to analyze the alanine aminotransferase (ALT), aspartate aminotransferase (AST). IFN-γ, and TNF-α levels in the mouse blood (FIGS. 26E-26H). The results showed that SiTE treatment increased ALT and AST levels in the blood, indicating severe liver cell damage. Moreover, IFN-γ and TNF-α levels in blood also increased in mice treated with SiTE (FIGS. 26G-26H), suggesting that SiTE induced T cell engagement and target cell lysis. However, ALT and AST returned to lower levels 24 h after AMD infusion, demonstrating that AMD can stop the liver cell damage caused by SiTE and that the response is rapid (FIGS. 26E-26F). Flow cytometry was also used to determine T cell infiltration levels in liver tissue before and after AMD infusion. It was found that treatment with SiTE greatly increased the number of T cells in liver tissue (FIGS. 27A-27C). However, infusion of AMD after SiTE treatment greatly decreased the number of T cells in liver tissue (FIGS. 27B-27C). Histology experiments further confirmed that SiTE caused severe liver cell damage, and AMD reversed this damage by decreasing T cell infiltration in the liver (FIGS. 26I-26J and FIG. 28). These results demonstrate that the on-target, off-tumour toxicity of SiTE can be controlled via AMD administration.

### Example 5: A High Doses of SiTE Elicits a Tumour-Specific Immune Response with Limited On-Target, Off-Tumour Toxicity

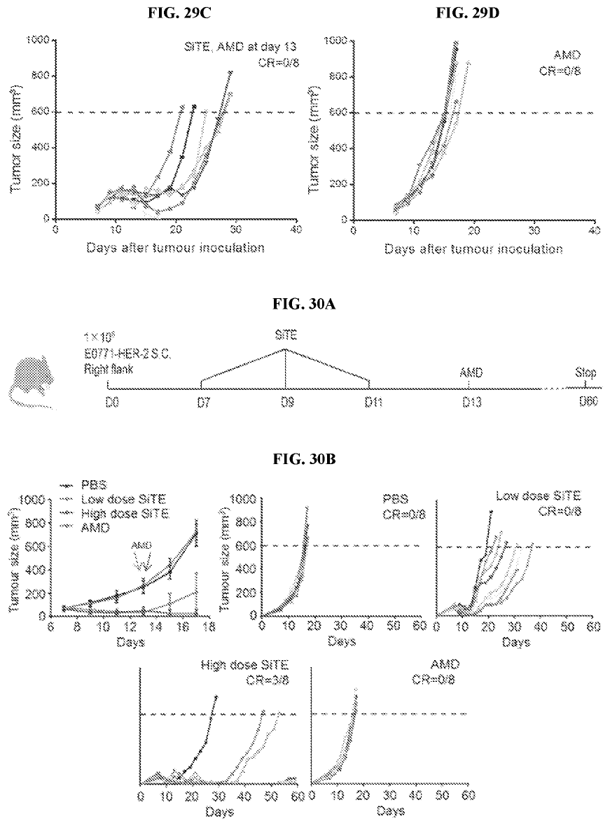

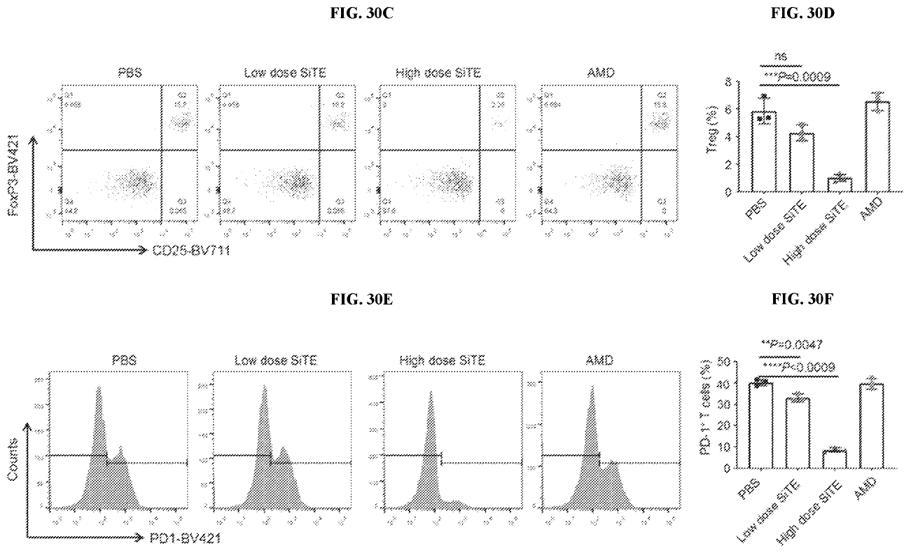

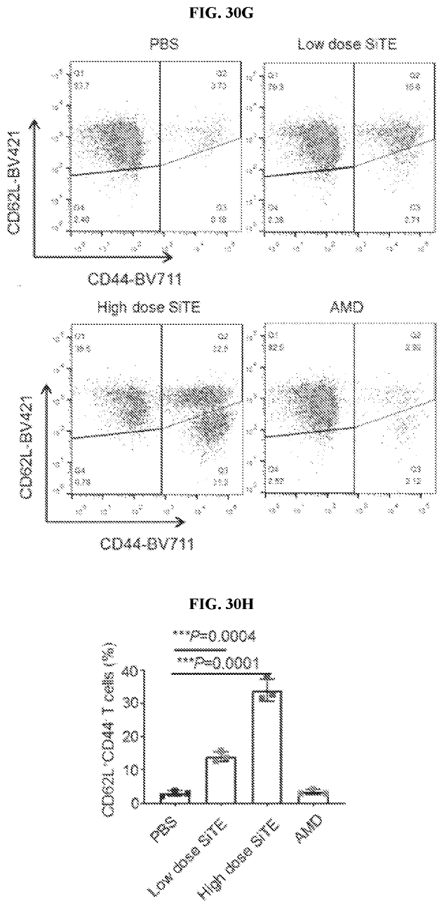

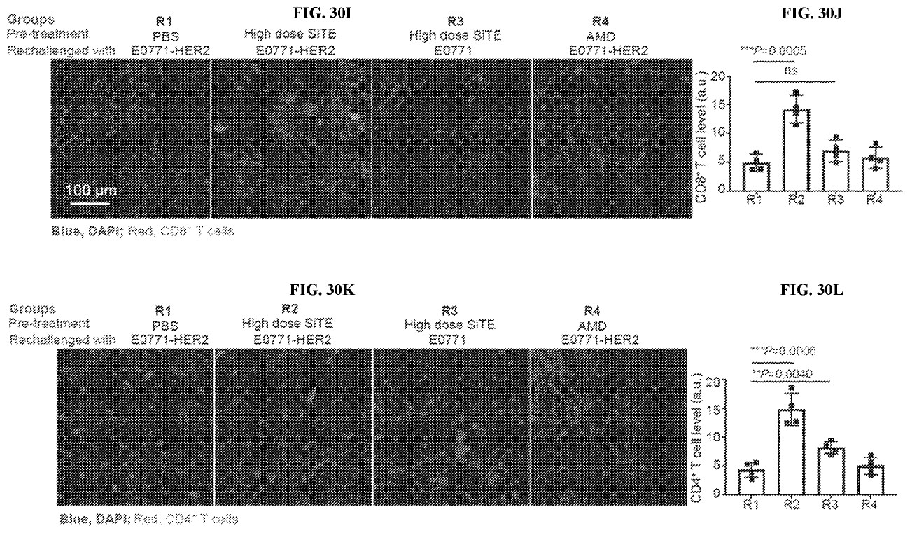

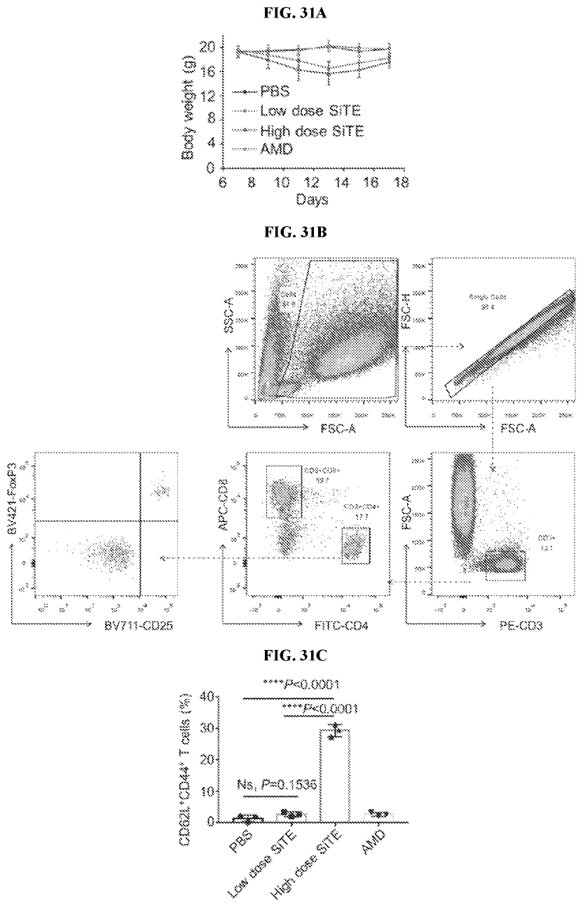

While it has been demonstrated that the off-tumour toxicity of SiTE can be controlled via an infusion of AMD to disassemble SiTE, it was found that tumour inhibition was also affected by AMD. Though SiTE stalled tumour growth, most tumours relapsed after treatment with an AMD infusion (FIGS. 29A-29D). This is likely due to AMD disassembling the SiTE and thus breaking the engagement of T cells and tumour cells. However, a key advantage of SiTE is their controllable activity to prevent off-tumour toxicity, which allows exploration of the use of higher doses of SiTE in further in vivo experiments. The E0771-HER2 mouse model was constructed with liver expression of human HER2 antigen. After the tumour size reached ~100 mm³, mice were treated with PBS, a low dose of SiTE (1 mg/kg), a high dose of SiTE (5 mg/kg), or AMD (in 5% polyoxyethylene castor oil) only (FIG. 30A). When mouse body weight decreased by more than 15%, AMD was administered to decrease the off-tumour toxicity (FIG. 31A). As shown in FIG. 30B, in mice treated with PBS, the tumours reached 600 mm³ at around day 17 and mice were euthanized. In mice that received the low-dose of SiTE, tumour growth was inhibited through day 13 when AMD was administered. However, most tumours continued to grow after the infusion of AMD to disassemble SiTE, and no mice in this group survived past day 60 (FIG. 30B). In mice treated with the high-dose of SiTE, tumour growth was greatly inhibited through day 13, and after receiving AMD, only 3 of 8 mice showed tumour relapse through day 60 with the majority of tumours remaining undetectable (FIG. 30B). Thus, the higher dose of SiTE allowed for continued anti-tumour activity in mice even after SiTE disassembly.

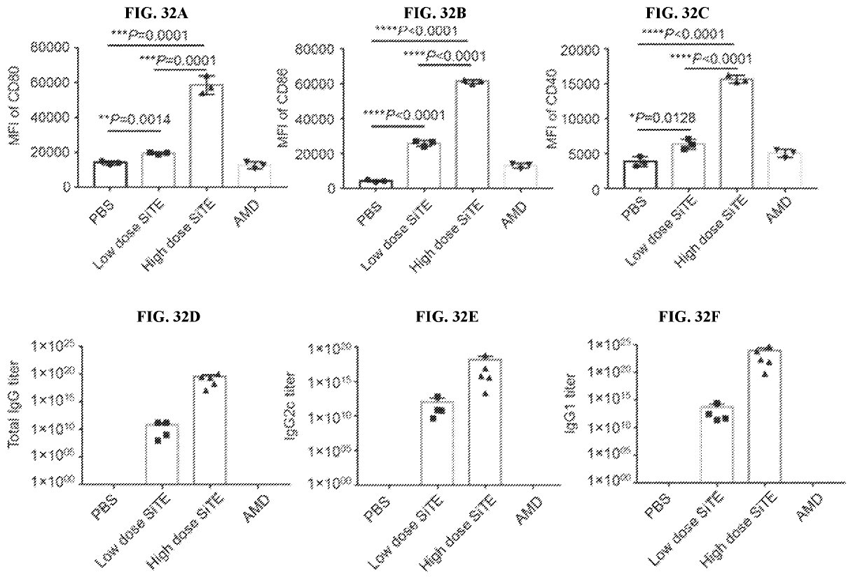

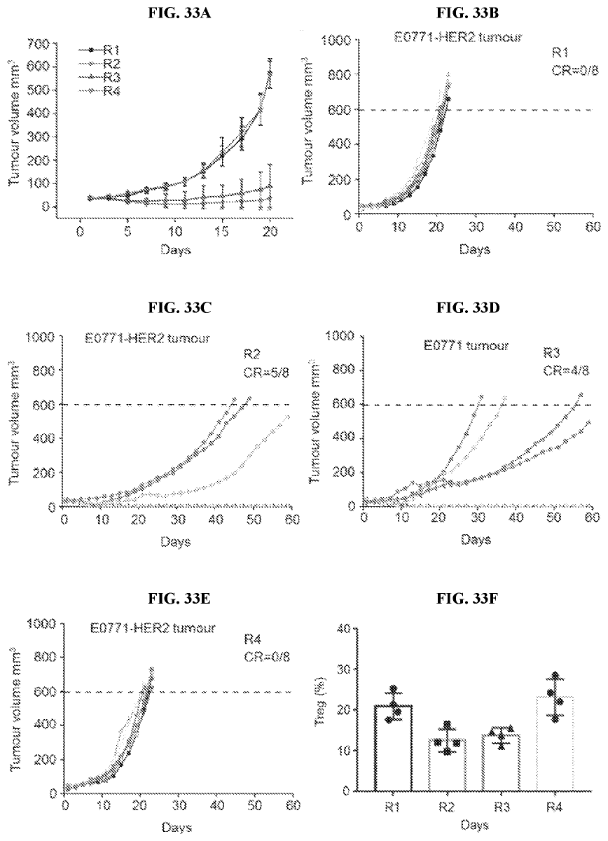

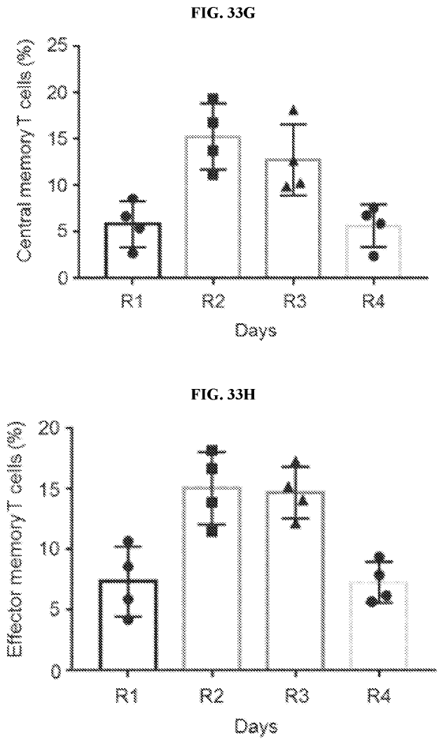

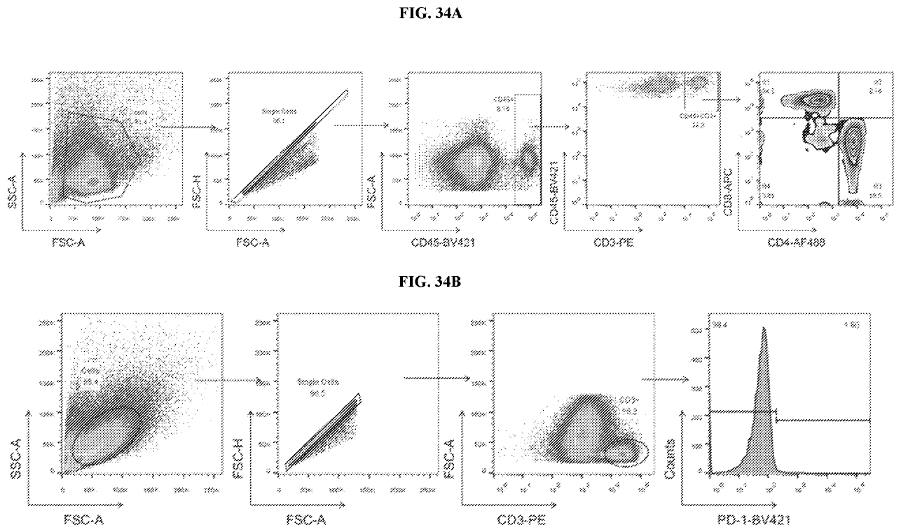

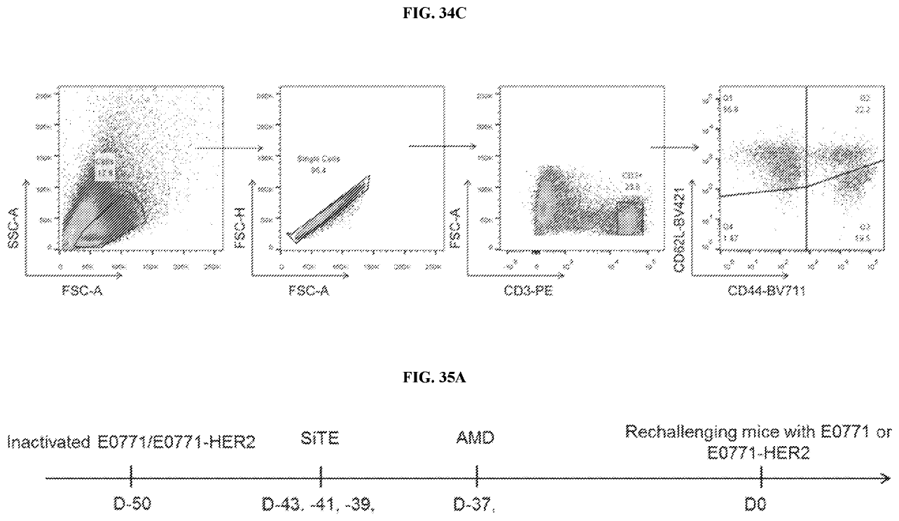

To better characterize this anti-tumour activity, an additional animal experiment was performed to analyze the immune microenvironment in tumour tissue. Using the same E0771-HER2 model and treatment groups detailed above, mice were euthanized at day 17 and tumour tissue was isolated. Flow cytometry results showed that the CD25⁺FoxP3+ regulatory T cell (Treg) population (FIGS. 30C-30D) and PD-1⁺ exhausted T cells (FIGS. 30E-30F) were greatly decreased in mice treated with a high dose of SiTE. However, mice treated with a low dose of SiTE showed only a slight decrease in these cell populations (FIGS. 30C-30F). It was also found that mice treated with a high dose of SITE greatly improved both CD44⁺CD62L⁺ central memory cells and CD44⁻CD62L⁻ effector memory cell populations, in tumour tissue (FIGS. 30G-30H and FIG. 31C). Moreover, the increased expression of co-stimulatory molecules, such as CD40, CD80, and CD86 on DC cells (FIGS. 32A-32C), and higher HER2-specific total IgG, IgG2c, and IgG1 levels were also detected in the high dose SiTE group (FIGS. 32D-32F). To explore this prolonged anti-tumour activity, a tumour cell re-challenging experiment was performed (FIGS. 33A-33H). 10⁶ E0771-HER2 or E0771 cells were injected into tumour-free mice from the high dose SiTE group, and PBS or AMD pre-treated mice challenged with E0771-HER2 cells were used as controls. In mice that had previously received a high dose of SiTE and were challenged with E0771-HER2, only 3 of 8 mice showed tumour growth, and tumour growth was much slower than in naïve mice. Surprisingly, when mice previously receiving high-dose SiTE were challenged with HER2 negative E0771, tumour growth was also inhibited, as only 4 of 8 mice showed tumour growth and the other mice were tumour-free (FIG. 33D). This demonstrates that tumour-specific immune memory was induced after treatment with a high dose of SiTE. However, PBS or AMD pre-treated mice did not show any ability to inhibit tumour cell growth in this re-challenge model. To further explore the anti-tumour activity in SiTE-treated mice, immune cell infiltration in the tumour tissue was analyzed (FIGS. 30I-30L and FIGS. 33F-33H). The results demonstrated that T cells readily infiltrated into tumour tissue in mice previously treated with SiTE. Most of the CD3⁺ T cells were PDI-negative in mice that had received a high dose of SiTE, and Treg cell number was also decreased in these mice compared with the CD3⁺ T cells in mice receiving no SiTE treatments (FIGS. 33F-33H, flow gating strategies are shown in FIGS. 34A-34C).

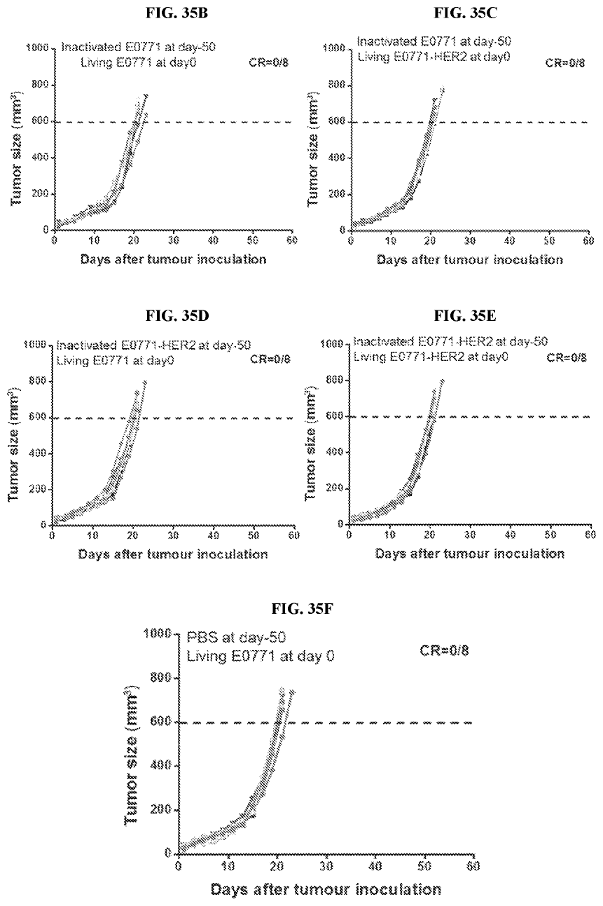

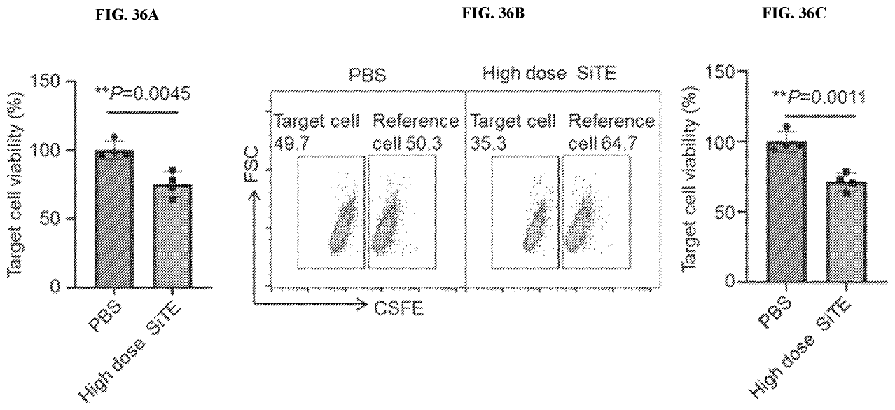

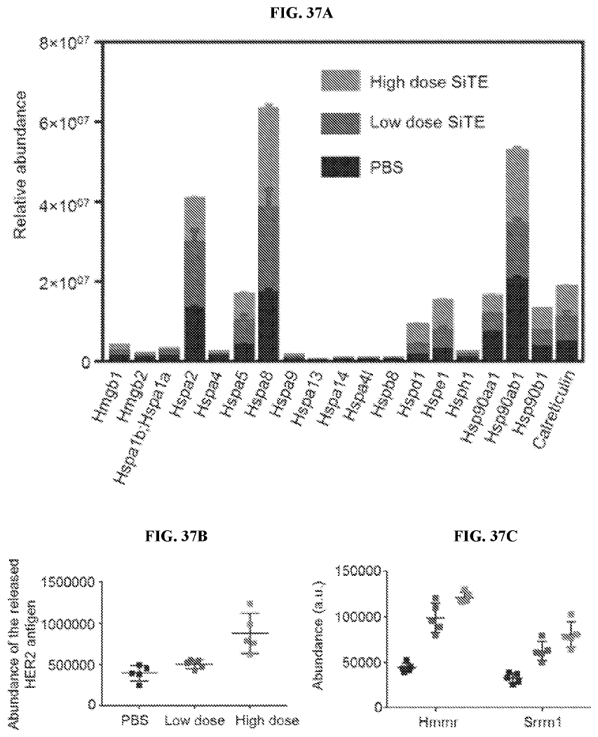

In order to better understand how the high dose of SiTE induces an in situ vaccination effect, two additional experiments were performed. It was found that inactivation of tumour cells with formaldehyde before injection into mice, and treating the mice with high doses of SiTE and AMD failed to provide the mice protection from both E0771 and E0771-HER2 cell rechallenging (FIGS. 35A-35F). Moreover, it was found that T cells isolated from high dose SiTE treated mice induced modest target tumour cell killing, compared to T cells isolated from PBS treated mice (FIG. 36A). Moreover, an in vivo killing assay also demonstrate high dose SiTE elicited tumour cell-specific immune response (FIGS. 36B-36C). These further support the contribution of the in situ vaccination effect in tumour growth inhibition and long term protection. Additionally, it was found that high doses of SiTE induced the release of higher levels of damage associated molecular patterns (DAMPs) from the tumour cells (FIG. 37A). Moreover, increased release of HER2 antigen and many mutation-derived neoantigens were also detected (FIGS. 37B-37C). These DAMPs and antigens can be taken up by DC cells and tumour antigen-specific T cell immune responses can be elicited. This can not only recognize HER2-positive E0771 cells, but can recognize the HER2-negative E0771 cells because of the shared neoantigen expression in both E0771 and E0771-HER2 cell lines. These results demonstrate that a high dose of SiTE elicited an in situ vaccination effect that can provide protection from tumour relapse.

### Example 6: SiTE Decrease Cytokine Release Syndrome and Neurotoxicity in a Humanized Immune System Mice Model

As it has been demonstrated that the SiTE strategy can be used to control the on-target, off-tumour toxicity in a mouse HER2 tumour model, whether this strategy can be used to control CRS and neurotoxicity was next assessed. CRS and neurotoxicity models were constructed in a humanized immune system mouse model when using CAR T cells to treat B cell lymphoma. In this study, it was investigated if the SiTE that targets human CD3 and human CD19 can be used to control on-target, off-tumour toxicity, CRS, and neurotoxicity in the humanized immune system mouse model.

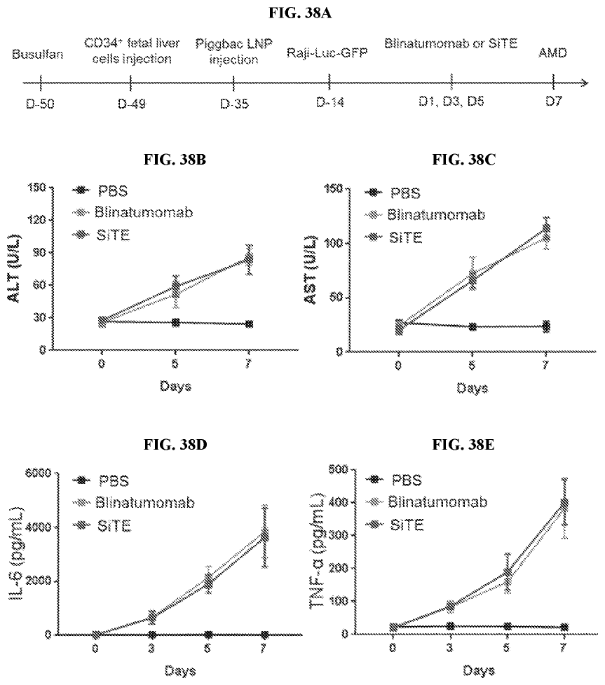

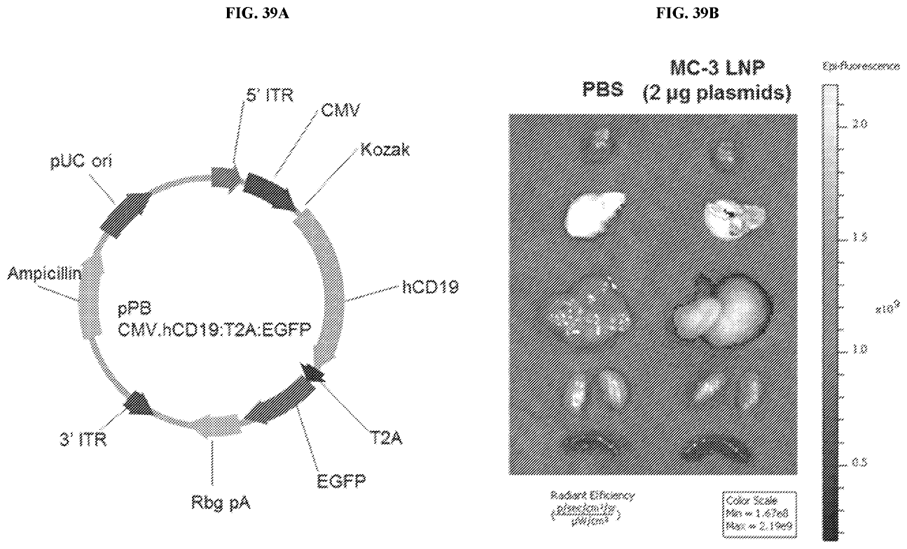

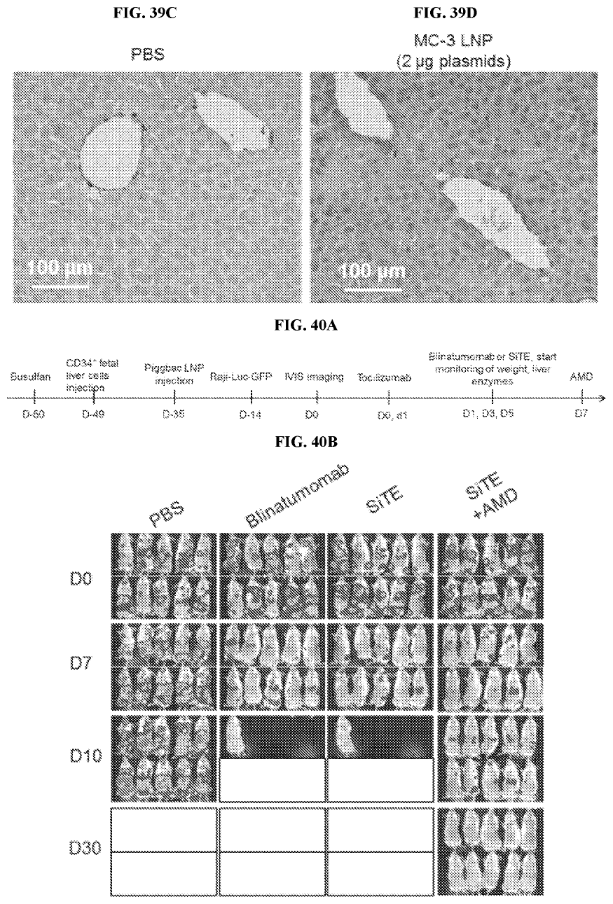

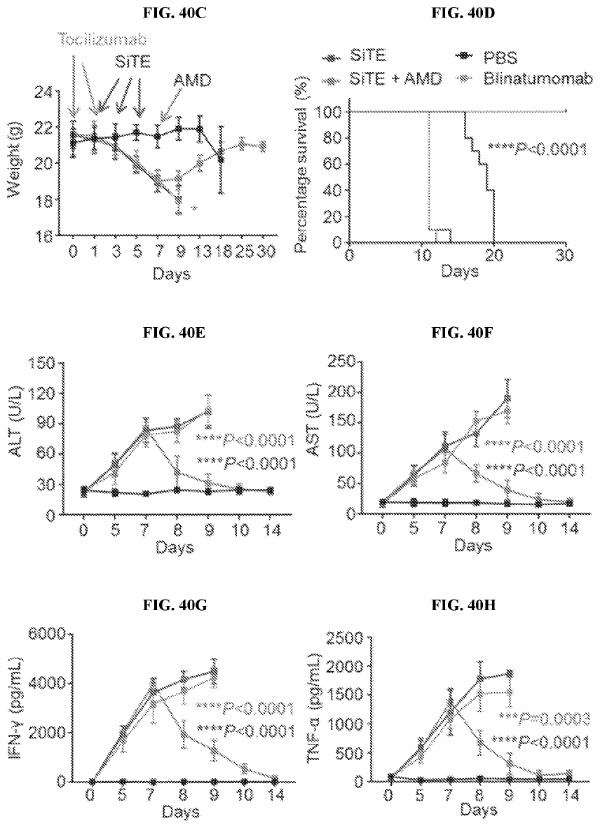

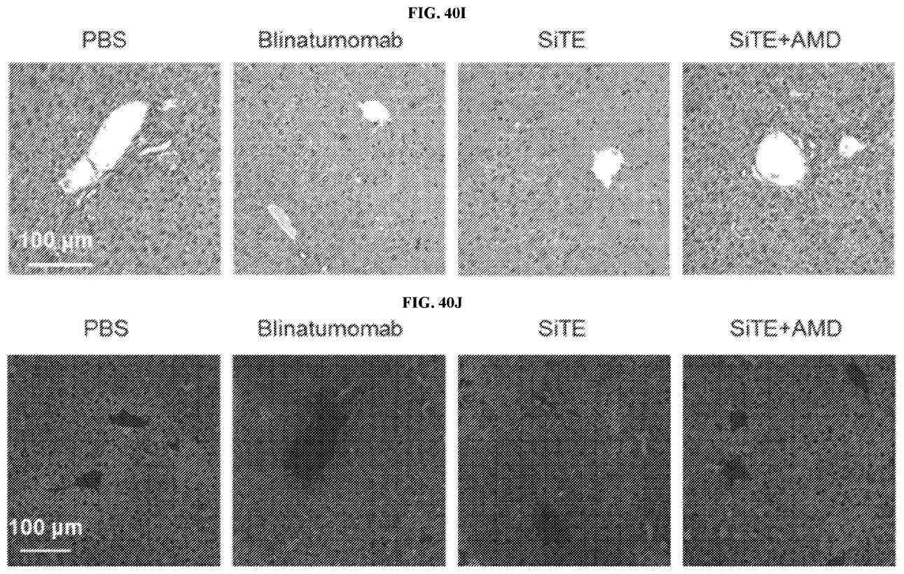

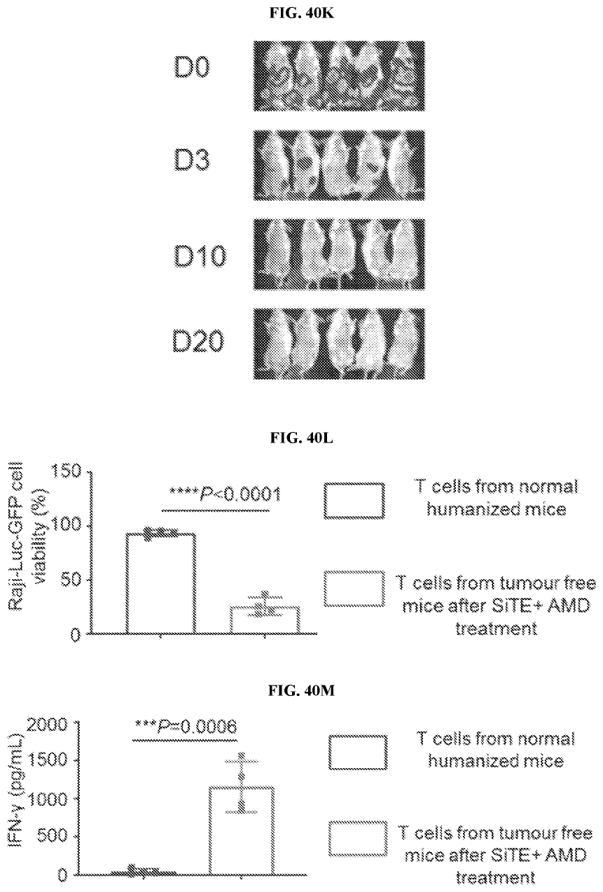

First, a humanized immune system mouse model was constructed by transplanting human CD34⁺ fetal liver cells into busulfan-treated mice (FIGS. 38A-38E). After that, human CD19 protein was expressed in the livers of these mice using a piggybac transposon system at day −35 (FIGS. 39A-39D). Raji cells, a human Burkitt's lymphoma cell line, were i.v. injected to construct the tumour model at day −14 (FIGS. 38A-38E). The Raji cells also express luciferase and green fluorescent protein (GFP) as reporters (Raji-Luc-GFP) so that the tumour and its growth are monitored in vivo. It was found that treatment with either Blinatumomab or SiTE not only induced on-target, off-tumour toxicity in the liver, but led to strong CRS (FIGS. 34A-34C, FIGS. 40A-40M, and FIGS. 41A-41J). The levels of cytokines such as interleukin-6 (IL-6) and tumour necrosis factor-α (TNF-α) were similar in Blinatumomab- and SiTE-treated mice (FIGS. 38A-38E). Tocilizumab is a commercialized drug for CRS treatment in the clinic and has been shown to prevent CRS in a humanized mouse model by preventative administration. Thus in order to solely evaluate the effect of on-target, off-tumour toxicity, Tocilizumab was administered before either Blinatumomab, SiTE or PBS was i.v. injected as treatment (FIG. 40A). Mouse body weight, serum cytokines and liver enzymes were monitored. Tumour burden was also monitored using IVIS. As shown in FIG. 40B, both Blinatumomab and SiTE treatment induced effective tumour cell killing. However, mouse body weight loss and liver enzyme release to the blood were also observed in these groups (FIGS. 40C and FIGS. 40E-40F). When >15% body weight loss (day 7) was observed, mice in the SiTE group were i.v. injected with AMD. It was found that AMD treatment can greatly decrease the liver enzyme levels and cytokine levels in blood (FIGS. 40E-40H), indicating that on-target, off-tumour toxicity has been controlled. However, if left untreated, Blinatumomab and SiTE treatment can induce higher toxicities and finally lead to mouse death (FIG. 40D). Histology analysis also confirmed that AMD can suppress the off-tumour toxicity caused by SiTE nanoparticle (FIGS. 40I-40J).

Although AMD treatment also induced the switch off of the SiTE activity, no tumour cells were detected at day 30 (FIG. 408). In order to investigate if the tumour cell clearance was a result of tumour-specific immune response, the tumour-free mice from the SiTE+AMD group were rechallenged with 10⁶ Raji-Luc-GFP cells and tumour burden over time was monitored (FIG. 40K). It was found that Raji-Luc-GFP cells were quickly cleared from the mice, demonstrating that the SiTE+AMD treatment induced an immune memory effect toward the tumour cells. To further confirm SiTE+AMD treatment induced tumour-specific T cell response, T cells in the tumour-free mice from the SiTE+AMD group were sorted and co-cultured with Raji-Luc-GFP cells for 24 h. Tumour cell viability and IFN-γ levels in the cell culture medium were determined. The results (FIGS. 40L-40M) showed that T cells in the tumour-free mice from the SiTE+AMD group induced substantial tumour cell lysis and IFN-γ release (FIGS. 40K-40M). These results demonstrate that SiTE can not only clear the tumour cells in the NSG mice, but they also induce lower off-tumour toxicity than Blinatumomab, which eventually leads to extended mice survival.

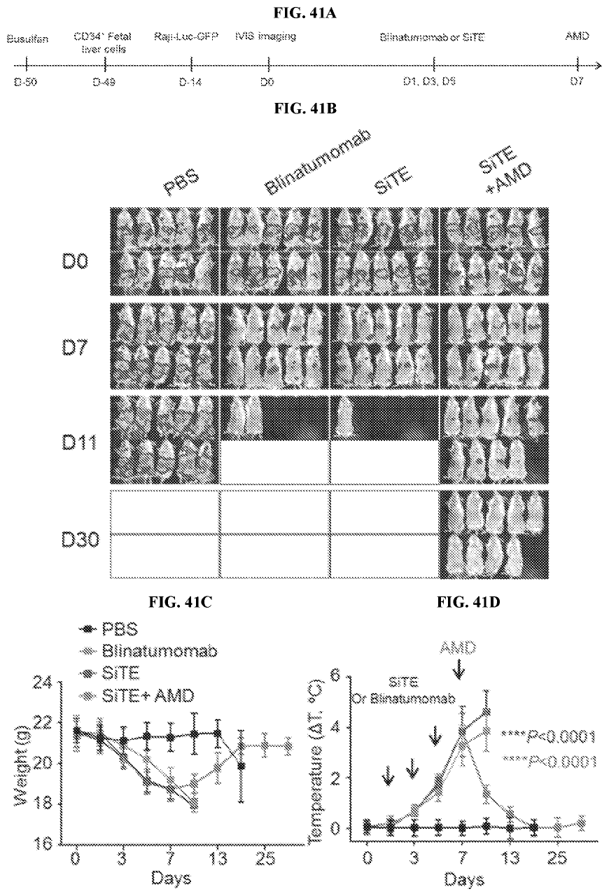

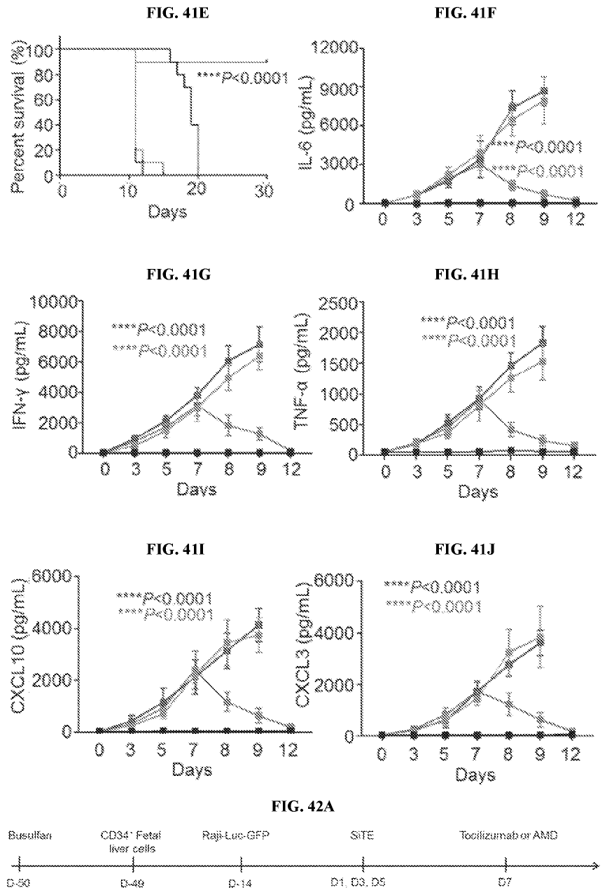

SiTE and Blinatumomab were also compared with regard to treating symptoms of CRS and neurotoxicity. The humanized mouse model was constructed as before, but this time the piggybac transposon system was not used to express CD19 in the mouse livers. This allowed better analysis of the effects of the SiTE on CRS and neurotoxicity (FIG. 41A). Raji-Luc-GFP cells were i.v. injected at day −14. Blinatumomab, SiTE or PBS were i.v. injected to the mice at day one. Mouse tumour burden, body weight, temperature, and plasma cytokine levels were monitored (FIGS. 41B-41J). As shown in FIGS. 41B-41E, both the SiTE and Blinatumomab treatments induced high fever and body weight loss shortly after treatment. Further, plasma cytokine levels revealed that IL-6, IFN-γ, TNF-α, CXCL10, and CCL3 concentrations greatly increased after both the SiTE and Blinatumomab treatments (FIGS. 41F-41J). At the first sign of toxicity (high fever, ΔT>2° C.) in the SiTE-treated mice, 10 mg/kg AMD was i.v. injected into mice. Surprisingly, the body weight loss and high fever was contained in the AMD-treated group 24 h after the treatment (FIGS. 41C-41D). Moreover, the increased cytokine levels gradually recovered to within normal ranges after 24 h post-AMD treatment (FIGS. 41F-41J). However, mice treated with Blinatumomab or SiTE alone presented with higher fever and increased weight loss at day 9, which eventually led to mouse death (FIG. 41E). Although AMD treatment also induced the switch off the antitumour activity of SiTE, tumour cells were not detected at day 30 (FIG. 41B). This is because of the eliciting of tumour-specific immune response by the high dose SiTE treatment. These results demonstrate that the SiTE strategy can help to suppress CRS and prolong survival in a humanized immune system mouse model bearing the human CD19 B cell lymphoma.

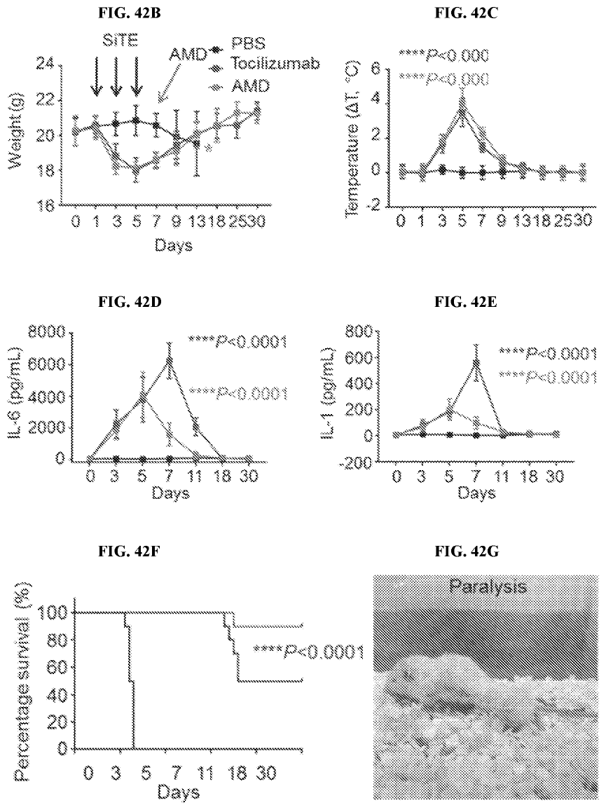

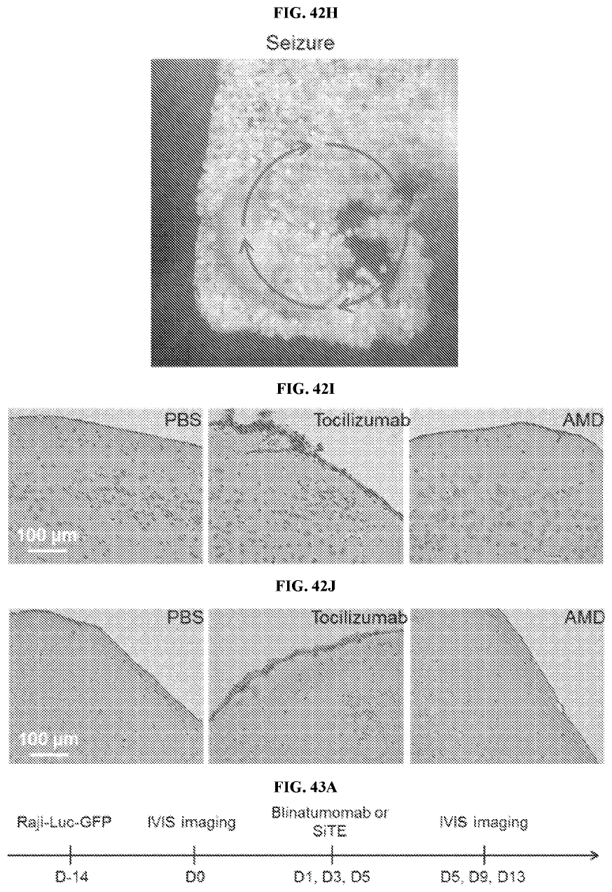

It was also evaluated if the SiTE can help control neurotoxicity. In the clinic, neurotoxicity is usually delayed and occurs several weeks after CRS onset. Tumour-bearing mice were treated with SiTE at days 1, 3 and 5 (FIG. 42A). Tocilizumab or AMD were used when signs of CRS presented (high fever, ΔT>2° C.). In this way, the symptoms of CRS can be greatly inhibited and mouse survival can be prolonged, in order to evaluate the effect of the SiTE on neurotoxicity. Rather than use a preventative strategy for treating CRS (FIGS. 40A-40M), here Tocilizumab was used to treat CRS when symptoms presented, so that it can be compared directly with the SiTE strategy. As shown in FIG. 42B, tumour-bearing NSG-SGM3 mice displayed increased body weight loss after SiTE treatment. One group was treated with Tocilizumab and the other group was treated with AMD. Mouse body weight and temperature gradually recovered to normal ranges after both the Tocilizumab and AMD treatments. AMD treatment decreased both levels of IL-6 and IL-1 cytokines in mouse blood, while Tocilizumab did not (FIGS. 42D-42E). This is consistent with previous studies since Tocilizumab is an antibody for the IL-6 receptor so it will not decrease the IL-6 concentration in blood. At around day 33, mice that received Tocilizumab developed sudden paralysis (FIG. 42G) or seizure (FIG. 42H), which are signs of lethal neurological syndrome. However, most mice in the AMD group did not develop these symptoms. Studies have shown that IL-1 is the major cause of neurotoxicity. It is hypothesized herein that the AMD-induced protection from neurotoxicity is due to the rapid recovery of IL-1 levels back to normal physiological ranges. In mice that developed paralysis and seizure in the Tocilizumab treatment group, mice did show brain meningeal thickening (FIG. 42I) accompanied by human monocyte infiltration in the subarachnoid space, as determined by immunohistochemistry analysis of human CD68 (FIG. 42J). Brain meningeal thickening and human monocyte infiltration were not observed in mice treated with AMD. Moreover, prolonged animal survival (FIG. 42F) in the AMD treatment group was observed. These results demonstrate that the SITE strategy can also protect mice from lethal neurotoxicity.

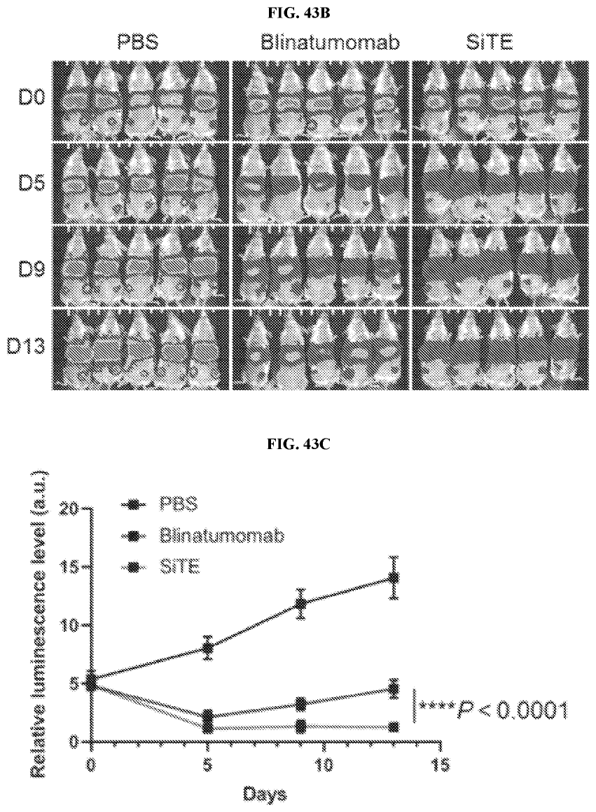

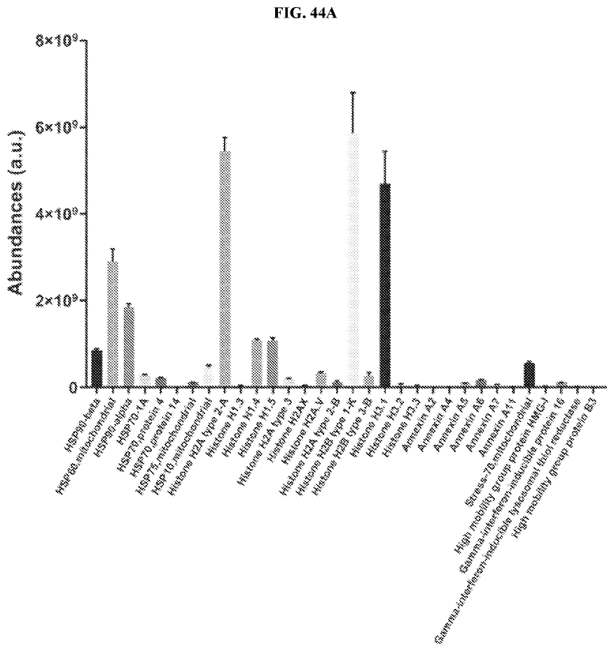

After demonstrated that SiTE is advantageous over Blinatumomab in terms of lower on-target, off-tumour toxicity, lower CRS and neurotoxicity, the antitumour efficacy of SiTE and Blinatumomab were also compared in vivo. In order to specifically assess the antitumour efficacy of Blinatumomab and the SiTE, expression of the CD19 antigen in the mouse liver was refrained from. By employing this approach, it was ensured that treatment with either Blinatumomab or SiTE would not elicit on-target, off-tumour toxicity. To further avoid the effect of CRS on the evaluation of the therapeutic outcome of Blinatumomab and SiTE, normal NSG mice were employed, devoid of humanization. These studies demonstrated that both Blinatumomab and SiTE induced substantial suppression of tumour growth (FIGS. 43B-43C). Remarkably, at day 13, the tumour burden exhibited a notably lower level in mice treated with SiTE in comparison to those treated with Blinatumomab (FIGS. 43B-43C), thereby emphasize the good antitumour efficacy of SiTE.

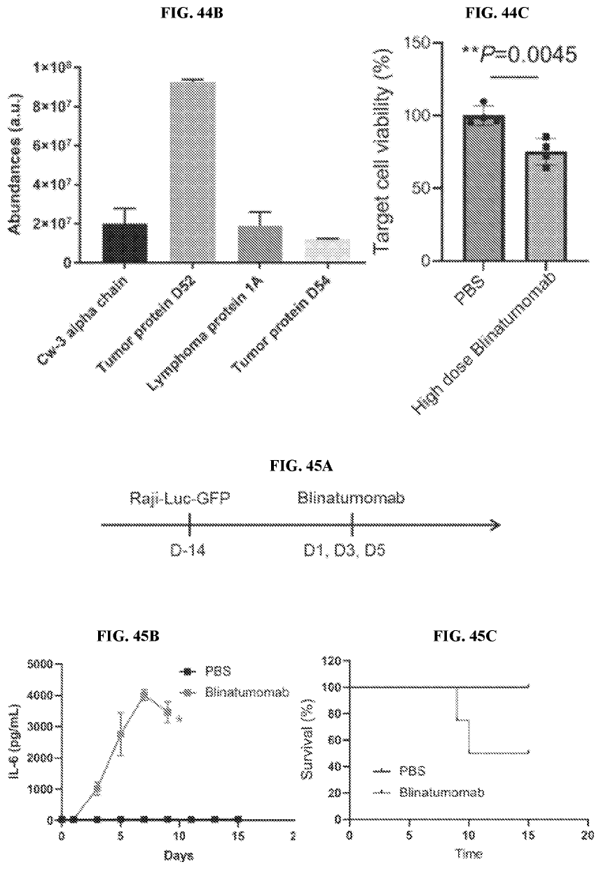

It was also investigated if Blinatumomab could also induce a vaccine effect. It was found that high dose of Blinatumomab induced substantial release of DAMPs and tumour antigens from tumour cells (FIGS. 44A-44B). Since high levels of DAMP and tumour antigen release have been demonstrated to improve antigen processing and presentation, it is highly possible that high dose of Blinatumomab can also induce an in situ vaccine effect. In order to demonstrate the vaccine effect in vivo, T cells were collected from mice treated with high dose of Blinatumomab and cultured them with Raji-Luc-GFP tumour cells. It was found that the T cells induced substantial tumour cell killing compared to T cells collected from PBS-treated mice (FIG. 44C). These results demonstrate that high dose of Blinatumomab can also induce a vaccine effect. It was also questioned whether CRS could be halted by simply discontinuing the administration of Blinatumomab, owing to its inherent short half-life. The results (FIGS. 45A-45C) revealed that mice treated with Blinatumomab exhibited the development of CRS, characterized by a notable elevation of IL-6 levels within the serum (FIG. 45B). Significantly, this surge in IL-6 persisted and ultimately led to the mortality of the mice, even after cessation of Blinatumomab treatment (FIG. 45C). This can be explained that, even though Blinatumomab shows short serum half-life, the accumulation of within tumour tissues and normal tissues expressing CD19 can persist for longer time. The pronounced targeting of T cells and CD19-expressing cells, coupled with heightened activation of many immune cells, served to amplify the cytokine cascade, rendering it impervious to interruption by the mere discontinuation of Blinatumomab usage. These findings underscore the critical exigency for the development of an alternative to Blinatumomab, one that can be switched off as needed, in this rapidly evolving research field.

### ENUMERATED EMBODIMENTS

The following exemplary embodiments are provided, the numbering of which is not to be construed as designating levels of importance:

Embodiment 1 provides a disruptable linker comprising a compound of formula (I):

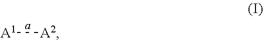

wherein:
A¹ is

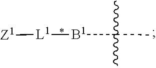

A² is

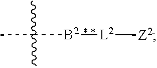

Z¹ and Z² each independently comprise a cross-linking moiety;
L¹ and L² each independently comprise a linker;
B¹ is selected from the group consisting of β-cyclodextrin (β-CD) and cucurbit[8]uril;
B² comprises a β-CD or curcurbit[8]uril binding moiety;
indicates the bond between L¹ and B¹;
* indicates the bond between L² and B²; and
bond a is an optional non-covalent bonding interaction.

Embodiment 2 provides the disruptable linker of Embodiment 1, wherein the compound of formula (I) is a compound of formula (Ia):

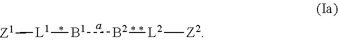

Embodiment 3 provides the disruptable linker of Embodiment 1 or 2, wherein Z¹ and Z² are each independently selected from the group consisting of

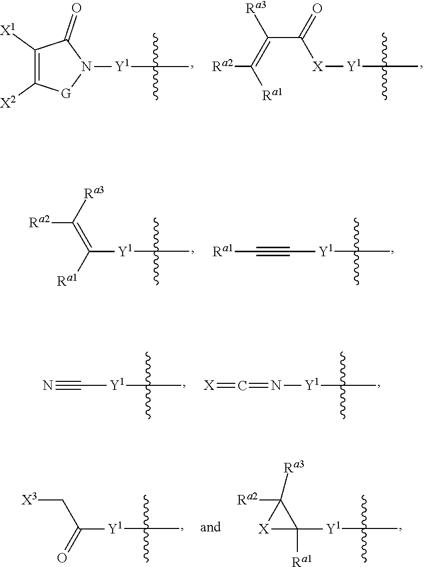

wherein:
X is selected from the group consisting of O, S, and N(R^{A});
X¹ and X² are each independently selected from the group consisting of H and halogen;
G is optionally substituted C₁-C₂ alkylene or —C(═O)—;
X³ is selected from the group consisting of I, Br, C₁, and OR^{D}.
Y¹ is selected from the group consisting of a bond, —C(═O)—, —C(═O)(C₁-C₆ alkylene)-, —C(═O)(C₁-C₆ heteroalkylene)-, —C(═O)(C₁-C₆ alkylene)C(═O)—, and —C(═O)(C₁-C₆ heteroalkylene)C(═O)—;
each occurrence of R^{a1}, R^{a2}, and R^{a3}, if present, is independently selected from the group consisting of H, halogen, CN, NO₂, optionally substituted C₁-C₆ alkyl, optionally substituted C₃-C₈ cycloalkyl, optionally substituted C₁-C₆ alkoxy, optionally substituted C₃-C₈ cycloalkoxy, heterocyclyl, phenyl, naphthyl, heteroaryl, OR^{B}, N(R^{B})(R^{C}), NO₂, C(═O)N(R^{B})(R^{C}), C(═O)R^{B}, —C(═O)OR^{B}, OC(═O)R^{B}, OC(═O)OR^{B}, SR^{B}, S(═O)R^{B}, S(═O)₂R^{B}, N(R^{B})S(═O)₂R^{C}, N(R^{B})C(═O)R^{C}, and S(═O)₂N(R^{B})(R^{C}),
each occurrence of R^{A}, R^{B}, and R^{C} is independently selected from the group consisting of H, —C(═O)(C₁-C₆ alkyl), —C(═O)(C₁-C₆ haloalkyl), optionally substituted C₁-C₆ alkyl, optionally substituted C₁-C₃ haloalkyl, optionally substituted C₃-C₈ cycloalkyl, optionally substituted C₁-C₆ alkoxy, and optionally substituted C₃-C₈ cycloalkoxy, optionally substituted phenyl, optionally substituted naphthyl, and optionally substituted heteroaryl; and
R^{D} is selected from the group consisting of H, —C(═O)R^{B}, and —S(═O)₂R^{B}.

Embodiment 4 provides the disruptable linker of Embodiment 3, wherein at least one of Z¹ and Z² is

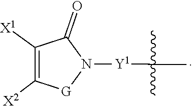

Embodiment 5 provides the disruptable linker of Embodiment 3 or 4, wherein Y¹ is —C(═O)(CH₂CH₂)—.

Embodiment 6 provides the disruptable linker of any one of Embodiments 3-5, wherein G is —C(═O)—.

Embodiment 7 provides the disruptable linker of any one of Embodiments 3-6, wherein at least one of X¹ and X² is H.

Embodiment 8 provides the disruptable linker of any one of Embodiments 1-7, wherein at least one of Z¹ and Z² is 0

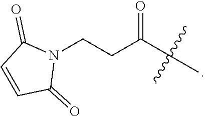 [SMILES: *C(=O)CCN1C(=O)C=CC1=O]

Embodiment 9 provides the disruptable linker of any one of Embodiments 1-8, wherein L¹ and L² are independently selected from the group consisting of a bond, -(optionally substituted C₁-C₁₂ alkylene)₁₋₁₀₀-, and -(optionally substituted C₁-C₁₂ heteroalkylene)₁₋₁₀₀-.

Embodiment 10 provides the disruptable linker of any one of Embodiments 1-9, wherein each of L¹ and L² are independently —NH(CH₂CH₂)(OCH₂CH₂)ₙNH—, wherein n is an integer between 1 and 50.

Embodiment 11 provides the disruptable linker of Embodiment 10, wherein at least one of the following:
(a) L¹ is *—NH(CH₂CH₂)(OCH₂CH₂)ₙNH—; and
(b) L² is **—NH(CH₂CH₂)(OCH₂CH₂)ₙNH—.

Embodiment 12 provides the disruptable linker of any one of Embodiments 1-11, wherein the bond indicated as * comprises at least one of the following:
(a) a covalent bond between L¹ and an exocyclic methylene of the β-CD in B¹, optionally wherein the bond is formed by displacement of a tosylated primary hydroxyl of a glucose monomer of the β-CD in B¹; and
(b) a C—N bond.

Embodiment 13 provides the disruptable linker of Embodiment 12, wherein the exocyclic methylene is a secondary hydroxyl of a glucose monomer of the β-CD in B¹.

Embodiment 14 provides the disruptable linker of Embodiment 13, wherein the bond indicated as * is formed by displacement of a tosylate by an amine in L¹.

Embodiment 15 provides the disruptable linker of any one of Embodiments 1-14, wherein B² is

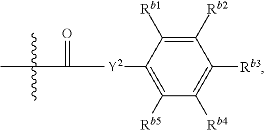

wherein:
Y² is selected from the group consisting of a bond, C₁-C₆ alkylene, and C₁-C₆ heteroalkylene; and
each occurrence of R^{b1}, R^{b2}, R^{b3}, R^{b4}, and R^{b5} if present, is independently selected from the group consisting of H, optionally substituted C₁-C₆ alkyl, optionally substituted C₃-C₈ cycloalkyl, optionally substituted C₁-C₆ alkoxy, optionally substituted C₃-C₈ cycloalkoxy, optionally substituted C₂-C₈ heterocycloalkyl, optionally substituted phenyl, optionally substituted naphthyl, optionally lithocholic acid, optionally coumarin, optionally ferrocene, and optionally substituted heteroaryl.

Embodiment 16 provides the disruptable linker of Embodiment 15, wherein Y₂ is —CH₂CH₂—.

Embodiment 17 provides the disruptable linker of any one of Embodiments 15-16, wherein at least one of the following:
(a) at least one of R^{b1}, R^{b2}, R^{b3}, R^{b4}, and R^{b5} is H;
(b) at least two of R^{b1}, R^{b2}, R^{b3}, R^{b4}, and R^{b5} are H;
(c) at least three of R^{b1}, R^{b2}, R^{b3}, R^{b4}, and R^{b5} are H;
(d) at least four of R^{b1}, R^{b2}, R^{b3}, R^{b4}, and R^{b5} are H; and
(e) each of R^{b1}, R^{b2}, R^{b3}, R^{b4}, and R^{b5} are H.

Embodiment 18 provides the disruptable linker of any one of Embodiments 15-17, wherein B² is

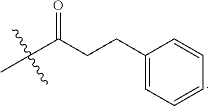 [SMILES: *C(=O)CCc1ccccc1]

Embodiment 19 provides the disruptable linker of any one of Embodiments 1-18, wherein A¹ is

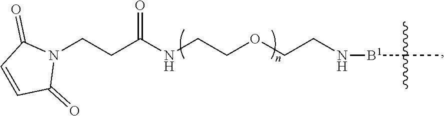

wherein n is selected from the group consisting of 35, 36, 37, 38, 39, 40, 41, 42, 43, 44, and 45.

Embodiment 20 provides the disruptable linker of any one of Embodiments 1-19, wherein A² is

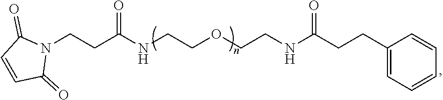

wherein n is selected from the group consisting of 35, 36, 37, 38, 39, 40, 41, 42, 43, 44, and 45.

Embodiment 21 provides the disruptable linker of any one of Embodiments 1-20, wherein the non-covalent bonding interaction of bond a is disrupted with a disrupting agent.

Embodiment 22 provides the disruptable linker of Embodiment 21, wherein the disrupting agent is selected from the group consisting of amantadine and an amino acid, optionally wherein the amino acid is an amino acid comprising an aromatic moiety, optionally wherein the amino acid comprising an aromatic moiety is phenylalanine or tyrosine.

Embodiment 23 provides a switchable Bispecific T cell Engager (switch-BiTE) comprising:
(a) a first binding domain specific for a surface antigen on a target cell;
(b) a second binding domain specific for a surface antigen on an immune effector cell; and
(c) the disruptable linker of any one of Embodiments 1-22.

Embodiment 24 provides the switch-BiTe of Embodiment 23, wherein bond a is present.

Embodiment 25 provides the switch-BiTe of Embodiment 23 or 24, wherein either of the following:
(i) the cross-linking moiety in Z¹ is covalently bonded to the first binding domain and the cross-linking moiety in Z² is covalently bonded to the second binding domain: or
(ii) the cross-linking moiety in Z¹ is covalently bonded to the second binding domain and the cross-linking moiety in Z² is covalently bonded to the first binding domain.

Embodiment 26 provides the switch-BiTE of any one of 23-25, wherein the first binding domain is selected from the group consisting of a Fab, a single-chain variable fragment (scFv), a single-domain antibody, a full-length antibody and a receptor ligand.

Embodiment 27 provides the switch-BiTE of any one of Embodiments 23-26, wherein the first binding domain is a Fab.

Embodiment 28 provides the switch-BiTE of any one of Embodiments 23-27, wherein the surface antigen on the target cell is a tumor-associated antigen.

Embodiment 29 provides the switch-BiTE of Embodiment 28, wherein the tumor-associated antigen is selected from the group consisting of BCMA, C-Met, CD19, CD20, CEA, EGFR, EphA2, HER2, MART1, Mesothelin, MUC1, NY-ESO-1, PD-L1, PSCA, PSMA, ROR1, VEGFR2

Embodiment 30 provides the switch-BiTE of any one of Embodiments 23-29, wherein the surface antigen on the target cell is HER2.

Embodiment 31 provides the switch-BiTE of any one of Embodiments 23-30, wherein the target cell is a tumor cell.

Embodiment 32 provides the switch-BiTE of any one of Embodiments 23-31, wherein the second binding domain is selected from the group consisting of consisting of a Fab, a single-chain variable fragment (scFv), a full-length antibody, and a single-domain antibody.

Embodiment 33 provides the switch-BiTE of Embodiment 32, wherein the second binding domain is a Fab.

Embodiment 34 provides the switch-BiTE of any one of Embodiments 23-33, wherein the immune effector cell is a T cell.

Embodiment 35 provides the switch-BiTE of any one of Embodiments 23-34, wherein the second domain is specific for CD3.

Embodiment 36 provides the switch-BiTE of any one of Embodiments 23-35, wherein the second domain is specific for CD3ε.

Embodiment 37 provides the switch-BiTE of any one of Embodiments 23-36, wherein the disruptable linker comprises Phenyl-PEG-MAL and β-CD-PEG-MAL.

Embodiment 38 provides the switch-BiTE of any one of Embodiments 23-37, wherein bond a of the disruptable linker is disrupted with a disrupting agent.

Embodiment 39 provides the switch-BiTE of Embodiment 37 or 38, wherein the disrupting agent has a higher affinity for β-CD than does Phenyl-PEG-MAL.

Embodiment 40 provides the switch-BiTE of Embodiment 38 or 39, wherein the disrupting agent is selected from the group consisting of amantadine and an amino acid, optionally wherein the amino acid is an amino acid comprising an aromatic moiety, optionally wherein the amino acid comprising an aromatic moiety is phenylalanine or tyrosine.

Embodiment 41 provides a method for generating the switchable bispecific T cell engager (switch-BiTE) of Embodiment 23, comprising:
(a) conjugating the first and second binding domains with A¹ and A² of the disruptable linker to provide a first engaging fragment and a second engaging fragment, under either of the following conditions:
   (i) A¹ is conjugated to the first binding domain and A² is conjugated to the second binding domain; or
   (ii) A¹ is conjugated to the second binding domain and A² is conjugated to the first binding domain; and
(b) contacting the first engaging fragment and the second engaging fragment.

Embodiment 42 provides the method of Embodiment 41, wherein in step (a) bond a in the disruptable linker is absent.

Embodiment 43 provides the method of Embodiment 41 or 42, wherein the contacting in step (b) generates a non-covalent bonding interaction between the first engaging fragment and the second engaging fragment.

Embodiment 44 provides the method of any one of Embodiments 41-43, wherein at least one of Z¹ and Z² comprises an α,β-unsaturated carbonyl moiety.

Embodiment 45 provides the method of any one of Embodiments 41-44, wherein at least one of Z¹ and Z² is

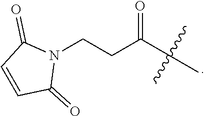 [SMILES: *C(=O)CCN1C(=O)C=CC1=O]

Embodiment 46 provides the method of Embodiment 44 or 45, wherein at least one of the first binding domain and the second binding domain is treated with 2-iminothiolane.

Embodiment 47 provides the method of Embodiment 46, wherein at least one of Z¹ or Z² is conjugated to one of the first binding domain or the second binding domain via a Michael addition (i.e., 1,4-conjugate addition).

Embodiment 48 provides the method of any one of Embodiments 42-47, wherein:
(a) A¹ comprises

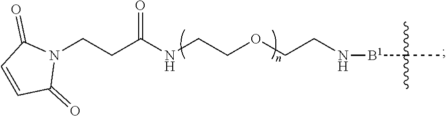

(b) A² comprises

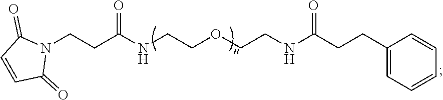

and
(c) each occurrence of n is independently selected from the group consisting of 35, 36, 37, 38, 39, 40, 41, 42, 43, 44 and 45.

Embodiment 49 provides the method of any one of Embodiments 41-48, wherein the first binding domain and second binding domain comprise Fab fragments from two or more antibodies.

Embodiment 50 provides the method of Embodiment 49, wherein one or more of the Fab fragments is specific for a surface antigen on an immune effector cell.

Embodiment 51 provides the method of Embodiment 50, wherein the immune effector cell is a T cell.

Embodiment 52 provides the method of Embodiment 50 or 51, wherein the surface antigen is CD3.

Embodiment 53 provides the method of any one of Embodiments 50-52, wherein the surface antigen is CD3ε.

Embodiment 54 provides the method of Embodiment 50, wherein one or more of the antibody Fab fragments is specific for a surface antigen on a target cell.

Embodiment 55 provides the method of Embodiment 54, wherein the surface antigen is a tumor-associated antigen.

Embodiment 56 provides the method of Embodiment 55, wherein the tumor-associated antigen is selected from the group consisting of BCMA, C-Met, CD19, CD20, CEA, EGFR, EphA2, HER2, MART1, Mesothelin, MUC1, NY-ESO-1, PD-L1, PSCA, PSMA, ROR1, VEGFR2

Embodiment 57 provides the method of Embodiment 56, wherein the tumor-associated antigen is HER2.

Embodiment 58 provides the method of Embodiment 54, wherein the target cell is a tumor cell.

Embodiment 59 provides a method of treating, ameliorating, and/or preventing cancer in a subject in need thereof, comprising administering to the subject an effective amount of the switch-BiTE of any one of Embodiments 23-40, thereby treating the cancer.

Embodiment 60 provides the method of Embodiment 59, further comprising administering to the subject an effective amount of a disrupting agent.

Embodiment 61 provides the method of Embodiment 60, wherein administration of the disrupting agent treats, prevents, and/or ameliorates at least one off-tumor effect caused by the switch-BiTE.

Embodiment 62 provides the method of Embodiment 60 or 61, wherein the disrupting agent is selected from the group consisting of amantadine and an amino acid, optionally wherein the amino acid is an amino acid comprising an aromatic moiety, optionally wherein the amino acid comprising an aromatic moiety is phenylalanine or tyrosine.

Embodiment 63 provides the method of any one of Embodiments 59-62, wherein the cancer is a hematologic cancer.

Embodiment 64 provides the method of Embodiment 63, wherein the hematologic cancer is selected from the group consisting of a leukemia, a lymphoma, and a myeloma.

Embodiment 65 provides the method of any one of Embodiments 59-62, wherein the cancer is a solid cancer.

Embodiment 66 provides a method for generating an immune response against a target cell in a subject in need thereof, comprising:
(a) administering to the subject an amount of the switch-BiTE of any one of Embodiments 23-40 sufficient to prime an immune response; and
(b) administering to the subject an effective amount of a small molecule capable of disrupting the switch-BiTE of step (a).

Embodiment 67 provides the method of Embodiment 66, wherein the immune response is against target cell antigens other than those targeted by the switch-BiTE.

Embodiment 68 provides the method of Embodiment 66, wherein the immune response continues after the disruption of the switch-BiTE.

Embodiment 69 provides the method of Embodiment 66, wherein the target cell is a cancer cell.

Embodiment 70 provides a method for generating an anti-tumor immune response in a subject in need thereof, comprising:
(a) administering to the subject an amount of the switch-BiTE of any one of Embodiments 23-40 sufficient to prime an anti-tumor immune response; and
(b) administering to the subject an effective amount of a small molecule capable of disrupting the switch-BiTE of step (a).

Embodiment 71 provides the method of Embodiment 70, wherein the anti-tumor immune response is against antigens other than those targeted by the switch-BiTE.

Embodiment 72 provides the method of Embodiment 70, wherein the anti-tumor immune response continues after the disruption of the switch-BiTE.

The terms and expressions employed herein are used as terms of description and not of limitation, and there is no intention in the use of such terms and expressions of excluding any equivalents of the features shown and described or portions thereof, but it is recognized that various modifications are possible within the scope of the embodiments of the present application. Thus, it should be understood that although the present application describes specific embodiments and optional features, modification and variation of the compositions, methods, and concepts herein disclosed may be resorted to by those of ordinary skill in the art, and that such modifications and variations are considered to be within the scope of embodiments of the present application.

1. A disruptable linker comprising a compound of formula (I): 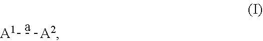
wherein:
A¹ is 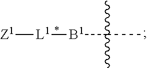
A² is 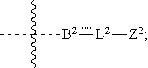
Z¹ and Z² each independently comprise a cross-linking moiety;
L¹ and L² each independently comprise a linker;
B¹ is selected from the group consisting of β-cyclodextrin (β-CD) and cucurbit[8]uril;
B² comprises a β-CD or curcurbit[8]uril binding moiety;
* indicates the bond between L¹ and B¹;
** indicates the bond between L² and B²; and
bond a is an optional non-covalent bonding interaction.

2. The disruptable linker of claim 1, wherein Z¹ and Z² are each independently selected from the group consisting of 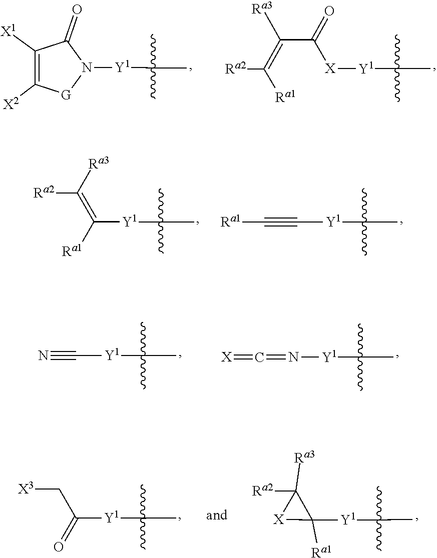
wherein:
X is selected from the group consisting of O, S, and N(R^{A});
X¹ and X² are each independently selected from the group consisting of H and halogen;
G is optionally substituted C₁-C₂ alkylene or —C(═O)—;
X³ is selected from the group consisting of I, Br, Cl, and OR^{D};
Y¹ is selected from the group consisting of a bond, —C(═O)—, —C(═O)(C₁-C₆ alkylene)-, —C(═O)(C₁-C₆ heteroalkylene)-, —C(═O)(C₁-C₆ alkylene)C(═O)—, and —C(═O)(C₁-C₆ heteroalkylene)C(═O)—;
each occurrence of R^{a1}, R^{a2}, and R^{a3}, if present, is independently selected from the group consisting of H, halogen, CN, NO₂, optionally substituted C₁-C₆ alkyl, optionally substituted C₃-C₈ cycloalkyl, optionally substituted C₁-C₆ alkoxy, optionally substituted C₃-C₈ cycloalkoxy, heterocyclyl, phenyl, naphthyl, heteroaryl, OR^{B}, N(R^{B})(R^{C}), NO₂, C(═O)N(R^{B})(R^{C}), C(═O)R^{B}, —C(═O)OR^{B}, OC(═O)R^{B}, OC(═O)OR^{B}, SR^{B}, S(═O)R^{B}, S(═O)₂R^{B}, N(R^{B})S(═O)₂R^{C}, N(R^{B})C(═O)R^{C}, and S(═O)₂N(R^{B})(R^{C});
each occurrence of R^{A}, R^{B}, and R^{C} is independently selected from the group consisting of H, —C(═O)(C₁-C₆ alkyl), —C(═O)(C₁-C₆ haloalkyl), optionally substituted C₁-C₆ alkyl, optionally substituted C₁-C₃ haloalkyl, optionally substituted C₃-C₈ cycloalkyl, optionally substituted C₁-C₆ alkoxy, and optionally substituted C₃-C₈ cycloalkoxy, optionally substituted phenyl, optionally substituted naphthyl, and optionally substituted heteroaryl; and
R^{D} is selected from the group consisting of H, —C(═O)R^{B}, and —S(═O)₂R^{B}.

3. The disruptable linker of claim 2, wherein at least one of the following applies:
(a) at least one of Z¹ and Z² is 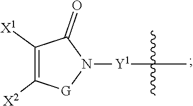
(b) Y¹ is —C(═O)(CH₂CH₂)—;
(c) G is —C(═O)—;
(d) at least one of X¹ and X² is H.

4-7. (canceled)

8. The disruptable linker of claim 1, wherein L¹ and L² are independently selected from the group consisting of a bond, -(optionally substituted C₁-C₁₂ alkylene)₁₋₁₀₀-, and -(optionally substituted C₁-C₁₂ heteroalkylene)₁₋₁₀₀-, optionally wherein each of L¹ and L² are independently —NH(CH₂CH₂)(OCH₂CH₂)ₙNH—, wherein n is an integer between 1 and 50, optionally wherein L¹ is *—NH(CH₂CH₂)(OCH₂CH₂)ₙNH, and optionally wherein L² is **—NH(CH₂CH₂)(OCH₂CH₂)ₙNH—.

9-10. (canceled)

11. The disruptable linker of claim 1, wherein
the bond indicated as * comprises at least one of the following:
(a) a covalent bond between L¹ and an exocyclic methylene of the β-CD in B¹, optionally wherein the bond is formed by displacement of a tosylated primary hydroxyl of a glucose monomer of the β-CD in B¹; and
(b) a C—N bond.

12-13. (canceled)

14. The disruptable linker of claim 1, wherein B2 is 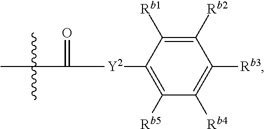
wherein:
Y² is selected from the group consisting of a bond, C₁-C₆ alkylene, and C₁-C₆ heteroalkylene; and
each occurrence of R^{b}, R^{b2}, R^{b3}, R^{b4}, and R^{b5} if present, is independently selected from the group consisting of H, optionally substituted C₁-C₆ alkyl, optionally substituted C₃-C₈ cycloalkyl, optionally substituted C₁-C₆ alkoxy, optionally substituted C₃-C₈ cycloalkoxy, optionally substituted C₂-C₈ heterocycloalkyl, optionally substituted phenyl, optionally substituted naphthyl, optionally lithocholic acid, optionally coumarin, optionally ferrocene, and optionally substituted heteroaryl.

15. (canceled)

16. The disruptable linker of claim 14, wherein Y² is —CH₂CH₂— and/or at least one of the following applies:
(a) at least one of R^{b1}, R^{b2}, R^{b3}, R^{b4}, and R^{b5} is H;
(b) at least two of R^{b1}, R^{b2}, R^{b3}, R^{b4}, and R^{b5} are H;
(c) at least three of R^{b}, R^{b2}, R^{b3}, R^{b4}, and R^{b5} are H;
(d) at least four of R^{b1}, R^{b2}, R^{b3}, R^{b4}, and R^{b5} are H; and
(e) each of R^{b1}, R^{b2}, R^{b3}, R^{b4}, and R^{b5} are H.

17. (canceled)

18. The disruptable linker of claim 1, wherein at least one of the following applies:
(a) A¹ is 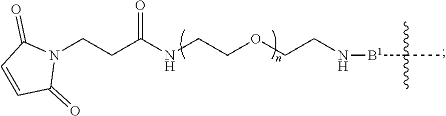
(b) A² is 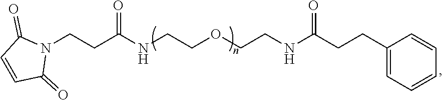
wherein each n is independently selected from the group consisting of 35, 36, 37, 38, 39, 40, 41, 42, 43, 44, and 45.

19. (canceled)

20. The disruptable linker of claim 1, wherein the non-covalent bonding interaction of bond a is disrupted with a disrupting agent, optionally wherein the disrupting agent is selected from the group consisting of amantadine and an amino acid, optionally wherein the amino acid is an amino acid comprising an aromatic moiety, optionally wherein the amino acid comprising an aromatic moiety is phenylalanine or tyrosine.

21. (canceled)

22. A switchable Bispecific T cell Engager (switch-BiTE) comprising:
(a) a first binding domain specific for a surface antigen on a target cell;
(b) a second binding domain specific for a surface antigen on an immune effector cell; and
(c) the disruptable linker of claim 1.

23. (canceled)

24. The switch-BiTe of claim 22, wherein either of the following:
(i) the cross-linking moiety in Z¹ is covalently bonded to the first binding domain and the cross-linking moiety in Z² is covalently bonded to the second binding domain; or
(ii) the cross-linking moiety in Z¹ is covalently bonded to the second binding domain and the cross-linking moiety in Z² is covalently bonded to the first binding domain.

25. The switch-BiTE of claim 22, wherein at least one of the following applies:
(a) the first binding domain is selected from the group consisting of a Fab, a single-chain variable fragment (scFv), a single-domain antibody, a full-length antibody and a receptor ligand,
(b) the surface antigen on the target cell is a tumor-associated antigen;
(c) the target cell is a tumor cell;
(d) the second binding domain is selected from the group consisting of consisting of a Fab, a single-chain variable fragment (scFv), a full-length antibody, and a single-domain antibody;
(e) the immune effector cell is a T cell;
(f) the second domain is specific for CD3;
(g) the disruptable linker comprises Phenyl-PEG-MAL and β-CD-PEG-MAL; and
(h) the disrupting agent is selected from the group consisting of amantadine and an amino acid, optionally wherein the amino acid is an amino acid comprising an aromatic moiety, optionally wherein the amino acid comprising an aromatic moiety is phenylalanine or tyrosine.

26-39. (canceled)

40. A method for generating the switchable bispecific T cell engager (switch-BiTE) of claim 22, wherein bond a is present, the method comprising:
(a) conjugating the first and second binding domains with A¹ and A² of the disruptable linker to provide a first engaging fragment and a second engaging fragment, under either of the following conditions:
(i) A¹ is conjugated to the first binding domain and A² is conjugated to the second binding domain; or
(ii) A¹ is conjugated to the second binding domain and A² is conjugated to the first binding domain; and
(b) contacting the first engaging fragment and the second engaging fragment.

41-46. (canceled)

47. The method of claim 41, wherein:
(a) A¹ comprises 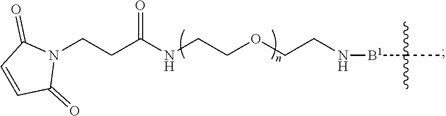
(b) A² comprises 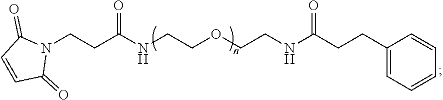
and
(c) each occurrence of n is independently selected from the group consisting of 35, 36, 37, 38, 39, 40, 41, 42, 43, 44 and 45.

48-57. (canceled)

58. A method of treating, ameliorating, or preventing cancer in a subject in need thereof, comprising administering to the subject an effective amount of the switch-BiTE of claim 22, thereby treating the cancer.

59. The method of claim 58, further comprising administering to the subject an effective amount of a disrupting agent.

60-64. (canceled)

65. A method for generating an immune response against a target cell in a subject in need thereof, comprising:
(a) administering to the subject an amount of the switch-BiTE of claim 22 sufficient to prime an immune response; and
(b) administering to the subject an effective amount of a small molecule capable of disrupting the switch-BiTE of step (a).

66. The method of claim 65, wherein at least one of the following applies:
(a) the immune response is against target cell antigens other than those targeted by the switch-BiTE;
(b) the immune response continues after the disruption of the switch-BiTE; and
(c) the target cell is a cancer cell.

67-68. (canceled)

69. A method for generating an anti-tumor immune response in a subject in need thereof, comprising:
(a) administering to the subject an amount of the switch-BiTE of claim 22 sufficient to prime an anti-tumor immune response; and
(b) administering to the subject an effective amount of a small molecule capable of disrupting the switch-BiTE of step (a).

70. The method of claim 69, wherein at least one of the following applies:
(a) the anti-tumor immune response is against antigens other than those targeted by the switch-BiTE; and
(b) the anti-tumor immune response continues after the disruption of the switch-BiTE.

71. (canceled)
